(19) United States
(12) Patent Application Publication
(10) Pub. No.: US 2026/0250340 A1
(43) Pub. Date: Aug. 27, 2026

(54) SFGF1-FGF21: A CHIMERIC PROTEIN WITH INCREASED STABILITY AND BIOLOGICAL ACTIVITIES

(71) Applicant: THE BOARD OF TRUSTEES OF THE UNIVERSITY OF ARKANSAS, Little Rock, AR (US)

(72) Inventors: Phuc PHAN, Fayetteville, AR (US); Deborah OKYERE, Farmington, AR (US); Divya GOLLAPALLI, Fayetteville, AR (US); Jingyi CHEN, Fayetteville, AR (US); Kyle QUINN, Fayetteville, AR (US); Suresh THALLAPURANAM, Fayetteville, AR (US)

(21) Appl. No.: 19/543,674

(22) Filed: Feb. 18, 2026

Related U.S. Application Data
(60) Provisional application No. 63/759,866, filed on Feb. 18, 2025.

(51) Int. Cl. C07K 14/50; A61F 13/00; A61K 38/00

(52) U.S. Cl. CPC C07K 14/501; A61F 13/00063; A61K 38/00; C07K 2319/21

(57) ABSTRACT

The present invention provides fusion proteins comprising a fibroblast growth factor 1 (FGF1) peptide and a fibroblast growth factor 21 (FGF21) peptide. Pharmaceutical compositions and wound dressings comprising the fusion proteins, polynucleotides encoding the fusion proteins, and methods of using the fusion proteins to treat a condition in a subject are also provided.

### CROSS-REFERENCE TO RELATED APPLICATIONS

This application claims priority to U.S. Provisional Application No. 63/759,866 filed on Feb. 18, 2025, the contents of which are incorporated by reference in their entirety.

### STATEMENT REGARDING FEDERALLY SPONSORED RESEARCH

This invention was made with government support under grant numbers P20GM139768 and R15GM154267 awarded by the National Institutes of Health and grant number DE-FG02-01ER15161 awarded by the United States Department of Energy. The government has certain rights in this invention.

### SEQUENCE LISTING

A Sequence Listing accompanies this application and is submitted as an XML file of the sequence listing named “16987900175_SL.xml” which is 20,992 bytes in size and was created on Feb. 18, 2026. The sequence listing is electronically submitted via Patent Center with the application and is incorporated herein by reference in its entirety.

### BACKGROUND

In 400-500 A. D. in ancient India, diabetes, known at the time as ‘honey urine’, was classified into two types, later renamed as Type 1 and Type 2 diabetes. Type 2 diabetes mellitus (T2DM) is characterized as a disease in which patients are both resistant to insulin and deficient in insulin due to pancreatic B-cell dysfunction. Globally, there is an ongoing epidemic of diabetes due to aging populations and increases in life expectancy, obesity, and stationary lifestyles. It is estimated that the prevalence of diabetes will increase steadily in the following decades. Affected patients face an overwhelming cost of treatment and experience high socioeconomic pressures to continue treatment. Patients with diabetes spent $19,736 annually on medical expenses, of which $12,022 was specifically towards diabetes, in 2022. These expenditures were reported to be 2.6 times higher than patients without diabetes. Diabetes also costs the United States $412.9 billion in both direct and indirect medical costs. Besides the economic effects, T2DM also causes considerable damage to patients' health and well-being. T2DM patients' most common cause of death is cardiovascular disease. Diabetes is also linked to kidney failure, limb amputations, and blindness. Patients are required to carefully manage their blood pressure, blood glucose, and blood lipid concentrations to minimize the risks of fatal complications and disease progression.

Non-alcoholic fatty liver disease (NAFLD) is another disease that is associated with T2DM. Patients with NAFLD/non-alcoholic steatohepatitis (NASH) have a higher risk (about 2-3 times higher) of developing T2DM, and these diseases share similar metabolic syndrome-like symptoms, such as hypertension, abdominal fat accumulation, and fasting hyperglycemia.

In 2000, fibroblast growth factor 21 (FGF21) was identified as a circulating protein that affects both insulin sensitivity and lipid regulation. FGF21 has since shown promise as a treatment for metabolic syndromes like obesity and T2DM in animal models (i.e., rodents and monkeys). Additionally, FGF21 has been shown to increase the fatty acid oxidation rate in the liver. Therefore, FGF21 has been tested in clinical trials as a treatment for NASH, and has generated positive results. FGF21 is thought to induce these effects by binding to both an FGF receptor (via its N-terminus) and the co-receptor β-Klotho (via its C-terminus).

Unfortunately, FGF21 suffers from instability, low thermal stability, low half-life, and susceptibility to proteolysis, and is therefore not an effective commercial treatment for metabolic diseases. Many efforts to increase the stability of this protein have been made, including through mutation, conjugated analogs, and fusion proteins. However, the resulting FGF21 analogs with increased stability have suffered from either a low/inconsistent glycemic effect in humans or unwanted side-effects. Therefore, although FGF21 has great therapeutic potential for treating both T2DM and NASH, analogs with improved stability, shelf-life, and bioactivity are still needed in the art.

### SUMMARY

In a first aspect, fusion proteins comprising a fibroblast growth factor 1 (FGF1) peptide and a fibroblast growth factor 21 (FGF21) peptide are provided.

In a second aspect, pharmaceutical compositions comprising a fusion protein described herein and a pharmaceutically acceptable carrier are provided.

In a third aspect, polynucleotides encoding the fusion protein described herein are provided.

In a fourth aspect, wound dressings comprising the fusion protein or pharmaceutical composition described herein are provided.

In a fifth aspect, methods of treating a condition in a subject are provided. The methods comprise administering a fusion protein, pharmaceutical composition, or wound dressing described herein to the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Structure prediction of sFGF1-FGF21 using Colabfold (AlphaFold 2). The colors of the model correspond to confidence for each region. Blue-colored regions are low in error estimate with high confidence (pLDDT 90-100), light blue is medium-high confidence (pLDDT 70-90), yellow is medium-low confidence (pLDDT 50-70), and red is low confidence with high error estimate (pLDDT 0-50).

FIGS. 2A-2C. Purification of sFGF1-FGF21 and sFGF1. Chromatogram of sFGF1-FGF21 expressed in BL21Star (DE3) using Ni²⁺-Sepharose column (FIG. 2A). FIG. 2B shows purification of wild-type sFGF1-FGF21 after large scale expression, cell harvest, sonication, purification via nickel affinity chromatography, and SDS-PAGE. Column 1 is unbound, column 2 is 50 mM imidazole, column 3 is 100 mM imidazole, column 4 is the ladder, column 5 is 250 mM imidazole, column 6 is 500 mM imidazole, column 7 is the supernatant, and column 8 is the pellet. sFGF1-FGF21 protein can be seen in the 100 mM imidazole fraction (lane 3) between the 15 and 30 kDa protein ladder markers (lane 4). Protein was shown to elute at 100 nM IMD and confirmed through an SDS-PAGE run after purification. (FIG. 2B) The fraction was concentrated, and the buffer was exchanged to exclude other contaminants and ran on a gel to confirm purity. Lane 1 is S121 (sFGF1-FGF21) and lane 2 is the ladder. FIG. 2C, left side His-tagged sFGF1-FGF21 was confirmed via western blot using an anti-His antibody, showing that the obtained product was His-tagged sFGF1-FGF21 (FIG. 2C, right hand side).

FIGS. 3A-3D. The far-UV CD and intrinsic fluorescence spectra of wtFGF1, sFGF1, sFGF1-FGF21, and rFGF21. FIG. 3A displayed the CD spectra of wtFGF1, sFGF1, sFGF1-FGF21, and rFGF21 in molar ellipticity. wtFGF1, sFGF1, and sFGF1-FGF21 all have one negative trough around 208 nm and a positive peak around 228 nm, consistent with a β-trefoil structure. rFGF21 has one negative trough at 208 nm, indicating the protein has the β-structured core but lacks the 228 nm positive peak. (FIG. 3B) The panel showed the intrinsic fluorescence of wtFGF1, sFGF1, sFGF1-FGF21, and rFGF21, all are tyrosine emission at 308 nm, indicating correct tertiary folding of wtFGF1, sFGF1, and sFGF1-FGF21. sFGF1-FGF21 has similar intrinsic tyrosine fluorescence to FGF1 and sFGF1, with tryptophan emission being quenched due to the nearby microenvironment with nitrogen-containing residues. wtFGF1 is a black line with circles, sFGF1 is a black line with upside-down triangles, sFGF1-FGF21 is a black line with triangles and rFGF21 is a black line with squares. (FIG. 3C) sFGF1 tryptophan quenching is shown with blue color representing folded protein peaks at 308 nm, and orange is unfolded protein peaks at 350 nm. In FIG. 3D, sFGF1-FGF21 tryptophan quenching is shown with blue color representing folded protein peaks at 308 nm, and orange is unfolded protein peaks at 350 nm (FIG. 3D).

FIGS. 4A-4B. Thermal and chemical stability of wtFGF1, sFGF1, sFGF1-FGF21 and rFGF21. The unfolding process of wtFGF1, sFGF1, sFGF1-FGF21, and rFGF21 using DSC was shown in FIG. 4A. wtFGF1 showed a Tm around 45.47° C. (±0.14° C.). sFGF1 showed a Tm around 68.04° C. (±0.43° C.). sFGF1-FGF21 showed a Tm around 65.92° C. (±0.1° C.). IFGF21 showed a similar Tm around 46.7° C. (±0.1° C.) but also has a second Tm at 67.15° C. (±0.14° C.) and a broad peak. FIG. 4B showed the chemical stability of wtFGF1, sFGF1, sFGF1-FGF21, and rFGF21 in denaturant (8 M urea). Cm of FGF21 is 0.42 M (±7.35%), wtFGF1 is 1.15 M (±2.36%), sFGF1-FGF21 at 3.26 M (±4.50%) and sFGF1 at 3.63 M (±16.49%). wtFGF1 is a black line with circles, sFGF1 is a black line with upside-down triangles, sFGF1-FGF21 is a black line with triangles and rFGF21 is a black line with squares.

FIGS. 5A-5D. Isothermogram of wtFGF1, sFGF1, sFGF1-FGF21 and rFGF21. The panels showed affinities to heparin of wtFGF1 (FIG. 5A), sFGF1 (FIG. 5B), sFGF1-FGF21 (FIG. 5C), and rFGF21 (FIG. 5D). Only wtFGF1 showed binding affinity to heparin, Kd~2.88 μM (±1.32) μM. sFGF1, sFGF1-FGF21, and rFGF21 do not display any heparin binding affinity.

FIGS. 6A-6B. Stability of sFGF1-FGF21 over time. FIG. 6A shows the stability of sFGF1-FGF21 in storage over nine days at three different temperatures (from top to bottom, −20° C., 4° C., 25° C.). All samples were from the sample batch and in the same storage conditions. The chimeric protein in regular conditions degraded rapidly at 25° C., slowly at 4° C., and remained mostly intact at −20° C. storage temperature. This trend of degradation was quantified from gels using densitometric scan software and compared to each other (FIG. 6B). sFGF2-FGF21 in storage with one mM PMSF conditions remained mostly intact at all storage temperatures. This trend of degradation was quantified from gels using densitometric scan software and compared to each other. Chimeric protein degradation was run on SDS-PAGE. The subsequent western blot using anti-His antibody to confirm degradation over time was at the protein's C-terminus.

FIG. 7 shows endogenous fatty acid oxidation assay results. Oxygen consumption rate due to endogenous fatty acid oxidation of mature 3T3 L1 adipocytes after exposure to four proteins, namely FGF1, sFGF1, sFGF1-FGF21, and FGF21 (n=10). sFGF1-FGF21 showed higher activity than the other proteins and the control at a concentration of 0.625 nM.

FIG. 8 shows exogenous fatty acid oxidation assay results. Oxygen consumption rate due to exogenous fatty acid oxidation of mature 3T3 L1 adipocytes after exposure to palmitate and four proteins, namely FGF1 (F1), sFGF1 (S1), sFGF1-FGF21 (S121), and FGF21 (F21). sFGF1-FGF21 showed higher activity than the control, FGF1, and sFGF1 at a concentration of 0.625 nM. sFGF1-FGF21 does not show any difference in activity when compared to FGF21. Data is presented as mean±s. d. (n=5). For multiple comparisons, one-way ANOVA with Bonferroni test was performed. * P≤0.05; ** P≤0.01; *** P≤0.001.

FIG. 9. Exogenous palmitate fatty acid oxidation of 3T3-L1 adipocytes treated with rFGF21, sFGF1-FGF21, and mutants. Figure displayed the exogenous palmitate fatty acid oxidation of 3T3-L1 adipocytes using 0.625 nM of rFGF21, sFGF1-FGF21, G156A, and G189E. All treated groups showed higher OCRs than the control. sFGF1-FGF21 have comparable OCRs (141.5±18.7 pmol/min) to mutant proteins (141.6±6.8 and 150.3±19.0 pmol/min, respectively). G189E has the highest OCR on average. However, there are no statistical differences between sFGF1-FGF21 and mutant-treated cells, indicating that the mutants possess similar metabolic activities as sFGF1-FGF21. Groups were analyzed together using analysis of variance (ANOVA) and post-hoc Bonferroni test (α*). α* for is 0.007143. * p-value <0.05, ** p-value <0.01, *** p-value <0.001. From left to right are control, rFGF21, sFGF1-FGF21, G156A, and G189E.

FIG. 10 shows real-time ATP production rate assay results. ATP production rate of mature 3T3 L1 adipocytes after exposure to four proteins, namely FGF1 (F1), FGF21 (F21), sFGF1-FGF21 (S121), and sFGF1 (S1). sFGF1-FGF21 showed higher activity than the other proteins and the control at a concentration of 0.625 nM. Data is presented as mean±s. d. of total ATP production rate (mitoATP+glycoATP) (n=5). For multiple comparisons with the control, one-way ANOVA with Bonferroni test was performed. * P≤0.05.

FIG. 11. Structure prediction of sFGF1-FGF21 mutants using Colabfold (AlphaFold 2). FIG. 11 depicts a comparison of sFGF1-FGF21 wild-type and its variants (G156A and G189E). There are observable differences in the N-terminus and C-terminus; however, due to low predicted confidence within these regions, no further comments will be made on these differences. Core areas of wild-type and its variants are the same as the predicted structures. The colors of the model correspond to confidence for each region. Blue-colored regions are low in error estimate with high confidence (pLDDT 90-100), light blue is medium-high confidence (pLDDT 70-90), yellow is medium-low confidence (pLDDT 50-70), and red is low confidence with high error estimate (pLDDT 0-50).

FIGS. 12A-12D. Purification of sFGF1-FGF21 mutants without PMSF. Chromatogram of G156A expressed in BL21Star (DE3) using Ni²⁺-Sepharose column (FIG. 12A). Protein was shown to elute at 100 nM IMD and confirmed through an SDS-PAGE run after purification. Chromatogram of G189E expressed in BL21Star (DE3) and purified using Ni²⁺-Sepharose column (FIG. 12B). Protein was suspected to elute at 100 mM IMD and confirmed with SDS-PAGE. Both 100 mM fractions were concentrated, the buffer was exchanged, and protein purity was confirmed via SDS-PAGE. Lanes 1-3 from left to right are G156A, G189E, and the ladder (FIG. 12C). His-tagged sFGF1-FGF21 mutants were confirmed via western blot using anti-His antibody. Lanes 1-3 from left to right are G156A, G189E, and the ladder (FIG. 12D), showing that the obtained products were His-tagged sFGF1-FGF21 mutants G156A and G189E.

FIGS. 13A-13D. Far-UV circular CD and intrinsic fluorescence spectra of sFGF1-FGF21 and mutants. FIG. 13A displayed the circular dichroism spectra of sFGF1-FGF21, G156A, and G189E in molar ellipticity. sFGF1-FGF21 and mutants all have one negative peak around 208-211 nm and a positive peak around 228 nm, consistent with the β-trefoil structure. FIG. 13B showed the intrinsic fluorescence of sFGF1-FGF21, G156A, and G189E, which are tyrosine emissions at 308 nm, indicating correct tertiary folding of sFGF1-FGF21 and mutants. sFGF1-FGF21 is light gray, G156A is gray and G189E is black. FIG. 13C showed G156A tryptophan quenching. The fluorescence trace on the left represents the folded protein with tyrosine peaks at 308 nm, and the trace on the right represents the unfolded protein with tryptophan peaks at 350 nm. FIG. 13D showed G189E tryptophan quenching. The fluorescence trace on the left represents the folded protein with tyrosine peaks at 308 nm, and the trace on the right represents the unfolded protein with tryptophan peaks at 350 nm.

FIGS. 14A-14B. Thermal and chemical stability of sFGF1-FGF21 and mutants. The unfolding process of sFGF1-FGF21, G156A, and G189E using DSC was shown in FIG. 14A. sFGF1-FGF21 showed a Tm around 65.92° C. (±0.1° C.), as reported in Example 1. G156A and G189E showed similar Tm around 65.61° C. (±0.2° C.) and 65.23° C. (±0.1° C.), respectively. FIG. 14B showed the chemical stability of sFGF1-FGF21, G156A, and G189E in denaturant (8 M urea). Cm of sFGF1-FGF21 at 3.26 M (±4.50%). G156A and G189E are both at 3.81 M (±2.02% and 12.9%, respectively). sFGF1-FGF21 is light gray, G156A is gray and G189E is black.

FIGS. 15A-15C. Isothermogram of sFGF1-FGF21 and mutants. The panels showed affinities to heparin of sFGF1-FGF21 (FIG. 15A, as reported in Example 1), G156A (FIG. 15B), and G189E (FIG. 15C). All proteins showed no binding affinity to heparin.

FIG. 16. Limited time-dependent trypsin digestion assay sFGF1-FGF21 and mutants. The figure showed the proteolysis proneness of sFGF1-FGF21 (S121) and mutants using trypsin concentration at 2.15×10⁻⁹ M or 0.00005 mg/mL at 37° C. for 0-80 minutes. Gels were scanned using densitometric scanning software to convert the protein bands into quantified pixel counts. The percentage of digested protein was calculated and graphed (data not shown). As reported in Example 1, sFGF1-FGF21 was rapidly digested protein with only 4.24% (±3.47%) of protein left undigested. Similar to the wild-type, G156A and G189E were not resistant to trypsin digestion throughout the digestion period, leaving 2% (±2.46% and 2.05%, respectively) of protein left undigested after 80 minutes. sFGF1-FGF21 is light gray, G156A is gray and G189E is black.

FIG. 17. The binding curve of ANS to sFGF1-FGF21 and mutants. The figure shows the binding between said proteins to ANS. 520 nm fluorescence wavelength was used to determine the presence of solvent-exposed hydrophobic pockets within the listed proteins. RFU of sFGF1-FGF21 is at 130 nM of ANS (82.14±1.88 RFU), as reported in Example 1. G156A-ANS binding peaks at 140 nM of ANS (93.39±1.27 RFU), similar to the wild-type. In contrast, G189E-ANS is at 200 nM of ANS (138.97±2.60 RFU), higher than both sFGF1-FGF21 and G156A. sFGF1-FGF21 is light gray, G156A is black and G189E is gray.

FIGS. 18A-18C. Stability of sFGF1-FGF21 mutants over time. FIGS. 18A-18C show the stability of G156A and G189E in storage over nine days at three different temperatures (−20° C., 4° C., 25° C.). All samples were from the sample batch and in the same storage conditions. The G156A in working buffer only without PMSF storage conditions degraded slowly at 25° C. and remained mostly intact at −20° C. and 4° C. storage temperature (FIG. 18A). This trend of degradation was quantified from gels using densitometric scan software and compared to each other. G189E in working buffer only without PMSF storage conditions degraded rapidly at 25° C. and remained mostly intact at −20° C. and 4° C. storage temperature (FIG. 18B). Circle is the temperature of −20° C., triangle is the temperature of 4° C., and square is the temperature of 25° C. This trend of degradation was quantified from gels using densitometric scan software and compared to each other. When compared to sFGF1-FGF21 (S121), G156A showed higher stability over time than sFGF1-FGF21 and G189E, notably at the 25° C. storage condition (FIG. 18C).

FIGS. 19A-19E. Cell proliferation assays using NIH/3T3 induced by wtFGF1, sFGF1-FGF21, and mutants. FIGS. 19A-19E demonstrated the cell counts of NIH/3T3 after cells were incubated with the four proteins at different concentrations (0-3.125 nM) for 24 hours and counted by using Hoechst 33342 fluorescent dye in BioTek Cytation 5 (Agilent, USA). FIG. 19A: wtFGF1 (F1) showed dose-dependent cell proliferative activities on fibroblasts with higher concentrations of wtFGF1 proliferate more fibroblasts. FIG. 19B: sFGF1-FGF21 (S121) showed higher proliferative activities at lower concentrations, peaks at 0.625 nM, and lower activities at higher concentrations. FIGS. 19C and 19D: Both G156A and G189E followed the same behaviors and had high mitogenic activities at 0.625 nM. FIG. 19E: all proteins were compared together by ANOVA single factor and Bonferroni post-hoc test (α*). However, there are no statistical differences between 0.625 nM of sFGF1-FGF21 compared to G156A, G189E, and wtFGF1. From left to right are control, sFGF1-FGF21, G156A, G189E, and wtFGF1. α*=0.00263. p-value <0.05 is *, <0.01 is **, <0.001 is***.

### DETAILED DESCRIPTION

The present invention provides fusion proteins comprising a fibroblast growth factor 1 (FGF1) peptide and a fibroblast growth factor 21 (FGF21) peptide. Pharmaceutical compositions and wound dressings comprising the fusion proteins, polynucleotides encoding the fusion proteins, and methods of using the fusion proteins to treat a condition in a subject are also provided.

The present inventors have created a novel FGF21 analog with improved stability and metabolic activity as compared to wild-type FGF21. This FGF21 analog is a fusion protein in which an FGF21 peptide derived from the C-terminal end of FGF21 is fused to an FGF1 peptide, and it is referred to herein as “sFGF1-FGF21”. The FGF1 peptide used in the fusion protein may be super FGF1 (sFGF1), which is a variant of wild-type human FGF1 that comprises five specific mutations (i.e., Q41P, S48L, H94S, K113N, and R123E) that make it hyperstable and enhance its heparin-independent cell proliferation activity, shelf-life, and resistance to proteolysis^{21,22} (see US Patent Publication No. 2019/0284252). The FGF21 peptide used in the fusion protein is a C-terminal fragment of the full-length FGF21 protein. As is demonstrated in the Examples, sFGF1-FGF21 has enhanced thermal stability and chemical stability in urea as compared to both wild-type FGF1 and full-length FGF21. sFGF1-FGF21 has a similar core structure to that of sFGF1, which is compact and less flexible than that of FGF1. This observation is consistent with the secondary and tertiary structure of the fusion protein, which were assessed via circular dichroism, intrinsic fluorescence, extrinsic fluorescence ANS-binding assay, and protein structure prediction. sFGF1-FGF21 also has more potent metabolic activities as compared to FGF21 (i.e., it more potently increases fat cell glucose uptake and fatty acid oxidation) and more potent mitogenic activity as compared to sFGF1.

Additionally, the inventors have generated two variants of the sFGF1-FGF21 fusion protein that each comprise a specific substitution mutation within the FGF21 peptide. One variant comprises the mutation G169A (with reference to wild-type FGF21 of SEQ ID NO: 3, found at G141 in the truncated FGF21 of SEQ ID NO: 4) while the other comprises the mutation G202E (with reference to the wild-type FGF21 sequence in SEQ ID NO: 3 or at position G174 with reference to the truncated FGF21 provided as SEQ ID NO: 4. The inventors determined that the G169A variant is more stable than the wild-type version of sFGF1-FGF21.

Please note that the FGF21 mutations are referred to in the Detailed Description and the Claims based on their position within the full-length FGF21 protein sequence of SEQ ID NO: 3, but are referred to in the Drawings, Brief Description of the Drawings, and Examples based on their position within the fusion protein sequence of SEQ ID NO: 14. A comparison of these residue labels is provided in Table 1 below.

TABLE 1

| Position in | Position in | Position in SEQ ID NO: 4 |
|---|---|---|
| SEQ ID NO: 3 | SEQ ID NO: 14 | (FGF21 lacking signal |
| Full length FGF21 | sFGF1-FGF21 fusion | sequence) |
| G169 | G156 | G141 |
| G202 | G189 | G174 |

### Fusion Proteins:

In a first aspect, fusion proteins comprising a fibroblast growth factor 1 (FGF1) peptide linked to a fibroblast growth factor 21 (FGF21) peptide are provided.

The terms “protein,” “polypeptide,” and “peptide” are used interchangeably herein to refer to polymers of amino acids connected by peptide bonds between the alpha-amino and carboxy groups of adjacent amino acids. Proteins may be modified to include non-amino acid moieties (e.g., via acetylation or glycosylation) and may include amino acid analogs. The terms “protein” and “polypeptide” are often used in reference to relatively large polypeptides, whereas the term “peptide” is often used in reference to small polypeptides, but usage of these terms in the art overlaps.

A “fusion protein” is an engineered protein created by joining together two or more polynucleotides that originally encoded separate proteins such that the two or more polynucleotides are transcribed and translated as a single unit to produce a single protein. The two proteins may be linked by a peptide bond directly or a linker may be included such that the fusion protein structure is peptide 1-linker-peptide 2. Suitable peptide linkers are known in the art and may be 1-20 amino acids in length and may include residues that allow for protein folding of the two separate subunit peptides contained in the fusion protein. Flexible linkers often include serine and glycine residues.

Fibroblast growth factor 21 (FGF21) is a protein that is secreted by the liver that regulates glucose and lipid metabolism. The FGF21 peptide used in the fusion proteins of the present invention may be from any organism. However, in preferred embodiments, the FGF21 peptide is derived from human FGF21.

In some embodiments, the FGF21 peptide comprises the full-length, wild-type, human FGF21 protein of SEQ ID NO: 3. As used herein the term “wild-type” refers to the typical form of a protein that occurs in nature. In other embodiments, the FGF21 peptide comprises a variant of SEQ ID NO: 3. As used herein, the term “variant” refers to a protein that has an amino acid sequence that differs from a reference protein, such as a wild-type protein. A variant may have one or more amino acid insertions, deletions, or substitutions relative to the reference protein or may be a fragment of the reference protein. In some embodiments, the FGF21 peptide is a variant that has at least 75%, 80%, 85%, 90%, 95%, 96%, 97%, 98%, or 99% sequence identity to SEQ ID NO: 3 or a fragment thereof. The fragments of SEQ ID NO: 3 useful in the fusion protein may be those provided herein as SEQ ID NOs: 4-9 or homologs thereof having at least 75%, 80%, 85%, 90%, 95%, 96%, 97%, 98%, or 99% sequence identity to at least one of SEQ ID NOs: 4-9.

“Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100. Protein and nucleic acid sequence identities can be evaluated using the Basic Local Alignment Search Tool (“BLAST”), which is well known in the art (Karlin and Altschul, Methods for assessing the statistical significance of molecular sequence features by using general scoring schemes. Proc. Natl. Acad. Sci. USA (1990) 87:2267-2268; Altschul et al. Gapped BLAST and PSI-BLAST: a new generation of protein database search programs. Nucl. Acids Res. (1997) 25:3389-3402). The BLAST programs identify homologous sequences by identifying similar segments between a query amino acid or nucleic acid sequence and a test sequence, which is preferably obtained from a protein or nucleic acid sequence database. The BLAST programs can be used with the default parameters or with modified parameters provided by the user.

As used herein, a “fragment” is a portion of a polypeptide that is identical in sequence to, but shorter in length than, the full-length polypeptide. For example, a fragment may comprise at least 20, 25, 30, 40, 50, 60, 70, 80, 90, 100, 150, 250, or 500 contiguous amino acid residues of a full-length polypeptide. Fragments may be preferentially selected from certain regions of a polypeptide. A fragment may include an N-terminal truncation, a C-terminal truncation, or both an N-terminal and C-terminal truncation relative to the full-length polypeptide. Preferably, peptide fragments used with the present invention are functional fragments. As used herein, a “functional fragment” is a fragment that retains at least 20%, 40%, 60%, 80%, or 100% of the biological activity of the active form of the protein.

The full-length, wild-type, human FGF21 protein (SEQ ID NO: 3) is 209 amino acids in length. Thus, a fragment of FGF21 may comprise 20 to 208 amino acids. In some embodiments, the FGF21 fragment is 30 to 200 amino acids, 35 to 150 amino acids, 40 to 100 amino acids, or 45 to 55 amino acids in length. The FGF21 fragments tested in the Examples are 49 amino acids in length. Thus, in specific embodiments, the FGF21 fragments are 49 amino acids in length.

The full-length FGF21 protein comprises an N-terminal signal sequence (i.e., residues 1-28 of SEQ ID NO: 3) that directs the protein to be secreted from the cell by targeting it to the endoplasmic reticulum. This signal sequence is cleaved off during protein maturation and is not included in the secreted, mature FGF21 protein (SEQ ID NO: 4). Removal of this signal sequence allows for FGF21 to properly interact with its receptor complex. Thus, in preferred embodiments, the FGF21 peptide does not include the N-terminal signal sequence. Notably, in the experiments described in the Examples, the inventors used a recombinant FGF21 that lacks the signal sequence and includes a 6× polyhistidine tag (SEQ ID NO: 5) for comparison with their fusion proteins.

As is described in the Examples, the inventors utilized a C-terminal fragment (i.e., a fragment comprising the C-terminus) of FGF21 in their fusion protein. Thus, in some embodiments, the FGF21 peptide is a C-terminal fragment of FGF21. Specifically, the inventors utilized a C-terminal fragment (SEQ ID NO: 6) comprising residues 161-209 of the full-length FGF21 protein of SEQ ID NO: 3. Thus, in some embodiments, the FGF21 peptide has at least 75%, 80%, 85%, 90%, 95%, 96%, 97%, 98%, or 99% sequence identity to SEQ ID NO: 6. In some embodiments, the FGF21 peptide comprises or consists of SEQ ID NO: 6.

The FGF21 peptide used in the fusion protein may have at least 75%, 80%, 85%, 90%, 95%, 96%, 97%, 98%, or 99% sequence identity to the full-length, wild-type FGF21 protein of SEQ ID NO: 3 or to any fragment thereof, and it may comprise one or more substitution mutation relative to SEQ ID NO: 3. A “substitution mutation” or “substitution” is a replacement of one amino acid in a reference sequence with a different amino acid. A substitution may be conversative (i.e., a replacement with an amino acid that has similar properties) or radical (i.e., a replacement with an amino acid that has different properties). For example, an FGF21 variant may comprise 1, 2, 3, 4, 5, 6, 7, 8, 9, 10, or more substitutions relative to SEQ ID NO: 3.

In the Examples, the inventors made substitution mutations in the FGF21 peptide portion of their fusion protein and tested the effect of these mutations on fusion protein stability. Specifically, they made mutations in residues G169 and G202 of FGF21 (numbered with respect to the full-length FGF21 protein sequence of SEQ ID NO: 3). Thus, in some embodiments, the FGF21 peptide comprises at least one substitution mutation at a residue selected from residue G169 and residue G202. In specific embodiments, the substitution mutation at residue G169 is G169A and/or the substitution mutation at residue G202 is G202E.

In the Examples, the inventors determined that residue R150 of SEQ ID NO: 14 in the FGF21 portion of the fusion protein is likely a cleaving target during proteolysis and suggest that providing a mutation at residue R150 could improve the fusion protein's resistance to proteolysis. Thus, in some embodiments, the FGF21 peptide comprises at least one mutation at the residue R150. The mutants provided here may also affect degradation at position 150 of FGF21. The inventors do not wish to be bound by any single theory at this time.

As is noted above, the fusion proteins tested in the Examples comprised a C-terminal fragment of FGF21 consisting of residues 161-209 of the full-length FGF21 protein. In addition to the wild-type FGF21 C-terminal fragment of SEQ ID NO: 6, the inventors also tested variants of this fragment comprising the G169A mutation (SEQ ID NO: 7) or the G202E mutation (SEQ ID NO: 8) in their fusion protein, and they envision that this FGF21 fragment may also comprise both the G169A mutation and the G202E mutation (SEQ ID NO: 9). Thus, in some embodiments, the FGF21 peptide is a C-terminal fragment having at least 75%, 80%, 85%, 90%, 95%, 96%, 97%, 98%, or 99% sequence identity to SEQ ID NO: 7, SEQ ID NO: 8, or SEQ ID NO: 9. In some embodiments, the FGF21 peptide comprises or consists of SEQ ID NO: 7, SEQ ID NO: 8, or SEQ ID NO: 9.

Fibroblast growth factor 1 (FGF1) is a mitotic protein that plays a critical role in wound healing. The FGF1 peptide used in the fusion proteins of the present invention may be from any organism. However, in preferred embodiments, the FGF1 peptide is derived from human FGF1.

The FGF1 peptide used in the fusion proteins has at least 75%, 80%, 85%, 90%, 95%, 96%, 97%, 98%, or 99% sequence identity to SEQ ID NO: 1, which is the sequence of the full-length, wild-type, human FGF1 protein. In some embodiments, the FGF1 peptide comprises the full-length, wild-type, human FGF1 protein of SEQ ID NO: 1. In other embodiments, the FGF1 peptide is a variant of SEQ ID NO: 1.

In previous work described in U.S. Patent Publication No. US 2019/0284252, which is hereby incorporated by reference in its entirety, the inventors generated an engineered FGF1 variant with enhanced thermal stability, resistance to proteolytic activity, and heparin-independent bioactivity as compared to wild-type FGF1. This hyperstable FGF1 variant comprises the mutations Q41P, S48L, H94S, K113N, and R123E relative to SEQ ID NO: 1 and is referred to herein as “super FGF1” or “sFGF1”. The amino acid sequence of sFGF1 is provided as SEQ ID NO: 2. The inventors used this hyperstable variant in the fusion proteins tested in the Examples. Thus, in some embodiments, the FGF1 peptide comprises at least one substitution mutation at a residue that is mutated in sFGF1, namely in at least one of residues Q41, S48, H94, K113, and R123. The FGF1 variant may comprise substitution mutation(s) at 1, 2, 3, 4, or all 5 of these residues. In these embodiments, (a) the substitution mutation at residue Q41 is selected from Q41P, Q41F, Q41M, Q41Y, Q41W, Q41I, Q41L, Q41V, and Q41A; (b) the substitution mutation at residue S48 is selected from S48L, S48A, S48V, S48P, S48T, S48M, S48I, S48F, S48Y, and S48W; (c) the substitution mutation at residue H94 is selected from H94S, H94T, H94K, H94R, and H94Y; (d) the substitution mutation at residue K113 is selected from K113N, K113Q, K113S, K113T, K113R, and K113Y; and/or (e) the substitution mutation at residue R123 is selected from R123E and R123D. In some embodiments, the FGF1 variant comprises one or more of the specific substitution mutations made in sFGF1, namely it comprises one of more of Q41P, S48L, H94S, K113N, and R123E. The hFGF1 variant may comprise 1, 2, 3, 4 or all 5 of these specific substitution mutations. In some embodiments, the FGF1 peptide has at least 75%, 80%, 85%, 90%, 95%, 96%, 97%, 98%, or 99% sequence identity SEQ ID NO: 2. In some embodiments, the FGF1 peptide comprises or consists of SEQ ID NO: 2.

The FGF1 peptides and FGF21 peptides described herein may include additional modifications (i.e., substitutions, insertions, deletions) other than those explicitly described. Modifications based on the alternative amino acid residues that occur at a corresponding position in an FGF homolog from another species are expected to conserve protein function. Accordingly, the additional modifications may be selected using a sequence alignment of a particular FGF polypeptide with the corresponding FGF polypeptide from another species.

In some embodiments, the fusion protein comprises sFGF1 (SEQ ID NO: 2) and the specific wild-type C-terminal fragment of FGF21 that was tested in the examples (SEQ ID NO: 6) or a variant thereof comprising the G169A mutation (SEQ ID NO: 7), the G202E mutation (SEQ ID NO: 8), or both the G169A mutation and the G202E mutation (SEQ ID NO: 9). For example, in some embodiments, the FGF1 peptide comprises SEQ ID NO: 2 and the FGF21 peptide comprises SEQ ID NO: 6; the FGF1 peptide comprises SEQ ID NO: 2 and the FGF21 peptide comprises SEQ ID NO: 7; the FGF1 peptide comprises SEQ ID NO: 2 and the FGF21 peptide comprises SEQ ID NO: 8; or the FGF1 peptide comprises SEQ ID NO: 2 and the FGF21 peptide comprises SEQ ID NO: 9.

In the fusion proteins tested in the Examples, the C-terminus of the FGF1 peptide is directly linked to the N-terminus of the FGF21 peptide. Thus, in some embodiments, the components of the fusion protein are arranged in the following order from N-terminus to C-terminus: FGF1 peptide-FGF21 peptide. In some embodiments, the FGF1 peptide and the FGF21 peptide are linked together directly (i.e., without a linker peptide). For example, the fusion protein may comprise: sFGF1 linked directly to the wild-type FGF21 C-terminal fragment (SEQ ID NO: 10), sFGF1 linked directly to the G169A FGF21 C-terminal fragment (SEQ ID NO: 11), sFGF1 linked directly to the G202E FGF21 C-terminal fragment (SEQ ID NO: 12), or sFGF1 linked directly to the G169A/G202E FGF21 C-terminal fragment (SEQ ID NO: 13). In some embodiments, the fusion protein has at least 75%, 80%, 85%, 90%, 95%, 96%, 97%, 98%, or 99% sequence identity to SEQ ID NO: 10, SEQ ID NO: 11, SEQ ID NO: 12, or SEQ ID NO: 13. In some embodiments, the fusion protein comprises or consists of SEQ ID NO: 10, SEQ ID NO: 11, SEQ ID NO: 12, or SEQ ID NO: 13. In other embodiments, the components of the fusion protein are arranged in the reverse order: FGF21 peptide-FGF1 peptide.

It is possible that including a linker peptide in the fusion protein (i.e., between the FGF1 peptide and the FGF21 peptide) could enhance the biological activity of one or both peptide components by allowing them to move more freely relative to one another. Thus, in some embodiments, the FGF1 peptide is linked to the FGF21 peptide via a linker peptide. As used herein, the term “linker peptide” refers to a peptide that links two segments of a fusion protein together via peptide bonds, allowing the two segments to be translated as a single polypeptide. A linker peptide may comprise 1, 2, 3, 4, 5, 6, 7, 8, 9, 10, 11, 12, 13, 14, 15, 16, 17, 18, 19, 20, or more amino acid residues. A linker peptide may comprise any amino acid sequence that does not substantially hinder the function of the two polypeptide segments that it links. In some embodiments, the linker is flexible such that it has no required fixed structure in solution and the polypeptide segments that it links are free to move relative to one another. Preferred amino acid residues for flexible linker sequences include glycine, alanine, serine, threonine, lysine, arginine, glutamine, and glutamic acid.

In some embodiments, the fusion proteins further comprise a protein tag. A “tag” is a peptide that is included in a fusion protein to serve a particular function. Suitable tags for use in the fusion proteins of the present invention include, without limitation, (a) affinity tags for protein purification (e.g., chitin binding protein (CBP), maltose binding protein (MBP), Strep, glutathione-S-transferase (GST), and polyhistidine tags), (b) solubilization tags (e.g., thioredoxin (TRX), poly (NANP), MBP, GST), (c) epitope tags for antibody-based detection (e.g., ALFA-tag, V5-tag, Myc-tag, HA-tag, Spot-tag, T7-tag and NE-tag), (d) fluorescent tags (e.g., green fluorescent protein (GFP), red fluorescent protein (RFP)) and enzymatic tags (e.g., horseradish peroxidase, alkaline phosphatase, beta-galactosidase, glucose-6-phosphatase, acetylcholinesterase) for visual detection, (e) targeting tags that target the fusion proteins to a particular site or tissue type within the body (e.g., fibrin-, thrombin-, fibronectin-, or collagen-binding peptide), and (f) cell penetrating peptides that deliver the fusion proteins into cells. The tag may be included on the N-terminus of the fusion protein, on the C-terminus of the fusion protein, or between the FGF1 peptide and the FGF21 peptide. Optionally, the tag may be separated from the other components of the fusion protein by a protease cleavage site to allow for removal of the tag after it has served its purpose.

The fusion proteins tested in the Examples comprised a 6× polyhistidine tag (i.e., a protein tag comprising six histidine residues) to allow them to be purified via nickel affinity chromatography. Thus, in some embodiments, the protein tag is a polyhistidine tag (i.e., a protein tag comprises multiple histidine residues). An example of one such fusion protein is provided as SEQ ID NO: 14, which includes, from N-terminus to C-terminus: a 6× polyhistidine tag, sFGF1, and a wild-type C-terminal fragment of FGF21. In some embodiments, the fusion protein has at least 75%, 80%, 85%, 90%, 95%, 96%, 97%, 98%, or 99% sequence identity to SEQ ID NO: 14. In some embodiments, the fusion protein comprises or consists of SEQ ID NO: 14.

### Pharmaceutical Compositions

In a second aspect, pharmaceutical compositions comprising a fusion protein described herein and a pharmaceutically acceptable carrier are provided.

As used herein, the term “carrier” refers to a substance used to deliver an agent to a targeted site within the body. A “pharmaceutically acceptable carrier” is a carrier that is compatible with the other ingredients of the composition and that is not deleterious to the subject to which the composition is to be administered. Pharmaceutically acceptable carriers include, but are not limited to, excipients, diluents (e.g., Tris-HCl, acetate, phosphate), preservatives (e.g., thimerosal, benzyl alcohol, parabens), solubilizing agents (e.g., glycerol, polyethylene glycerol), emulsifiers, liposomes, nanoparticles, and adjuvants. Pharmaceutically acceptable carriers may be aqueous or non-aqueous solutions, suspensions, or emulsions. In some embodiments, the carrier is a hydrogel (i.e., a gel in which the liquid component is water). In specific embodiments, the carrier is PEG-co-PAA or a derivative thereof. In some embodiments, the carrier allows for time-dependent release of the agent.

The pharmaceutical compositions of the present invention may further include additives such as albumin or gelatin to prevent absorption to surfaces, detergents (e.g., Tween 20, Tween 80, Pluronic F68, bile acid salts), antioxidants (e.g., ascorbic acid, sodium metabisulfite), bulking substances or tonicity modifiers (e.g., lactose, mannitol).

### Polynucleotides:

In a third aspect, polynucleotides encoding a fusion protein described herein are provided.

The terms “polynucleotide,” “nucleic acid,” and “oligonucleotide” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages (e.g., phosphoroamidate linkages, phosphorothioate linkages).

In the Examples, the inventors' codon optimized polynucleotides encoding their fusion proteins for expression in Escherichia coli. Thus, in some embodiments, the polynucleotide is codon optimized for expression in a particular cell type. “Codon optimization” is a process used to increase expression of a polynucleotide in a particular cell type by altering the sequence of the polynucleotide to accommodate the codon bias of that cell type. Computer programs for generating codon-optimized sequences for use in a particular cell type are known in the art. In some embodiments, the cell is E. coli.

The sequences of the codon optimized polynucleotides utilized by the inventors to express the fusion proteins tested in the Examples are provided as SEQ ID NO: 15 (sFGF1 linked to the wild-type FGF21 C-terminal fragment), SEQ ID NO: 16 (sFGF1 linked to the G169A FGF21 C-terminal fragment), and SEQ ID NO: 17 (sFGF1 linked to the G202E FGF21 C-terminal fragment). In some embodiments, the polynucleotide has at least 75%, 80%, 85%, 90%, 95%, 96%, 97%, 98%, or 99% sequence identity to SEQ ID NO: 15, SEQ ID NO: 16, or SEQ ID NO: 17. In some embodiments, the polynucleotide comprises or consists of SEQ ID NO: 15, SEQ ID NO: 16, or SEQ ID NO: 17.

The present invention also provides constructs comprising a polynucleotide described herein operably linked to a promoter. As used herein, the term “construct” refers a to recombinant polynucleotide, i.e., a polynucleotide that was formed by combining at least two polynucleotide components from different sources. For example, a construct may comprise the coding region of one gene operably linked to a promoter that is (1) associated with another gene found within the same genome, (2) from the genome of a different species, or (3) synthetic. Constructs can be generated using conventional recombinant DNA methods.

As used herein, the term “promoter” refers to a DNA sequence that defines where transcription of a polynucleotide begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can affect transcription of the polynucleotide. Suitable promoters for use with the present invention include, but are not limited to, constitutive, inducible, temporally regulated, developmentally regulated, chemically regulated, tissue-preferred, and tissue-specific promoters. The promoter may be an animal, plant, bacterial, fungal, or synthetic promoter.

In some embodiments, the construct is a vector. The term “vector” refers to a DNA molecule that is used to carry a particular DNA segment into a host cell. Some vectors are capable of autonomous replication in a host cell (e.g., bacterial vectors that include an origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product. Vectors may also include a reporter gene or a selectable marker gene. Suitable vectors include plasmids (i.e., circular double-stranded DNA molecules) and mini-chromosomes.

### Wound Dressings:

In a fourth aspect, wound dressings comprising a fusion protein or pharmaceutical composition described herein are provided.

As used herein, the term “wound dressing” refers to a product that is applied directly to a wound. The wound dressing may be embedded or implanted with the fusion protein or pharmaceutical composition so that the fusion protein is transferred to the wound when the wound dressing is applied. Examples of suitable wound dressings include, but are not limited to, gauze pads, films, bandages, artificial skin, ointments, creams, sprays, and intradermal injections. The wound dressing may include granules, beads, gels, hydrogels, liquids, and/or pastes that comprise the fusion protein.

### Methods of Treatment:

In a fifth aspect, methods of treating a condition in a subject are provided. The methods comprise administering a fusion protein, pharmaceutical composition, or wound dressing described herein to the subject.

As used herein, “treating” describes something that is done to a subject to combat a condition. Treating may involve controlling the condition, lessening its symptoms or complications, slowing its progression, and/or eliminating it. In preferred embodiments, a therapeutically effective amount of the composition is administered to the subject. The term “therapeutically effective amount” refers to an amount sufficient to achieve one or more desirable treatment outcome. For any active agent, a therapeutically effective amount can be estimated initially in cell culture assays or in an animal model.

As used herein, the term “condition” refers to an illness, injury, disease, or disorder. FGF21 has shown promise as a treatment for metabolic syndromes in animal models^{7,8}. Thus, in some embodiments, the condition is insulin resistance, type 2 diabetes, gestational diabetes, hyperglycemia, obesity, heart disease, or stroke. Additionally, FGF21 has been shown to increase the fatty acid oxidation rate in the liver^{9,10}. Thus, in some embodiments, the condition is nonalcoholic fatty liver disease (NAFLD), nonalcoholic steatohepatitis (NASH), or alcoholic fatty liver disease. FGF1 is known to play a crucial role in wound healing, nerve regeneration, and angiogenesis, which are critical for proper healing after an injury. Thus, in some embodiments, the condition is a wound (e.g., internal, external, chronic, acute, related to diabetes), tissue or organ damage, a fractured bone, a gastric ulcer, ulcerative colitis, a scar, a nerve injury, a burn, hair loss (e.g., alopecia), osteoporosis, a cardiovascular disorder, a cosmetic condition, ischemia (e.g., critical limb ischemia), a retinal disorder, a muscular disorder, an arterial disease, an age-related disorder, osteoarthritis, or acute renal failure.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraoral administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be performed in a single dose or multiple doses and may be continuous or intermittent. In some embodiments, administration is topical, oral, or intravenous.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

### EXEMPLARY EMBODIMENTS

Embodiment 1. A fusion protein comprising: a) a fibroblast growth factor 1 (FGF1) peptide having at least 95% sequence identity to SEQ ID NO: 1; and b) a fibroblast growth factor 21 (FGF21) peptide having at least 95% sequence identity to SEQ ID NO: 3 or a fragment thereof comprising 30-200 amino acid residues, wherein the FGF21 peptide comprises at least one substitution mutation at a residue selected from residues G169 and G202 of SEQ ID NO: 3.

Embodiment 2. The fusion protein of embodiment 1, wherein a) the substitution mutation at residue G169 is G169A; and/or b) the substitution mutation at residue G202 is G202E.

Embodiment 3. The fusion protein of any one of the preceding embodiments, wherein the FGF21 peptide has at least 95% sequence identity to a C-terminal fragment of SEQ ID NO: 3.

Embodiment 4. The fusion protein of embodiment 3, wherein the FGF21 peptide comprises or consists of SEQ ID NO: 7, SEQ ID NO: 8, or SEQ ID NO: 9.

Embodiment 5. A fusion protein comprising: a) an FGF1 peptide having at least 95% sequence identity to SEQ ID NO: 1; and b) an FGF21 C-terminal fragment having at least 95% sequence identity to SEQ ID NO: 6, SEQ ID NO: 7, SEQ ID NO: 8, or SEQ ID NO: 9.

Embodiment 6. The fusion protein of embodiment 5, wherein the FGF21 C-terminal fragment comprises or consists of SEQ ID NO: 6, SEQ ID NO: 7, SEQ ID NO: 8, or SEQ ID NO: 9

Embodiment 7. The fusion protein of any one of the preceding embodiments, wherein the FGF1 peptide comprises at least one substitution mutation at a residue selected from residues Q41, S48, H94, K113, and R123 of SEQ ID NO: 1.

Embodiment 8. The fusion protein of embodiment 7, wherein: a) the substitution mutation at residue Q41 is selected from Q41P, Q41F, Q41M, Q41Y, Q41W, Q41I, Q41L, Q41V, and Q41A;
b) the substitution mutation at residue S48 is selected from S48L, S48A, S48V, S48P, S48T, S48M, S48I, S48F, S48Y, and S48W; c) the substitution mutation at residue H94 is selected from H94S, H94T, H94K, H94R, and H94Y; d) the substitution mutation at residue K113 is selected from K113N, K113Q, K113S, K113T, K113R, and K113Y; e) the substitution mutation at residue R123 is selected from R123E and R123D; or f) any combination of (a)-(e).

Embodiment 9. The fusion protein of embodiment 8, wherein the FGF1 peptide comprises the substitution mutations Q41P, S48L, H94S, K113N, and R123E.

Embodiment 10. The fusion protein of embodiment 9, wherein the FGF1 peptide has at least 95% sequence identity to SEQ ID NO: 2.

Embodiment 11. The fusion protein of embodiment 10, wherein the FGF1 peptide comprises or consists of SEQ ID NO: 2.

Embodiment 12. The fusion protein of any one of the preceding embodiments, wherein the fusion protein comprises from N-terminus to C-terminus: the FGF1 peptide and the FGF21 peptide.

Embodiment 13. The fusion protein of any one of the preceding embodiments, wherein the FGF1 peptide is linked to the FGF21 peptide via a linker peptide.

Embodiment 14. The fusion protein of any one of the preceding embodiments, wherein: a) the FGF1 peptide comprises SEQ ID NO: 2 and the FGF21 peptide comprises SEQ ID NO: 6; b) the FGF1 peptide comprises SEQ ID NO: 2 and the FGF21 peptide comprises SEQ ID NO: 7; c) the FGF1 peptide comprises SEQ ID NO: 2 and the FGF21 peptide comprises SEQ ID NO: 8; or d) the FGF1 peptide comprises SEQ ID NO: 2 and the FGF21 peptide comprises SEQ ID NO: 9.

Embodiment 15. The fusion protein of embodiment 14, wherein the fusion protein has at least 95% sequence identity to SEQ ID NO: 10, SEQ ID NO: 11, SEQ ID NO: 12, or SEQ ID NO: 13.

Embodiment 16. The fusion protein of any one of the preceding embodiments, further comprising a protein tag.

Embodiment 17. The fusion protein of embodiment 16, wherein the protein tag is a polyhistidine tag.

Embodiment 18. The fusion protein of embodiment 17, wherein the fusion protein has at least 95% sequence identity to SEQ ID NO: 14.

Embodiment 19. A pharmaceutical composition comprising the fusion protein of any one of the preceding embodiments and a pharmaceutically acceptable carrier.

Embodiment 20. A polynucleotide encoding the fusion protein of any one of embodiments 1-18.

Embodiment 21. The polynucleotide of embodiment 20, wherein the polynucleotide is codon optimized for expression in a cell.

Embodiment 22. The polynucleotide of embodiment 21, wherein the cell is Escherichia coli.

Embodiment 23. The polynucleotide of embodiment 22, wherein the polynucleotide comprises SEQ ID NO: 15, SEQ ID NO: 16, or SEQ ID NO: 17.

Embodiment 24. A construct comprising the polynucleotide of any one of embodiments 20-23 operably linked to a promoter.

Embodiment 25. A wound dressing comprising the fusion protein of any one of embodiments 1-18 or the pharmaceutical composition of embodiment 19.

Embodiment 26. A method of treating a condition in a subject, the method comprising administering the fusion protein of any one of embodiments 1-18, the pharmaceutical composition of embodiment 19, or the wound dressing of embodiment 25 to the subject.

Embodiment 27. The method of embodiment 26, wherein the condition is type 2 diabetes, insulin resistance, gestational diabetes, hyperglycemia, obesity, heart disease, stroke, nonalcoholic fatty liver disease (NAFLD), nonalcoholic steatohepatitis (NASH), alcoholic fatty liver disease, a wound, tissue or organ damage, a fractured bone, a gastric ulcer, ulcerative colitis, a scar, a nerve injury, a burn, hair loss, osteoporosis, a cardiovascular disorder, a cosmetic condition, ischemia, a retinal disorder, a muscular disorder, an arterial disease, an age-related disorder, osteoarthritis, or acute renal failure.

Embodiment 28. The method of embodiment 26 or 27, wherein the subject is a human.

Embodiment 29. The method of any one of embodiments 26-28, wherein administration is topical, oral, or intravenous.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

### Example 1. Designing, Cloning, Over-Expression, Purification, and Structural Characterization of a Novel Chimeric sFGF1-FGF21 Protein with Enhanced Stability

The metabolic activities of fibroblast growth factor (FGF) on glucose and fatty acid metabolisms in humans make FGF21 a promising candidate for therapeutical development as a treatment for metabolic diseases like obesity and diabetes. However, due to the unstable nature of FGF21 and its short-acting half-life, translating FGF21 into practical therapeutics is challenging. This project aimed to design and produce a novel FGF1-FGF21 fusion protein, called sFGF1-FGF21, and characterize its structural features through biophysical techniques. Successfully produced sFGF1-FGF21 were hypothesized to retain most of sFGF1's biophysical features and stability.

Indeed, sFGF1-FGF21 showed a similar secondary structure through CD spectra with a positive peak at 228 nm and a negative peak at 207 nm, indicating a β-sheet core like wtFGF1 and sFGF1. The fusion protein exhibited tyrosine intrinsic fluorescence at 306-309 nm, with a quenched tryptophan emission like wtFGF1-FGF21. The fusion protein is thermally stable with a Tm of 65.92±0.1° C. Likewise, sFGF1-FGF21 has high chemical resistance to urea unfolding with Cm of 3.26±0.045 M. sFGF1-FGF21 also exhibited high core compactness like sFGF1 through ANS binding assay with maximum fluorescence intensity at 130 nM of ANS (82.14±1.88 RFU), less than other proteins. sFGF1-FGF21 also showed no binding affinity to heparin, like sFGF1. However, due to the long and flexible FGF21 C-terminus, the fusion protein is prone to protease cleavages through limited trypsin digestion assay. sFGF1-FGF21 is also prone to degradation in storage over time. These undesirable traits may be due to the Ariginine¹⁵⁰ residue in this region, which is a likely a target for proteolysis and degradation. The findings achieved the aim of producing sFGF1-FGF21 that retains the beneficial features of sFGF1 and has higher thermal and chemical stability than rFGF21. In conclusion, the results showed a successful proof-of-concept to fuse paracrine and endocrine FGFs to enhance the stability of the design for the future development of FGF21-based therapeutics.

Herein, we develop a new model of FGF21 analog: an FGF1-FGF21 chimeric protein with an optimized sequence that possesses enhanced stability inherited from sFGF1. Using a common bacterial expression system, we were able to produce the protein at high purity and yield. Further experiments using biophysical techniques showed that the chimeric protein does possess higher thermal and chemical stability than wild-type counterparts and similar structural characteristics like sFGF1. Our results here create an FGF1-FGF21 chimera with increased stability.

### Results and Discussion

Design, Overexpression, Purification, and Authentication of a Soluble sFGF1-FGF21

The new model of fusion protein FGF1-FGF21, also known as sFGF1-FGF21, combines the hyper-stable super hFGF1 (sFGF1) with the C-terminus of FGF21. We utilized the 6× his-tagged into the design of this chimera protein (~22.836) kDa) expressed in BL21Star (DE3) bacterial strain, which has a reducing cytoplasm [34]. sFGF1-FGF21 was designed (Genscript, USA) into a pET-21a+ vector.

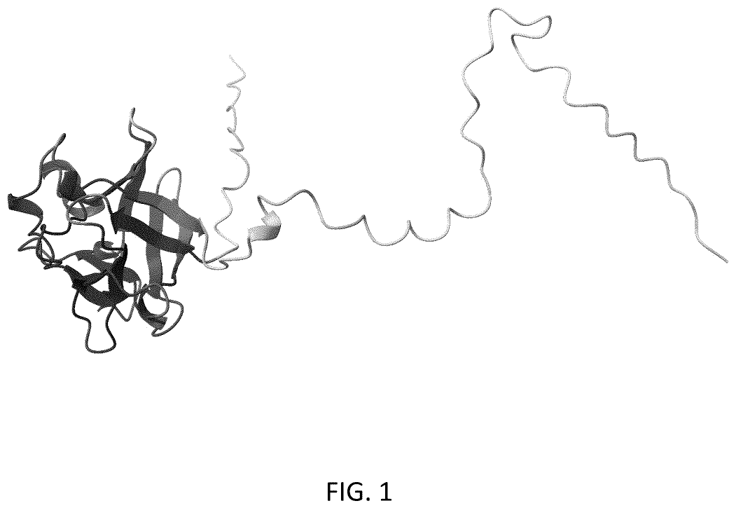

ColabFold (AlphaFold 2) was used to predict the structure of the chimera protein [35]. The structure prediction also confirmed that the addition of the 6×-histidine tag does not alter the conformation of the protein too drastically [35]. FIG. 1 shows that the sFGF1-FGF21 predicted structure has characteristics and features similar to those of the reported sFGF1 [36]. Similar to sFGF1 and hFGF1, a canonical β-trefoil core is seen in the expected structure of sFG1-FGF21 [36]. The C-terminus of sFGF1-FGF21 is similar to rFGF21, which is long and flexible and may be disordered based on both the low pLDDT score (between 50-70) for this region in the chimeric protein's structure prediction and on the PONDR analysis of sFGF1-FGF21, which showed high disordered-ness in the C-terminus.

Transformations of sFGF1-FGF21 were made in XL 10 Gold and BL21Star (DE3). rFGF21 has a disulfide bridge, which cannot be formed in many E. coli systems like BL21 Star (DE3) due to having many reductases, making their cytoplasm “reducing environments” and not suitable to produce disulfide-bonded protein [14, 37]. However, the disulfide bridge is not at the C-terminus. hFGF1 and sFGF1 do not have any disulfide bridges. Therefore, sFGF1-FGF21 does not require specialized Rosetta-origami cells to catalyze disulfide bond formation in the cytoplasm and, thus, can use a model bacterial expression system like BL21Star (DE3). Indeed, sFGF1-FGF21 transformed well in BL21Star (DE3) and produced an ample number of colonies. Similarly, sFGF1 was also transformed into BL21Star (DE3) and made a high quantity of colonies.

For the generation of more plasmid and verification of plasmid identity, sFGF1-FGF21 was transformed into XL 10 Gold. The chimera protein-containing plasmid was shown to transform well in this cell line. Culturing transformed XL 10 Gold and subsequent plasmid isolation via salt-out procedure showed adequate quantity and quality of sFGF1-FGF21 plasmid after agarose gel electrophoresis. The sample was sent for DNA sequencing (Plasmidsaurus, USA). The result showed that the plasmid contains the sFGF1-FGF21 gene (SEQ ID NO: 18), indicating that the gene construction and transformation processes were successful.

sFGF1-FGF21 was overexpressed in BL21Star (DE3) cells on a small scale (10 mL of cell culture) after a successful transformation with discernable colonies. Three separate random colonies of sFGF1-FGF21-containing bacteria were chosen to culture in the LB medium. Once the OD of the culture reached 0.6-0.8, the culture was incubated with one mM Isopropyl ß-D-1-thiogalactopyranoside (IPTG) to induce expression for 5 hours, then harvested and lysed using Sonifier (Branson, USA). Lysate was run in a tabletop centrifuge at the speed of 13,000 rpm and analyzed on Sodium dodecyl sulfate-polyacrylamide gel electrophoresis (SDS-PAGE, 15%) gel to confirm the existence of induced sFGF1-FGF21. From the gel result, we observed that colony 1 has the most sFGF1-FGF21 expression. Thus, colony one was chosen for large-scale overexpression of the chimeric protein.

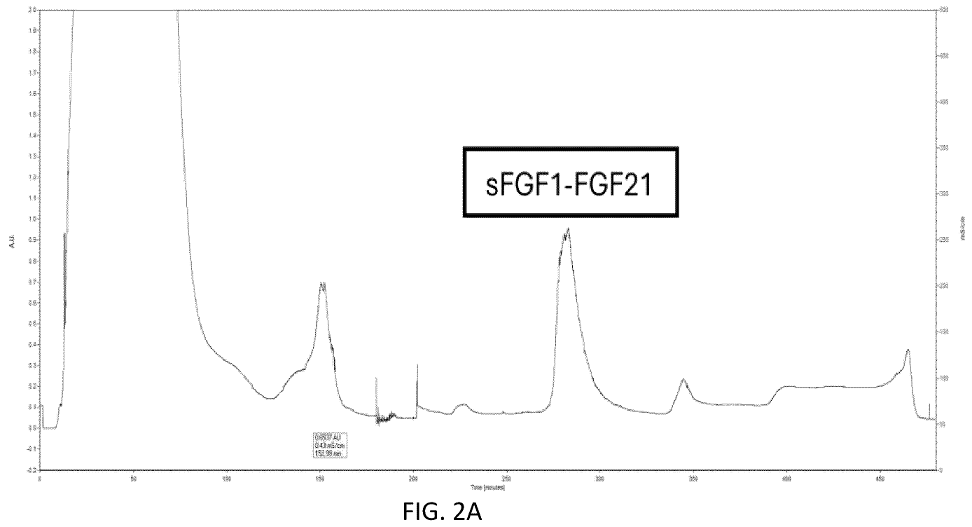

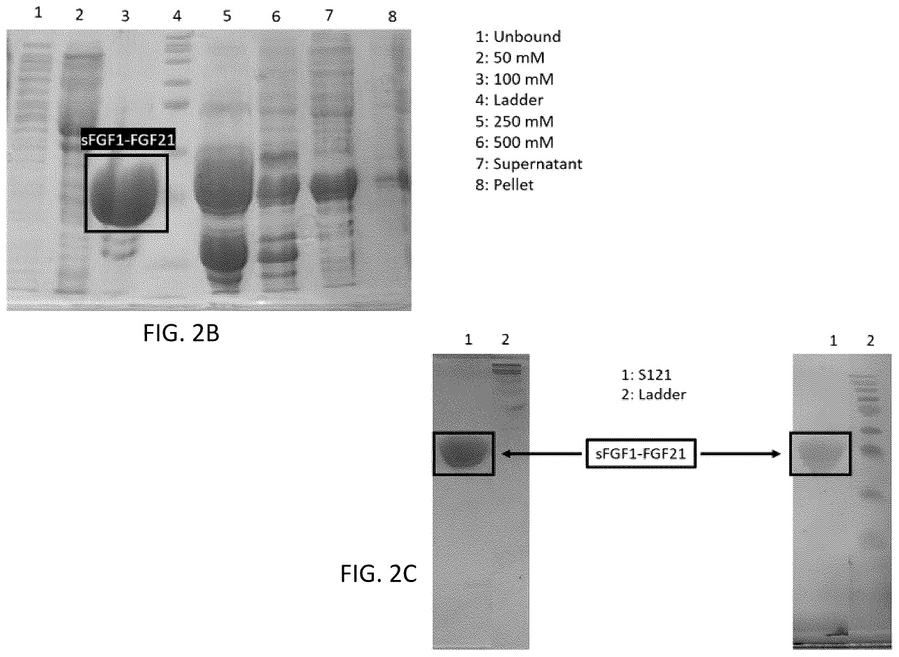

The his-tagged sFGF1-FGF21 was shown to be expressed in BL21 Star (DE3) and purified using Ni²⁺ Sepharose column (FIG. 2A) at around 100 mM imidazole (IMD). After purification, samples were run on SDS-PAGE, and the protein elution was confirmed at 100 mM IMD (FIG. 2B). After concentration and buffer exchange, sFGF1-FGF21 was shown to be pure on SDS-PAGE gel (FIG. 2C) with a concentration of 3.3 mg/mL and a yield of 33 mg/L of cell culture.

In contrast, the wtFGF1 variant, sFGF1, can be easily expressed in BL21 Star (DE3) like wtFGF1 and purified with Heparin Sepharose column due to the protein's poor affinity to heparin. sFGF1 elutes at an unbound fraction (unbound 2), with high purity and concentration on SDS-PAGE gel.

Western blot and mass spectrometry (MS) were used to authenticate the product's identity after Ni²⁺-Sepharose purification. Similar to rFGF21, sFGF1-FGF21 design has a his-tag. Therefore, utilizing anti-his antibody is a reasonable way to verify the product as his-tagged sFGF1-FGF21 on western blot. Indeed, the product from Ni²⁺ Sepharose purification reacted to the antibody and showed a band between 15-30 kDa. Using electrospray ionization (ESI)-MS, the purified product was analyzed to determine its molecular weight as 22.846 kDa. sFGF1-FGF21 was subjected to protein sequencing via trypsin digestion, and fragments were analyzed using matrix-assisted laser desorption-ionization time of flight mass spectrometry (MALDI-TOF-MS). The fragments' amino acid sequences were identified and matched that of sFGF1-FGF21, verifying that the purified product from purification was the designated sFGF1-FGF21.

Combining the DNA sequencing results, protein sequencing results, western blot results, and mass spectrometry results, the purified product after Ni²⁺-Sepharose purification was concluded to be sFGF1-FGF21 using an affordable and easy-to-use model BL21Star (DE3) bacterial line. This results in highly pure products with good yields that can meet experiment demands.

Biophysical Characterizations of sFGF1-FGF21

After purification of the novel sFGF1-FGF21, it is vital to investigate the biophysical characteristics of this new fusion protein in comparison to its wild-type counterparts, wtFGF1 and rFGF21, and sFGF1. Using the characterizations of wtFGF1 and rFGF21, we compared the features of sFGF1-FGF21 and sFGF1 to these proteins to further elucidate the differences between endocrine and paracrine FGFs' structures and features, along with confirming that the addition of FGF21 C-terminus to sFGF1 do not alter the fusion protein's biophysical characteristics and subsequent stability when compared to sFGF1.

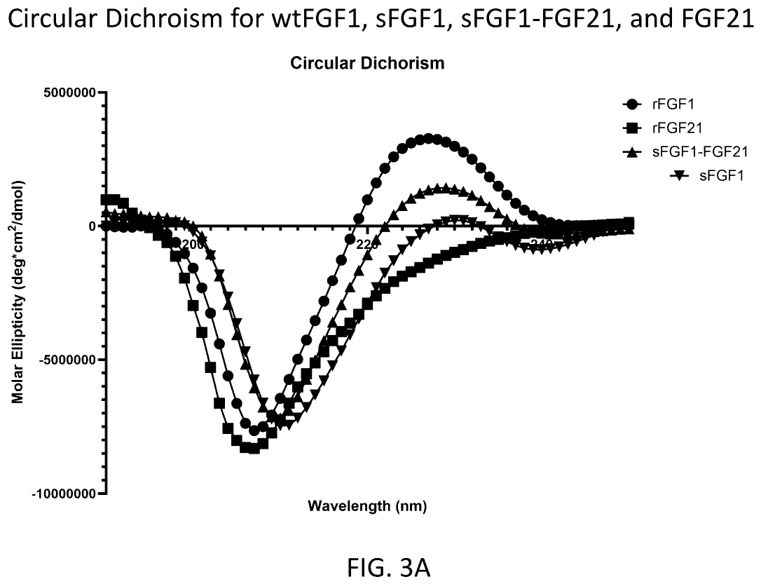

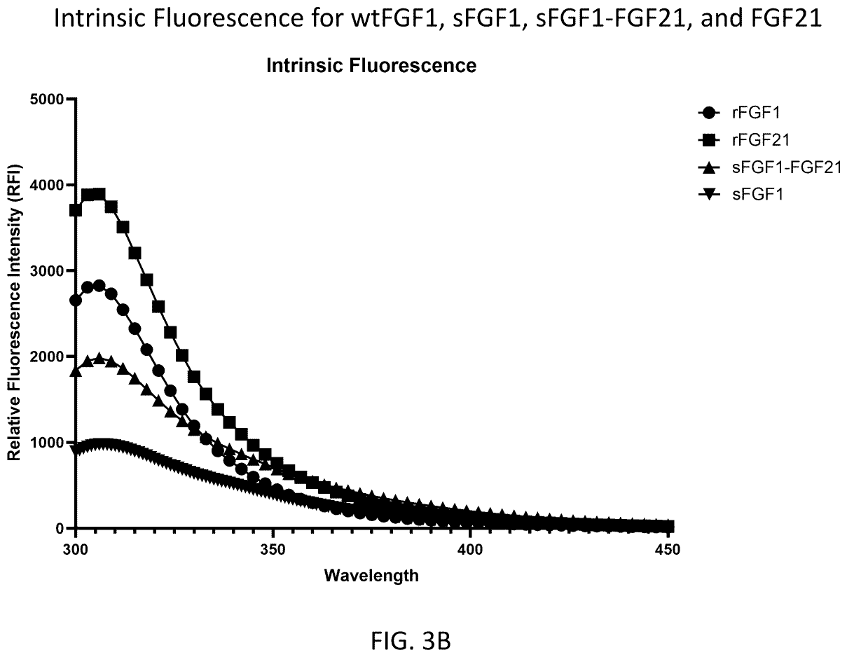

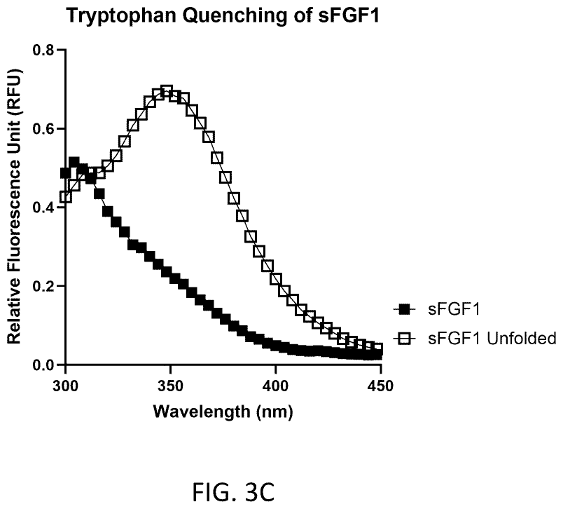

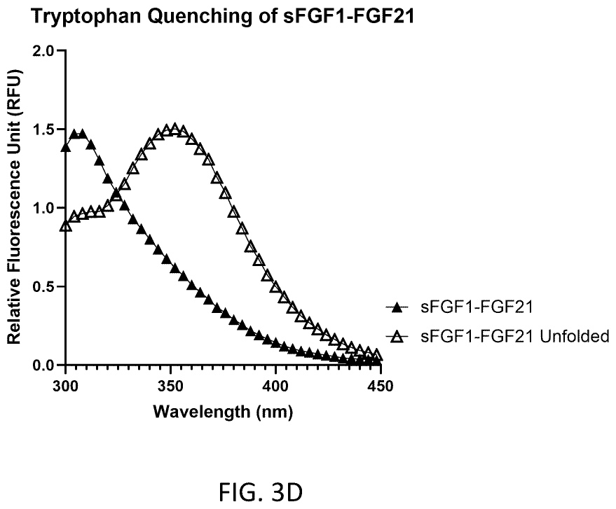

The circular dichroism (CD) far-UV spectrum of the chimeric protein sFGF1-FGF21 has a negative trough around 209 nm that indicates a β-sheet core and a positive peak at 228 nm, which is consistent with features of a β-trefoil core and similar to wtFGF1 and sFGF1 (FIG. 3A). rFGF21, as shown previously, has one negative peak around 207 nm, which is a typical β-sheet structure and may indicate the existence of a β-trefoil core that is conserved in all FGFs [14]. Since the mature FGF21 does not have tryptophan aromatic residue and wtFGF1 has one vital tryptophan residue [38], it may imply that the lack of a positive peak at 228 nm in rFGF21 is due to this absence of tryptophan residue. Compared to the wild-type counterparts, sFGF1-FGF21 seems to follow the typical characteristics of wtFGF1 and its variant, sFGF1, rather than the rFGF21. This result follows our hypothesis as the fusion protein possesses the FGF1-derived core and, thus, should have similar characteristics to wtFGF1 and sFGF1.

Likewise, the intrinsic fluorescence of sFGF1-FGF21 and the counter wild-type, wtFGF1, are similar, with a tyrosine emission peak at 306-309 nm. wtFGF1 and sFGF1 both have only one tryptophan residue and eight tyrosine residues (FIG. 3B). Due to the microenvironment that has nitrogen-containing heterocyclic groups (histidine and proline) surrounding the tryptophan amino acid in both the proteins' conformation, the tryptophan

emission is often quenched [38], as shown in FIG. 3C. Once the protein unfolded, the nitrogen-containing sidechains would move away from the Tryptophan¹²¹ and shift intrinsic spectra from 308 nm to 350 nm as polar solvent-exposed tryptophan emission can now be observed (FIG. 3C). sFGF1-FGF21 also followed this unique feature of wtFGF1 and variants since its intrinsic fluorescence also shifted from 306 (tyrosine emission) to 350 nm (tryptophan emission) as the protein unfolded (FIG. 3D). Thus, like wtFGF1 and sFGF1, the chimeric protein obtained from purification is correctly folded.

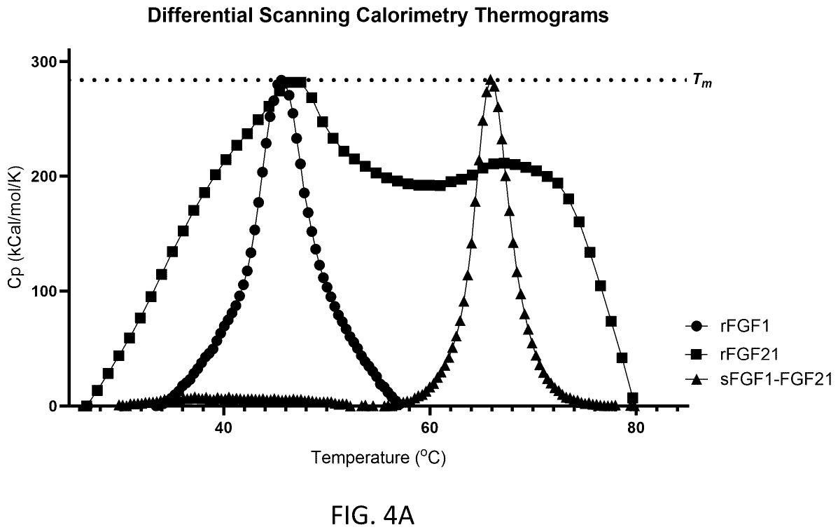

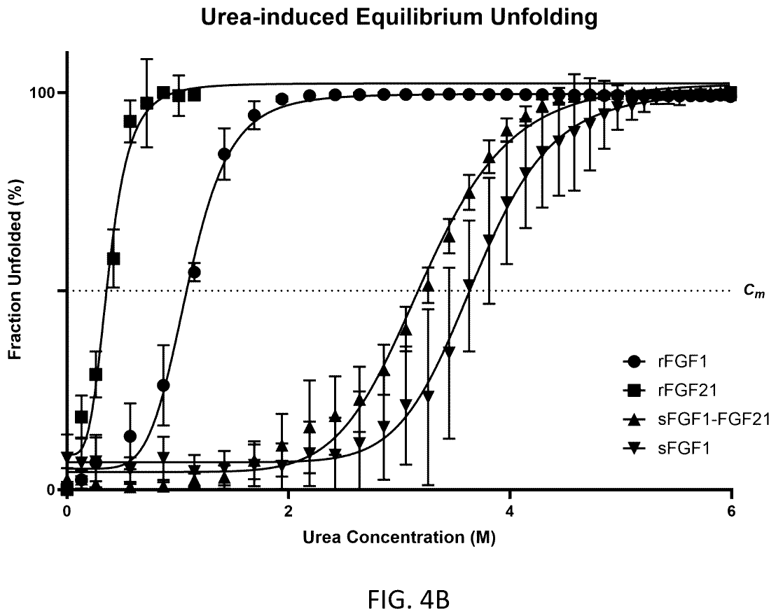

Due to similar biophysical features that sFGF1-FGF21 shared with wtFGF1 and sFGF1, we believe that this fusion protein will also possess similar hyper-stability characteristics from sFGF1. Indeed, sFGF1-FGF21 showed high thermal and chemical stability like sFGF1 (68.03±0.43° C.). Differential scanning calorimetry showed that the Tm of sFGF1-FGF21 is at 65.92° C. (±0.1° C.) (FIG. 4A). The fusion protein is more thermally stable than both wild-type counterparts wtFGF1 (Tm around 45.47±0.14° C.) and rFGF21 (Tm around 46.7±0.1° C.). The results confirmed the design to be highly stable with the change of temperatures, like sFGF1, which corroborates our hypothesis. It should be noted that rFGF21 apparent Tm is at 37° C., lower than the value reported here [13]. rFGF21 is the only protein that has a broad peak and may indicate that the protein goes through multiple thermal-unfolding states [13]. However, the fusion protein does not possess this same characteristic.

Similarly, sFGF1-FGF21 is very resistant to denaturant-induced unfolding (8 M urea) like sFGF1 (Cm of 3.63±0.16 M). The urea-induced unfolding curves (pH=7.2) of sFGF21-FGF21 showed Cm of 3.26±0.04 M, indicating that the fusion protein is more chemically stable than both wild-type counterparts rFGF21 (Cm of 0.42±0.07 M) and wtFGF1 (1.15±0.02 M) (FIG. 4B). Altogether, sFGF1-FGF21 inherits sFGF1's high thermal and chemical stability, overcoming the wild-type rFGF21 instability problem and making this fusion protein a promising therapeutical agent.

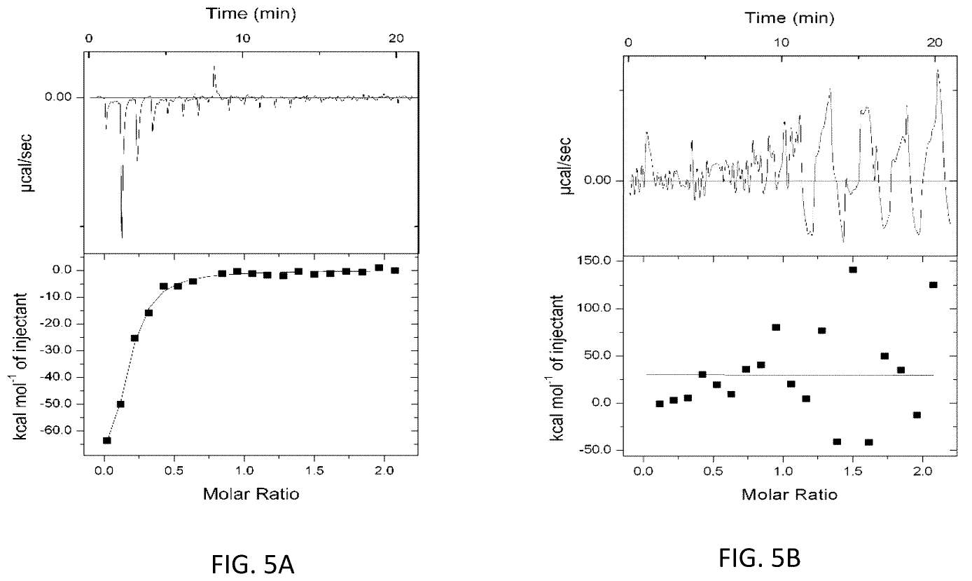

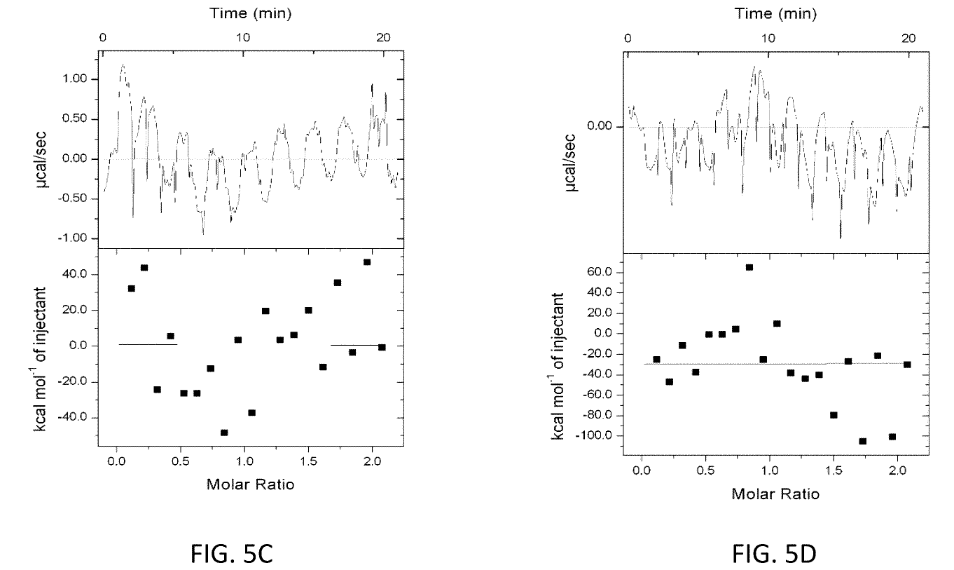

A standard technique to measure the binding affinity of protein-ligand interaction is isothermal titration calorimetry (ITC) [27]. We utilized ITC to measure sFGF1-FGF21's affinity to heparin and compare that to wtFGF1, sFGF1, and rFGF21's affinity to low-molecular-weight HS (~3 kDa). wtFGF1 is the only protein with high affinity to heparin (Kd~2.88±1.32 μM), corroborated by other reports [27, 39] (FIG. 5A). rFGF21 is reported to have a naturally low affinity to heparin [39], and is confirmed by our data (FIG. 5D). sFGF1 was also designed to have low heparin affinity [36], which is also verified in our results (FIG. 5B). Following rFGF21 and sFGF1's behaviors, sFGF1-FGF21 also showed no heparin binding affinity (FIG. 5B) and indicates that sFGF1-FGF21 may act biologically similar to rFGF21 and sFGF1.

Both wtFGF1 and rFGF21 contained a high amount of lysines and arginines. Eleven lysines and six arginines for wtFGF1 [27], ten arginines and four lysines for rFGF21, and both are prone to proteolysis [16, 36, 40]. It is also reported that rFGF21 is specifically prone to proteolysis at the C-terminus portion [16, 40]. With a high number of positively charged residues, we utilized limited trypsin digestion to investigate and compare these proteins' proteolysis tendencies. sFGF1-FGF21 also possesses a high number of charged residues (ten lysines, seven arginines), higher than sFGF1 (ten lysines, five arginines) [36]. Therefore, the protein may be prone to proteolysis like rFGF21. Herein, we examined the sFGF1-FGF21 tendencies to proteolysis and structural flexibility of the protein and compared it to sFGF1 as well as the reported behaviors of wtFGF1, sFGF1, and rFGF21 in trypsin.

In concentration-dependent trypsin assay, sFGF1-FGF21 is shown to have low resistance against proteolysis at low trypsin concentration (0-2.145×10-8 M, equivalent to 0.0005 mg/mL) after 40 minutes at 37° C. At 0.00005 mg/mL trypsin concentration, only ~25% of protein was left undigested. This trypsin concentration was used for a time-dependent trypsin assay from 0-80 minutes and compared to wtFGF1, sFGF1, and rFGF21.

The result showed that sFGF1-FGF21 indeed does not have resistance to trypsin cleavage and is digested rapidly with around 4.24±3.47% of undigested protein after 80 minutes. sFGF1 showed high resistance to trypsin, with 93.20±4.32% of protein remaining undigested. wtFGF1 also is markedly resistant to trypsin digestion than sFGF1-FGF21, with 78.75±5.78% protein remaining undigested at the 80-minutes mark. Even rFGF21, which is prone to proteolysis, showed higher resistance than the fusion protein, with 12.42±4.17% of protein left undigested. The chimeric protein was unable to retain sFGF1 high resistance to proteolysis. This phenomenon may be caused by the FGF21 C-terminus portion of the fusion protein, which is reported to be disordered, long, flexible, and a location for proteolysis [16, 40]. The specific cleaving target is hypothesized to be the Arginine¹⁵⁰ residue located on the FGF21-C-terminus region, as it is a positively charged residue with an accessible location and may be an ideal target for trypsin. The chimera protein was cleaved by trypsin to the sFGF1-like fragment (~15 kDa) of sFGF1-FGF21. This fragment was not further digested into smaller pieces by trypsin at any time during the 80-minute digestion period, indicating that this fragment of sFGF1-FGF21 is very resistant to trypsin and could lend future variants of sFGF1-FGF21 its high proteolysis resistance if Arginine¹⁵⁰ is omitted. Thus, the Arginine¹⁵⁰ residue can be targeted in future mutagenesis work to rectify this undesirable low resistance to proteolysis features of the fusion protein.

Further information about sFGF1-FGF21 tertiary structures was investigated by a binding assay to a non-polar dye called 8-anilinonaphthalene-1-sulfonate (ANS). This extrinsic fluorophore binds to the solvent-exposed hydrophobic pockets to fluoresce within proteins. These pockets are often enclosed within the core of the protein structure. Thus, the relative ANS fluorescence intensity is directly proportional to the amount of solvent-exposed hydrophobic pockets [41]. The results indicate that sFGF1-FGF21's solvent-exposed hydrophobic pockets are similar to that of sFGF1. ANS binding curve of sFGF1 peaks at 140 nM of ANS (89.56±1.03 RFU), and sFGF1-FGF21 is at 130 nM of ANS (82.14±1.88 RFU). Both are much smaller than the wild-type counterparts wtFGF1 and rFGF21. rFGF21's binding curve peaks at 240 nM of ANS (110.4±0.3 RFU) and wtFGF1's maximum fluorescence is at 220 nM ANS (128.73±3.35 RFU). The data showed that sFGF1 and sFGF1-FGF21 have less solvent-exposed hydrophobic pocket regions than the wild-type counterparts. It may also indicate that sFGF1 and sFGF1-FGF21 exhibit increased compactness at their cores and have less core flexibility compared to wtFGF1 and rFGF21, which are more structurally flexible at the core. Therefore, sFGF1-FGF21 is more similar to sFGF1 and possesses less solvent-accessible non-polar pockets, as well as less structurally flexible and more compact at the core than wild-type counterparts, which may contribute to its high thermal and chemical stability.

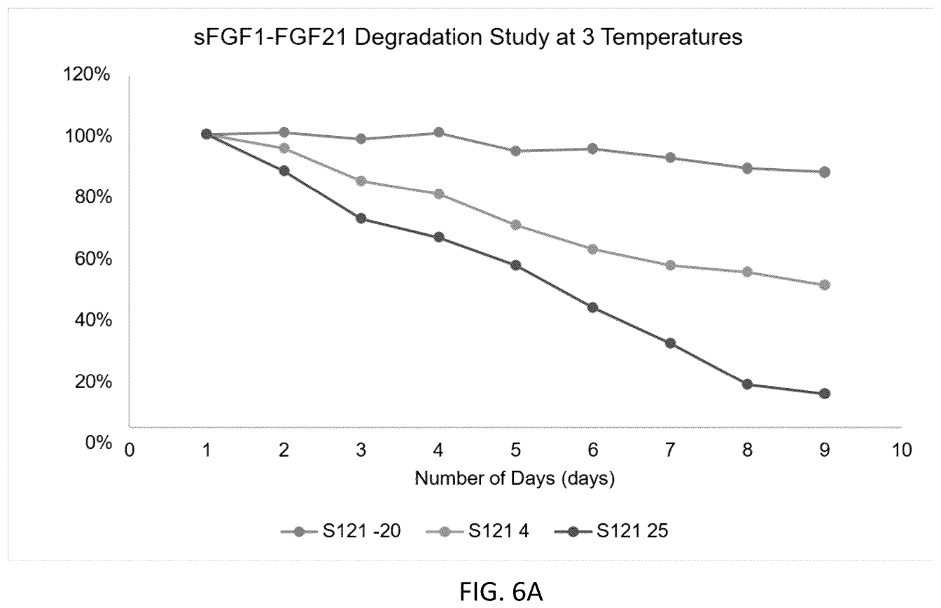

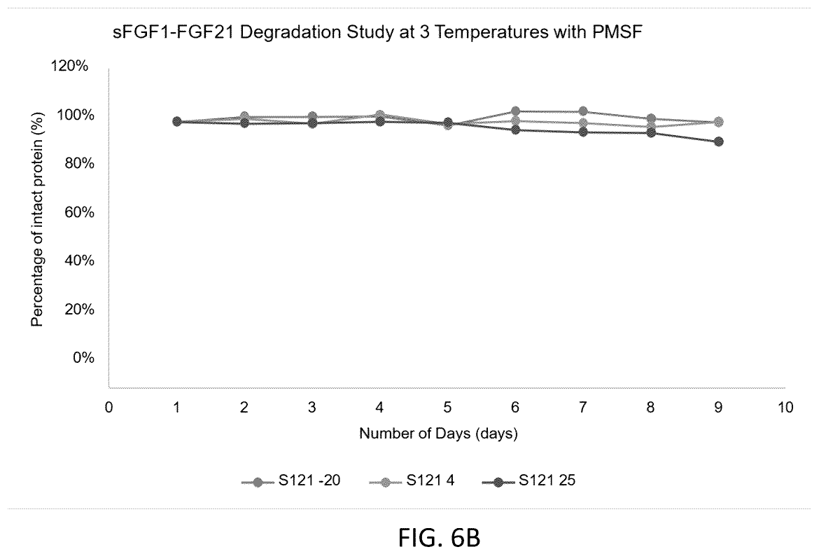

Previously, we showed that rFGF21 is not stable in storage without PMSF. Since sFGF1-FGF21 contains the unstable FGF21 C-terminus, we examined the sFGF1-FGF21 stability over time in different storage conditions. Similar to the rFGF21 experiment, sFGF1-FGF21 was stored in normal working buffer conditions at three temperatures (−20° C., 4° C., 25° C.) and monitored over nine days, and ran on SDS-PAGE. Gels were quantified using densitometric scan software (UN-SCAN-IT, Silk Scientific, USA), and the percentage of intact proteins was calculated and graphed. Similar to rFGF21, sFGF1-FGF21 showed rapid degradation at 25° C., slow degradation at 4° C., and remained mostly intact at −20° C. (FIG. 6A). The fusion protein was seen to degrade from the C-terminus and into the sFGF1 fragment, where no further degradation occurs, as shown in immunoblotting result using anti-his antibody. The blot showed that the his-tags, located at the N-terminus, are still attached to the degraded proteins, indicating that the degradation happens at the C-terminus.

sFGF1-FGF21 was also added to a new buffer condition with one mM PMSF and monitored over nine days, the same way as the previous experiment. The new condition showed sFGF1-FGF21 with almost no degradation over nine days in all storage temperatures (FIG. 6B), similar to rFGF21. This observation indicates that sFGF1-FGF21 is unstable and degraded quickly without PMSF at room temperature or in cold storage. This could be due to the flexibility of the FGF21 C-terminus, enhanced by multiple flexible glycine residues in the regions.

To investigate where the sFGF1-FGF21 is degraded, the degraded fragments of liquid and in-gel samples of sFGF1-FGF21 after nine days were subjected to ESI-MS to analyze and identify the fragments' molecular weights and sequences. The result showed that sFGF1-FGF21 degraded into smaller fragments, with most starting at Arginine¹⁵⁰ residue and bigger fragments containing the sFGF1 portion. Combining with the previous limited trypsin digestion results, the stability over time data, and the MS data, one can observe that sFGF1-FGF21 degrades at the C-terminus, specifically at the position Arginine¹⁵⁰ where the protein is cleaved off, leaving a bigger fragment containing the sFGF1 portion of the fusion protein and a smaller FGF21 C-terminus fragment.

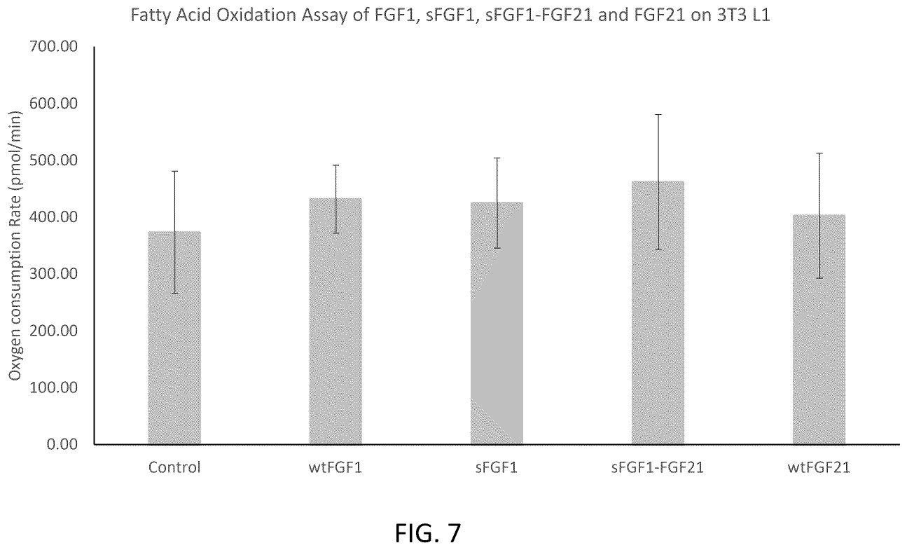

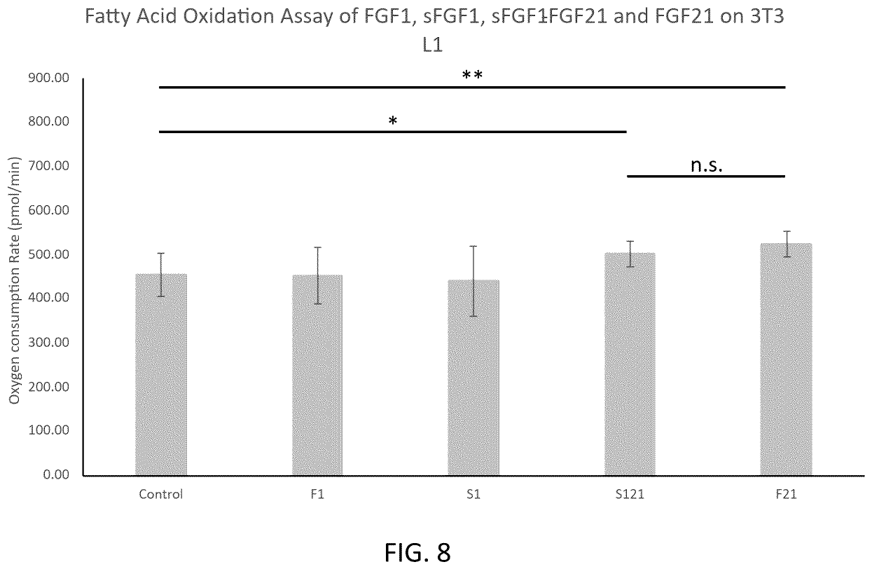

To examine sFGF1-FGF21's ability to increase fatty acid oxidation, 3T3 L1 adipocytes were utilized. Similar to the other metabolic assay described previously, pre-adipocytes were grown to 70% confluence, differentiated to become mature adipocytes through chemical differentiation process, and incubated with proteins and a source of fatty acids for 12 hours before their oxygen consumption rate and pH changes were measured using a Seahorse XF Pro analyzer. At the lower concentration of 0.625 nM, sFGF1-FGF21 showed higher endogenous fatty oxidation than controls and FGF21 (FIG. 7). Moreover, when the exogenous fatty acid (palmitate) was added, sFGF1-FGF21 also showed an increase in oxidation rate relative to controls (FIG. 8). These activities could be due to metabolic activities of the FGF21 C-terminus portion of the fusion protein, which is involved in increasing fatty acid oxidation, as described above. These results indicate that the chimeric protein retains the fatty acid oxidation enhancing activity of FGF21 and that it may be more metabolically active than the wild-type protein in oxidizing endogenous fat.

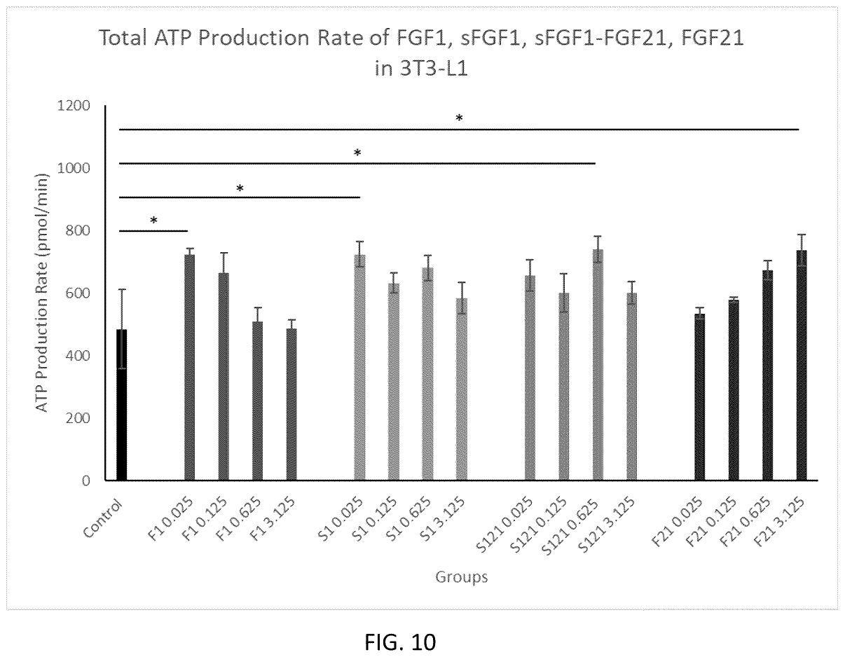

To examine the ability of sFGF1-FGF21 to enhance ATP production, 3T3 L1 adipocytes were utilized. Cells were grown to 70% confluence, differentiated to become mature adipocytes through chemical differentiation process, and incubated with proteins for 12 hours before their oxygen consumption rate and pH changes were measured with a Seahorse XF Pro analyzer. sFGF1-FGF21 showed an increased ATP production rate, especially at lower concentration of 0.625 nM, when compared to control and other protein groups (FIG. 10). These activities could be due to metabolic activities of the FGF21 C-terminus, which can interact with receptors in adipocytes to increase uptake glucose from glucose-rich media, allowing more glucose to be oxidized in mitochondria, and more ATP to be produced. This result indicates that the chimeric protein retains the biological activities of FGF21 and is more metabolically active than FGF21 in oxidizing glucose.

### CONCLUSIONS

The novel design of sFGF1-FGF21 was successfully produced and verified using a bacterial expression system. This fusion protein possesses many biophysical features that are shared with sFGF1, including low HS binding, a canonical β-trefoil core, and a tyrosine emission intrinsic fluorescence with quenched tryptophan emission. The novel chimeric protein also retains many beneficial sFGF1 features like high thermal and chemical resistance and increased core compactness, which overcomes rFGF21's original challenges with its low stability. However, sFGF1-FGF21 still faced similar rFGF21 problems at its flexible C-terminus, which is low resistance to proteolysis and prone to degradation, possibly at the Arginine¹⁵⁰ residue in the C-terminus. In conclusion, this aim was achieved through the production of the novel sFGF1-FGF21 that retains some beneficial sFGF1's hyper-stable characteristics.

### Materials

The competent cells were XL10 gold (Agilent Technologies, USA), Rosetta-gami (Sigma and MP Biomedicals, LLC.), and BL21 Star DE3 (Thermo Fisher Scientific, USA). IBI Scientific (USA) supplied the Luria-Bertani (LB) broth. Qiagen Inc. (USA) supplied the plasmid isolation kit. GE Healthcare (USA) supplied both the Heparin Sepharose and Nickel Sepharose resins. VWR Scientific (USA) provided the buffer components (tris, NaCl, glycine, Na2HPO4, NaH2PO4), acrylamide, bis-acrylamide, Coomassie blue, ethanol, methanol, urea crystal, acetic acid, Tween 20, glycerol. Phenylmethylsulfonyl fluoride (PMSF), low molecular weight (~3000 Da) heparin sodium salt, and trypsin (from bovine pancreas) were provided by Sigma and MP Biomedicals (USA). High molecular-weight heparin is polydisperse by nature, which prompted us to use low molecular-weight heparin instead. Ammonium Sulfate was from EMD. Alkaline phosphatase anti-His antibody [HIS-1] is from Abcam, UK. Nitrocellulose membrane 0.2 μm was from Bio-Rad Laboratories, Inc. (USA). Low molecular weight (~3000 Da) heparin sodium salt was obtained from Sigma and MP Biomedicals (USA). All other chemicals and materials were of high-quality analytical grade, as described elsewhere [42]. Unless otherwise stated, samples were made in the working buffer of 10 mM phosphate buffer, 150 mM NaCl, and 25 mM (NH4) 2SO4 at pH 7.2.

Construction and Purification of sFGF1 and sFGF1-FGF21

The super acidic human FGF1 (sFGF1) sequence, plasmid, and glycerol were obtained through the Kumar's Lab [43]. Design of super acidic human FGF1 fused with FGF21 C-terminal (residues 161-209) (sFGF1-FGF21) was cloned (Genscript, USA) into the pET21a+ vectors with ampicillin-resistant gene. Both sFGF1 and sFGF1-FGF21 were transformed into highly competent bacterial cells XL 10 Gold to produce more plasmids as recommended by the manufacturer. Plasmid isolation of sFGF1 and sFGF1-FGF21-containing XL 10 Gold cells was conducted following the manufacturer's recommendation. The resulting plasmids were run on 1% Agarose Gel Electrophoresis (AGE) to determine their existence and purity. The plasmid concentrations were determined via Nanodrop Microvolume Spectrophotometers (Thermo Fisher Scientific, USA) within the double-stranded DNA concentration estimation protocol. Plasmid was sent for DNA sequencing to confirm identity (Plasmidsaurus, USA).

BL21 Star (DE3) was used to express both proteins. Both were grown in LB media at 37° C., 240 rpm to an Optical Density of 0.6-8.0 at 600 nm absorbance wavelength and incubated with one mM IPTG for 5 hours at 37° C., 240 rpm. Cells were centrifuged to collect as pellets and resuspended in the working buffer. Afterward, induced cells were lysed on ice by Sonifier SFX150 (Branson Ultrasonics, USA) for 75 cycles with 10-second on/off pulses for a total of 30 minutes with a 15-minute break in between to let the solution cool down. The supernatant was isolated from the cell debris at 19,000 rpm using centrifugation by Avanti J-25 Centrifuge (Beckman Coulter, USA) for 25 minutes. The supernatant of sFGF1-FGF21 was run over the Nickel Sepharose affinity column that was pre-equilibrated with a working buffer at 1 millimeter per minute. A stepwise imidazole (IMD) gradient (20-500 mM IMD) was used to purify and elute the product. The supernatant of sFGF1 was run on a pre-equilibrated Heparin Sepharose column with the working buffer. The protein was eluted through a stepwise NaCl gradient from 100 mM to 1500 mM, as described elsewhere [42]. The purification process was monitored at 280 nm wavelength. The products' purity was verified using SDS-PAGE (15%) at 200 V for 50 minutes, stained with Coomassie brilliant blue. The Bradford method and Nanodrop Microvolume Spectrophotometers (Thermo Fisher Scientific, USA) (using the 280 nm estimation protocol) were used to determine the concentration of the products [44, 45].

Predictor of Natural Disordered Regions (PONDR) Analysis of sFGF1-FGF21

PONDR analysis was performed using the tools available at //www.pondr.com/. After the sFGF1-FGF21 sequence was input into the appropriate space, predictor VLXT was chosen. The chosen output options were graphics, statistics, and sequence reports. The result was collected and displayed as is.

Structure Prediction of sFGF1-FGF21 Using ColabFold (AlphaFold 2)

sFGF1-FGF21 prediction was also made using the AlphaFold 2/ColabFold prediction program. Within the ColabFold notebook v1.5.2 (available at //github.com/sokrypton/ColabFold), AlphaFold2_mmseqs2 was chosen. The chimeric protein sequence was input into the notebook. Other options that were chosen: the database that was searched against (msa_mode) is UniRef and Environmental (mmseqs2_uniref_env); the number of models unused (num_relax) is 0. After connecting to an available machine in AlphaFold 2, the setting was run, and a structure of the sFGF1-FGF21 was produced by choosing the best model out of all five trained models' predictions in the server. Blue-colored regions are low in error estimate, red is medium, and yellow is high according to the pLDDT score.

### Western Blot

Since the sFGF1-FGF21 design has a Histidine tag at the N-terminus, a western blot was carried out to confirm the identity of the product using alkaline phosphatase anti-6× his antibody. The purified product that was thought to be sFGF1-FGF21, at concentration of 31.4 μM, was run on 15% SDS-PAGE gel for 50 minutes at 200 V. This gel was subjected to the transfer process to transfer the protein sample from gel onto the nitrocellulose membrane 0.2 μm through electrophoresis in Towbin (25 mm Tris, 192 mM glycine, pH 8.3, 20% v/v methanol). The current was set at 150V and current at 80 mA for 120 minutes. The membrane, now with the transferred protein, was soaked in 5% skim milk solutions in TBS-T solution (20 mM Tris, 150 mM NaCl, 0.1% w/v Tween 20) for 30 minutes and rinsed with TBS-T. The membrane was subjected to 0.2% BSA in TBS-T with added anti-6× antibody and incubated at room temp overnight. NBT/BCIP solution was added the following day to visualize the bands on the membrane, which represented the His tag-containing product, confirming the identity of the product as sFGF1-FGF21.

### Mass Spectrometry

A liquid sample of sFGF1-FGF21, with concentrations of 31.4 μM, was subjected to liquid chromatography and electro-spray ionization-mass spectrometry. Each sample's molecular weight was determined. sFGF1-FGF21 were also undergoing the protein sequencing process using MS to confirm their identities. Protein samples were analyzed by in-gel trypsin digestion and/or in-solution trypsin digestion. The fragments were analyzed using MALDI-TOF-MS to confirm the fragments' sequences and identities, proving the existence of sFGF1-FGF21 identities in products.

### CD and Fluorescence Spectroscopy

J-1500 Spectrophotometer (JASCO Inc., Japan) with circular dichroism detection provided the CD spectra for sFGF1-FGF21 and sFGF1 to determine sFGF1 and sFGF1-FGF21's secondary features. For analysis, 31.4 μM of protein was added into a 0.1 cm path-length quartz cell with 10 mM PB and 100 mM NaCl. The wavelength was set to 190-250 nm at room temperature, 20 nm/min scanning speed. This procedure was repeated for ten scans, and the average data was collected. The final CD data was converted to molar ellipticity.

The tertiary structure of sFGF1-FGF21 and sFGF1 were studied through intrinsic fluorescence spectroscopy by using the Fluorescence Spectrophotometer F-2500 (Hitachi High-Tech, USA). Specifically, the investigation focused on changes in the emission of tryptophan and tyrosine residues, which fluoresce at 340 and 308 nm. Proteins, concentrations of 31.4 μM, were added to the working buffer. The excitation wavelength was set at 280 nm, and the emissions were from 300-450 nm. A buffer scan was done and subtracted out of the spectra to correct background noise.

### 8-Anilino-1-Napthalenesulfonic Acid (ANS) Binding Assay

The ANS binding experiment was done on a fluorescence spectrophotometer F-2500 (Hitachi High-Tech, USA). The excitation wavelength was set at 380 nm, the emission range at 450-600 nm, 2.5 nm slit width. Protein samples (31.4 μM) were placed in a 10 mm quartz cuvette in the working buffer. 20 mM of ANS stock was titrated into the protein mixture in 10 μM increments, mixed, and incubated at room temperature for 2 minutes before each reading. Fluorescence intensity was monitored at 520 nm and collected.

### Limited Trypsin Digestion Assay

The limited trypsin digestion of sFGF1 and sFGF1-FGF21 was performed in the working buffer. Eight tubes (tubes 2-9) contained 31.4 μM of protein, and 0.00005 mg/mL concentration of bovine trypsin were mixed to a total volume of 200 μL. The remaining two tubes (tubes 1 and 10, 200 μL each) contained only protein (31.4 μM), and only trypsin of 0.00005 mg/mL was controlled. The experimental tubes were incubated at 37° C. All tubes have a total volume of 200 μL. Different incubation time intervals were set for each tube from 2-9:10 minutes, 20 minutes, 30 minutes, 40 minutes, 50 minutes, 60 minutes, 70 minutes, and 80 minutes.

Samples were removed at specific intervals, as noted above, and 10% trichloroacetic acid was added to terminate the reaction. SDS-PAGE gel (50 minutes at 200 V, 15%) was utilized to analyze the digested samples, stained with Coomassie dye. The percentage of undigested protein was calculated by comparing the band intensity of each sample to the control tube one on the gel using UN-ScanIT densitometric software (Silk Scientific Inc., USA). This percentage was plotted against incubation time intervals. The processes were done in triplicate with three separate assays.

### Differential Scanning Calorimetry (DSC)

The differential scanning microcalorimeter MicroCal PEAQ-DSC (Malvern Panalytical Inc., Netherlands) was used to study the thermal stability of products. The setting temperature range is from 25-80° C. with 1° C. ramping temperatures. sFGF1 and sFGF1-FGF21 were in the concentration of 31.4 μM in the working buffer. Three buffer runs were conducted before the sample run and were subtracted from the sample runs to eliminate noise. Specific heat capacity for each protein was obtained using MicroCal PEAQ-DSC software (21 CFR part 11) and plotted against the temperatures of the run. The thermal transition midpoint (melting temperature Tm) for each sample was calculated and recorded.

### Isothermal Titration Calorimetry (ITC)

The MicroCal iTC200 microcalorimeter (Malvern Panalytical Inc., Netherlands) was utilized to investigate sFGF1-FGF21 and sFGF1's binding affinities to cofactor heparin. To keep the protein: ligand ratio to 1:8, the degassed protein concentration was set at 31.4 μM, and the heparin concentration (in the syringe, degassed) was at 251.2 μM). The heparin injection volume was 2 μL for a total of 20 titrations and was conducted at 25° C. The data was analyzed through the accompanied software Origin (Malvern Panalytical Inc., Netherlands).

### Equilibrium Unfolding Using Chemical Denaturant.

To study the effect of chemical denaturants such as urea on sFGF1-FGF21 and sFGF1, a J-1500 Spectrophotometer (JASCO Inc., Japan) was used. Proteins with a concentration of 31.4 μM in the working buffer were titrated with 8 M urea stock solution. This denaturation process was tracked using the spectrophotometer's automatic scanning titrations with fluorescence excitation/emission mode. The excitation wavelength was set at 280 nm; emission wavelengths were set from 300-450 nm. The fluorescence measurements under increasing urea concentrations from 0-6 M were recorded, and a ratio between 305 and 350 nm peaks was calculated for each urea concentration. The fraction of protein unfolded in percentage was calculated for each urea concentration and plotted for each protein as reported elsewhere [46]. The chemical transition midpoint (Cm) for each sample was recorded.

Stability of rFGF21 in Storage Over Time

To investigate sFGF1-FGF21 stability in storage, we designed an experiment in which three different storage conditions were set up, with three temperatures: −20° C., 4° C., and 25° C., and the protein was stored in these conditions over nine days. Samples of the protein, at each temperature, were taken every day for nine days. Samples were analyzed on 15% SDS-PAGE and were digitized through a densitometric scanning software UN-SCAN-IT (Silk Scientific, USA) for all nine days. Degraded and intact protein levels for each day were calculated based on a comparison with protein on the first day in percentage and plotted as days versus percentage of intact protein. Degraded samples on the gel after nine days were sent to mass spec to investigate their molecular weights and protein sequences to identify the point of degradation/proteolysis of sFGF1-FGF21. The liquid-degraded sample of sFGF1-FGF21 was subjected to ESI-MS to confirm the degraded sample's molecular weight. Liquid and in-gel samples of degraded sFGF1-FGF21 were subjected to MALDI-TOF-MS as described above to identify the sequence of the degraded protein. Comparing both the molecular weight and protein sequences of the degraded sample to intact sFGF1-FGF21, one can identify possible residue(s) that were cleaved by proteolysis/degraded over time.

sFGF1-FGF21 was subjected to a second storage variable by adding one mM phenylmethylsulphonyl fluoride (PMSF) to the buffer. This sample underwent the same protocol as described above, and a new plot was made to reflect the effects of PMSF on sFGF1-FGF21 stability over time.

### REFERENCES

1. Chen Z, Yang L, Liu Y, Huang P, Song H, Zheng P. The potential function and clinical application of FGF21 in metabolic diseases. Frontiers in pharmacology. 2022; 13:1089214. doi: 10.3389/fphar.2022.1089214.
2. Li X. The FGF metabolic axis. Frontiers of medicine. 2019; 13 (5): 511-30. doi: 10.1007/s11684-019-0711-y.
3. Hui Q, Jin Z, Li X, Liu C, Wang X. FGF Family: From Drug Development to Clinical Application. International journal of molecular sciences. 2018; 19 (7): 1875. doi: 10.3390/ijms19071875.
4. Kuro-o M. Endocrine FGFs and Klothos: emerging concepts. Trends in endocrinology and metabolism: TEM. 2008; 19 (7): 239-45. doi: 10.1016/j. tem.2008.06.002.
5. Schlessinger J, Plotnikov A N, Ibrahimi O A, Eliseenkova A V, Yeh B K, Yayon A, et al. Crystal structure of a ternary FGF-FGFR-heparin complex reveals a dual role for heparin in FGFR binding and dimerization. Molecular cell. 2000; 6 (3): 743-50. doi: 10.1016/s1097-2765 (00) 00073-3.
6. Yie J, Wang W, Deng L, Tam L T, Stevens J, Chen M M, et al. Understanding the physical interactions in the FGF21/FGFR/β-Klotho complex: structural requirements and implications in FGF21 signaling. Chemical biology & drug design. 2012; 79 (4): 398-410. doi: 10.1111/j.1747-0285.2012.01325.x.
7. Owen B M, Ding X S, Morgan D A, Coate K C, Bookout A L, Rahmouni K, et al. FGF21 Acts Centrally to Induce Sympathetic Nerve Activity, Energy Expenditure, and Weight Loss. Cell Metabolism. 2014; 20 (4): 670-7. doi: 10.1016/j.cmet.2014.07.012.
8. Degirolamo C, Sabbà C, Moschetta A. Therapeutic potential of the endocrine fibroblast growth factors FGF19, FGF21 and FGF23. Nature Reviews Drug Discovery. 2015; 15 (1): 51-69. doi: 10.1038/nrd.2015.9.
9. Falamarzi K, Malekpour M, Tafti M F, Azarpira N, Behboodi M, Zarei M. The role of FGF21 and its analogs on liver associated diseases. Frontiers in medicine. 2022; 9:967375. doi: 10.3389/fmed.2022.967375.
10. Geng L L, Lam K S L, Xu A M. The therapeutic potential of FGF21 in metabolic diseases: from bench to clinic. Nature Reviews Endocrinology. 2020; 16 (11): 654-67. doi: 10.1038/s41574-020-0386-0.
11. Gaich G, Chien J Y, Fu H, Glass L C, Deeg M A, Holland W L, et al. The effects of LY2405319, an FGF21 analog, in obese human subjects with type 2 diabetes. Cell metabolism. 2013; 18 (3): 333-40. doi: 10.1016/j.cmet.2013.08.005.
12. Adams A C, Halstead C A, Hansen B C, Irizarry A R, Martin J A, Myers S R, et al. LY2405319, an Engineered FGF21 Variant, Improves the Metabolic Status of Diabetic Monkeys. PloS one. 2013; 8 (6): e65763. doi: 10.1371/journal.pone.0065763.
13. Kharitonenkov A, Beals J M, Micanovic R, Strifler B A, Rathnachalam R, Wroblewski V J, et al. Rational design of a fibroblast growth factor 21-based clinical candidate, LY2405319. PloS one. 2013; 8 (3): e58575. doi: 10.1371/journal.pone.0058575.
14. Zhu L, Zhao H, Liu J, Cai H, Wu B, Liu Z, et al. Dynamic folding modulation generates FGF21 variant against diabetes. EMBO reports. 2021; 22 (1): e51352. doi: 10.15252/embr.202051352.
15. Phan P, Saikia B B, Sonnaila S, Agrawal S, Alraawi Z, Kumar T K S, et al. The Saga of Endocrine FGFs. Cells. 2021; 10 (9): 2418. doi: 10.3390/cells10092418.
16. Dunshee D R, Bainbridge T W, Kljavin N M, Zavala-Solorio J, Schroeder A C, Chan R, et al. Fibroblast Activation Protein Cleaves and Inactivates Fibroblast Growth Factor 21. The Journal of biological chemistry. 2016; 291 (11): 5986-96. doi: 10.1074/jbc.M115.710582.
17. R H, Y S L, J S, E B, M H, T H, et al. Rationale-Based Engineering of a Potent Long-Acting FGF21 Analog for the Treatment of Type 2 Diabetes. PloS one. 2012; 7 (11). doi: 10.1371/journal.pone.0049345.
18. Zhen E Y, Jin Z, Ackermann B L, Thomas M K, Gutierrez J A. Circulating FGF21 proteolytic processing mediated by fibroblast activation protein. The Biochemical journal. 2016; 473 (5): 605-14. doi: 10.1042/BJ20151085.
19. Lee K N, Jackson K W, Terzyan S, Christiansen V J, McKee P A. Using substrate specificity of antiplasmin-cleaving enzyme for fibroblast activation protein inhibitor design. Biochemistry. 2009; 48 (23): 5149-58. doi: 10.1021/bi900257m.
20. Edosada C Y, Quan C, Tran T, Pham V, Wiesmann C, Fairbrother W, et al. Peptide substrate profiling defines fibroblast activation protein as an endopeptidase of strict Gly (2)-Pro(1)-cleaving specificity. FEBS letters. 2006; 580 (6): 1581-6. doi: 10.1016/j.febslet.2006.01.087.
21. Dong J Q, Rossulek M, Somayaji V R, Baltrukonis D, Liang Y, Hudson K, et al. Pharmacokinetics and pharmacodynamics of PF-05231023, a novel long-acting FGF21 mimetic, in a first-in-human study. British journal of clinical pharmacology. 2015; 80(5):1051-63. doi: 10.1111/bcp.12676.
22. Zhao L, Niu J, Lin H, Zhao J, Liu Y, Song Z, et al. Paracrine-endocrine FGF chimeras as potent therapeutics for metabolic diseases. EBioMedicine. 2019; 48:462-77. doi: 10.1016/j.ebiom.2019.09.052.
23. Min X, Weiszmann J, Johnstone S, Wang W, Yu X, Romanow W, et al. Agonistic β-Klotho antibody mimics fibroblast growth factor 21 (FGF21) functions. The Journal of biological chemistry. 2018; 293 (38): 14678-88. doi: 10.1074/jbc.RA118.004343.
24. Sonoda J, Chen M Z, Baruch A. FGF21-receptor agonists: an emerging therapeutic class for obesity-related diseases. Hormone molecular biology and clinical investigation. 2017; 30(2). doi: 10.1515/hmbci-2017-0002.
25. Kharitonenkov A, DiMarchi R. FGF21 Revolutions: Recent Advances Illuminating FGF21 Biology and Medicinal Properties. Trends in endocrinology and metabolism: TEM. 2015; 26(11):608-17. doi: 10.1016/j. tem.2015.09.007.
26. Huang J, Ishino T, Chen G, Rolzin P, Osothprarop T F, Retting K, et al. Development of a novel long-acting antidiabetic FGF21 mimetic by targeted conjugation to a scaffold antibody. The Journal of pharmacology and experimental therapeutics. 2013; 346(2):270-80. doi: 10.1124/jpet. 113.204420.
27. Kerr R, Agrawal S, Maity S, Koppolu B, Jayanthi S, Suresh Kumar G, et al. Design of a thrombin resistant human acidic fibroblast growth factor (hFGF1) variant that exhibits enhanced cell proliferation activity. Biochemical and biophysical research communications. 2019; 518(2):191-6. doi: 10.1016/j.bbrc.2019.08.029.
28. Ornitz D M, Itoh N. Fibroblast growth factors. Genome biology. 2001; 2 (3): reviews3005.1. doi: 10.1186/gb-2001-2-3-reviews3005.
29. Brych S R, Blaber S I, Logan T M, Blaber M. Structure and stability effects of mutations designed to increase the primary sequence symmetry within the core region of a beta-trefoil. Protein science: a publication of the Protein Society. 2001; 10(12):2587-99. doi: 10.1110/ps.ps.34701.
30. Zakrzewska M, Krowarsch D, Wiedlocha A, Otlewski J. Design of fully active FGF-1 variants with increased stability. Protein engineering, design & selection: PEDS. 2004; 17(8):603-11. doi: 10.1093/protein/gzh076.
31. Ogura K, Nagata K, Hatanaka H, Habuchi H, Kimata K, Tate S, et al. Solution structure of human acidic fibroblast growth factor and interaction with heparin-derived hexasaccharide. Journal of biomolecular NMR. 1999; 13(1):11-24. doi: 10.1023/a: 1008330622467.
32. Zakrzewska M, Marcinkowska E, Wiedlocha A. FGF-1: from biology through engineering to potential medical applications. Critical reviews in clinical laboratory sciences. 2008; 45(1):91-135. doi: 10.1080/10408360701713120.
33. Agrawal S. Design and characterization of FGF-1 mutant(s) with increased stability and enhanced bioactivity. Chemistry and Biochemistry: University of Arkansas; 2020. p. 52.
34. López-Cano A, Martínez-Miguel M, Guasch J, Ratera I, Arís A, Garcia-Fruitós E.

Exploring the impact of the recombinant Escherichia coli strain on defensins antimicrobial activity: BL21 versus Origami strain. Microbial cell factories. 2022; 21(1):77. doi: 10.1186/s12934-022-01803-7.
35. Mirdita M, Schütze K, Moriwaki Y, Heo L, Ovchinnikov S, Steinegger M. ColabFold: making protein folding accessible to all. Nature Methods. 2022; 19(6):679-82. doi: doi: 10.1038/s41592-022-01488-1.
36. Agrawal S. Design and characterization of FGF-1 mutant(s) with increased stability and enhanced bioactivity. Chemistry and Biochemistry: University of Arkansas; 2020. p. 52.
37. Berkmen M. Production of disulfide-bonded proteins in <i>Escherichia coli</i>. Protein Expression and Purification. 2012; 82(1):240-51. doi: 10.1016/j.pep.2011.10.009.
38. Agrawal S, Govind Kumar V, Gundampati R K, Moradi M, Kumar T K S. Characterization of the structural forces governing the reversibility of the thermal unfolding of the human acidic fibroblast growth factor. Scientific reports. 2021; 11 (1): 15579. doi: 10.1038/s41598-021-95050-2.
39. Kharitonenkov A, Shiyanova T L, Koester A, Ford A M, Micanovic R, Galbreath E J, et al. FGF-21 as a novel metabolic regulator. The Journal of clinical investigation. 2005; 115(6):1627-35. doi: 10.1172/JCI23606.
40. Gillum M P, Potthoff M J. FAP finds FGF21 easy to digest. The Biochemical journal. 2016; 473 (9). doi: 10.1042/BCJ20160004.
41. Davis J E, Alghanmi A, Gundampati R K, Jayanthi S, Fields E, Armstrong M, et al. Probing the role of proline-135 on the structure, stability, and cell proliferation activity of human acidic fibroblast growth factor. Archives of Biochemistry and Biophysics. 2018; 654:115-25. doi: 10.1016/j.abb.2018.07.017.
42. Phan P, Sonnaila, S., Ternier, G., Edirisinghe, O., Okoto, P. S., Kumar, T. K. S. Overexpression and Purification of Mitogenic and Metabolic Fibroblast Growth Factors. Methods in molecular biology (Clifton, NJ). 2024; 2762:151-81. doi: 10.1007/978-1-0716-3666-4_10.
43. Thallapuranam S K, Agarwal S, Gundampati R K, Jayanthi S, Wang T, Jones J, et al. Engineered FGF1 and FGF2 compositions and methods of use thereof. Google Patents; 2022.
44. Hung K W, Kumar, T. K. S., Kathir, K. M., Xu, P., Ni, F., Ji, H. H., Chen, M. C., Yang, C. C., Lin, F. P., Chiu, I. M., Yu, C. Solution structure of the ligand binding domain of the fibroblast growth factor receptor: role of heparin in the activation of the receptor. Biochemistry. 2005; 44(48):15787-98. doi: 10.1021/bi051030n.
45. Powers C J, McLeskey S W, Wellstein A. Fibroblast growth factors, their receptors and signaling. Endocrine-related cancer. 2000; 7(3):165-97. doi: 10.1677/erc.0.0070165.
46. Bhuyan A K, Udgaonkar J B. Observation of multistate kinetics during the slow folding and unfolding of barstar. Biochemistry. 1999; 38(28):9158-68. doi: 10.1021/bi990285w.

### Example 2. Designing and Characterization of Stable Chimeric sFGF1-FGF21 Mutants

Fibroblast growth factor 21 (FGF21) is a crucial metabolic regulator in humans. Despite its potential in treating metabolic diseases like obesity and diabetes, it is a flawed protein with high instability and a short-acting half-life. Previous work was able to produce a novel FGF21-based fusion protein, called sFGF1-FGF21, with high thermal and chemical stability, along with notable mitogenic and metabolic activities on fibroblasts and adipocytes. However, sFGF1-FGF21 still faced some degradation during storage without the presence of phenylmethylsulfonylfluoride and required additional modulation. Herein, we proposed two new mutations on sFGF1-FGF21, called G156A and G189E, to stabilize its unstable C-terminus through local residues hydrophobic or ionic interactions to extend the storage stability of the chimeric protein. We hypothesized that these mutations would not alter the structures of the chimeric proteins, retain the wild-type sFGF1-FGF21 protein's biological activities, and improve the storage time. The result confirmed that G156A and G189E have similar secondary structures at the core through CD spectra, with a positive peak at 228 nm and a negative peak at 208-210 nm, indicating a β-sheet core. The mutants have tyrosine intrinsic fluorescence at 306 nm and a quenched tryptophan emission like sFGF1-FGF21. Both mutants still possess high thermal and chemical stability with Tm around 65.61° C. (±0.2° C.) and 65.23° C. (±0.1° C.), respectively. Their chemical stability is higher than the wild-type sFGF1-FGF21 with Cm at 3.81 M (±2.02% and 12.9%, respectively). Both mutants also have low resistance to trypsin digestion, like sFGF1-FGF21. G156A and G189E also showed no binding affinity to heparin, like sFGF1-FGF21. Additionally, both variants still possess mitogenic and metabolic (exogenous palmitate oxidation) activities, like the wild-type sFGF1-FGF21 at a concentration of 0.625 nM. However, only G156A exhibited a low number of hydrophobic pockets and core compactness like sFGF1-FGF21 through ANS binding assay with maximum fluorescence intensity at 140 nM of ANS (93.39±1.27 RFU), less G189E, which is at 200 nM of ANS (138.97±2.60 RFU). Moreover, PONDR analysis showed that G156A is more ordered at the C-terminus region than G189E, indicating that G156A may help the chimeric protein stay longer in storage conditions. Indeed, while sFGF1-FGF21 is prone to degradation in storage over time at 4° C. and 25° C., G189E improved the storage time at 4° C., and G156A improved the storage stability in both 4° C. and 25° C. conditions. The results indicate that stabilizing the C-terminus by mutating flexible glycine residues, especially near the degradation point at Arginine¹⁵⁰, improved the stability of the chimeric protein in storage while still maintaining the same desirable biological activities from sFGF1-FGF21. In conclusion, the aim was fulfilled by the making of two mutations on sFGF1-FGF21 that improve the stability of this chimeric protein, allowing the chimeric protein to be a suitable candidate for further therapeutical development to treat metabolic diseases.

In Example 1, we produced a new chimeric protein called sFGF1-FGF21 that fused the hyper-stable FGF1 variant, sFGF1, and the metabolically active FGF21 C-terminus. Through biophysical characterizations, we showed that sFGF1-FGF21 possesses similar structural characteristics to sFGF1. The fusion protein also has enhanced stability inherited from sFGF1 with higher thermal and chemical stability than wild-type counterparts. Through a series of cell-based assays, we determined that sFGF1-FGF21 also exhibits biological activities similar to sFGF1 and FGF21, specifically at lower concentrations. Such activities are mitogenic activities on NIH/3T3 fibroblasts, enhanced ATP production implying increased glucose uptake from glucose-rich media in 3T3-L1 adipocytes and increased endogenous and exogenous fatty acid oxidation capacity in 3T3-L1 adipocytes.

Despite the promising results, sFGF1-FGF21 still faces some stability issues, specifically during storage at room temperature, which could hinder the product's potential to be therapeutics to treat metabolic diseases. One of the possible causes is the very disordered flexible, and long FGF21 C-terminus portion of the protein, which is rich in glycine residues (around five residues). Glycine is a small hydrophobic that lacks sidechain structures, which exist in other natural amino acids, and thus is the most flexible amino acid that can bend freely and is found in unstructured regions of proteins [14]. Thus, glycine residues in the FGF21 C-terminus may be suitable points of degradation for the protein. We hypothesized that sFGF1-FGF21 stability in storage can increase and, consequently, improve the protein's overall stability and half-life by mutating glycine positions in the FGF21 C-terminus such as Glycine¹⁵⁶ and Glycine¹⁸⁹ to suitable residues according to their surrounding environment. For Glycine¹⁵⁶, surrounding residues are nonpolar residues proline and leucine. Thus, another hydrophobic residue like Alanine may be suitable as a replacement for Glycine¹⁵⁶ (G156A). For Glycine¹⁸⁹, surrounding residues are polar and hydrophilic with a charged residue Arginine¹⁹⁰ next by. Therefore, another charged hydrophilic residue like Glutamate is suitable for potentially making ionic interaction with nearby arginine residue and stabilizing the region (G189E). Both Glycine residues are far from the crucial Klotho-binding residues in the FGF21 C-terminal and the FGFR-binding residues in the sFGF1 region to prevent interferences with sFGF1-FGF21's interactions to its receptors and preserve its biological functions.

Herein, we provide new variants for the sFGF1-FGF21 fusion proteins with mutated glycine residues in FGF21 C-terminus, higher stability than sFGF1-FGF21 at room temperature storage but retained the wild-type sFGF1-FGF21's notable thermal and chemical stability as well as biological activities. Our results created new variants of sFGF1-FGF21 chimera with remarkable stability and comparable activities to wild-type sFGF1-FGF21 protein and wild-type FGF1 and FGF21. They can be translated into bioactive therapeutics for metabolic syndromes.

### Results and Discussion

Design, Overexpression, and Purification of sFGF1-FGF21 Mutants

The mutants of the fusion protein sFGF1-FGF21, also known as G156A and G189E, mutated the two glycine positions Glycine¹⁵⁶ and Glycine¹⁸⁹. The 6× his-tag was added into the design of both proteins (~22.836) kDa) expressed in BL21Star (DE3) bacterial strain [15]. Both mutants were designed (Genscript, USA) using a pET-21a+ vector.

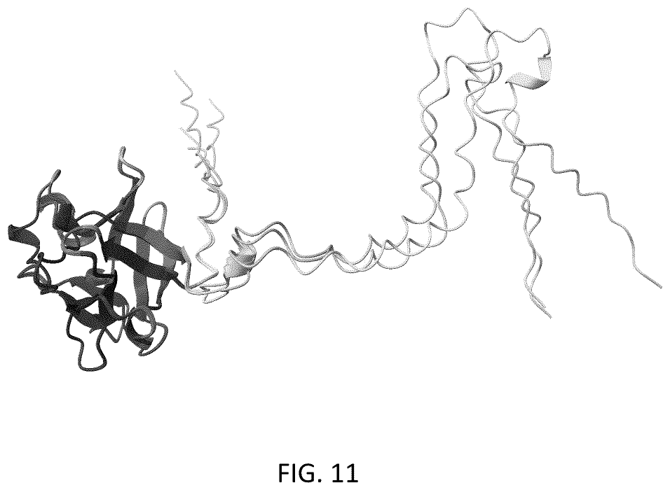

ColabFold (AlphaFold 2) was used to predict the structures of both G156A and G189E and compare them to sFGF1-FGF21. The addition of the 6×-histidine tag does not alter the conformation of the mutants, as shown in the predictions [16]. G156A and G189E predicted structures have similar features to sFGF1 and sFGF1-FGF21 and may possess similar functions as the wild-type sFGF1-FGF21 (FIG. 11). Both structures have the canonical β-trefoil core, which the predicted structure of sFG1-FGF21 also has [17]. Both mutants' C-terminus is long and flexible, like rFGF21. This region is disordered based on the low pLDDT score (between 50-70) in the predictions and the high PONDR analysis scores, which showed high disordered-ness in the C-terminus. However, there is a small but discernible difference between the G156A mutant and the G189E mutant and sFGF1-FGF21 wild-type protein. While the latter two are identical in the PONDR graph, G156A showed lower PONDR scores than these two, indicating that the G156A C-terminus may be less disordered than the wild-type sFGF1-FGF21 protein.

Transformations of G156A and G189E in XL 10 Gold and BL21Star (DE3) were successful). Similar to wild-type sFGF1-FGF21, both proteins lack any disulfide bridge, features gained from hFGF1 and sFGF1 [3, 18]. Thus, the mutants do not need specialized Rosetta-origami cells to catalyze disulfide bond formation in the cytoplasm [18]. A model bacterial expression system like BL21Star (DE3) is sufficient for expression. Indeed, both mutants transformed well with many colonies in BL21Star (DE3).

Mutants were also successfully transformed into XL 10 Gold cells to generate additional plasmids and to verify plasmid identity. The mutants-containing plasmids in XL 10 Gold were cultured and isolated via the salt-out procedure, and the quantity and quality of the mutants' plasmids were adequate after an agarose gel electrophoresis. The samples were sent to DNA sequencing (Plasmidsaurus, USA) and showed that the plasmid contains both G156A and G189E genes (SEQ ID NO: 16 and 17, respectively), indicating that the gene construction and transformation processes were successful.

G156A and G189E were overexpressed in BL21Star (DE3) cells in a small-scale format (around 10 mL of cell culture). Three separate random colonies of each mutant transformation plate were chosen to culture in an LB medium. When the culture's OD reached 0.6-0.8, the cells were induced for expression using one mM isopropyl ß-D-1-thiogalactopyranoside (IPTG) and incubated with IPTG for 5 hours. After incubation, cells were harvested and lysed using Sonifier (Branson, USA). Cell debris was separated from the lysate using centrifugation at 19,000 rpm and analyzed on sodium dodecyl sulfate-polyacrylamide gel electrophoresis (SDS-PAGE) to confirm whether the mutants were induced. We observed that in G156A, colony 3 has the best expression, and in G189E, colony 1 has the best expression. Thus, G156A colony three and G189E colony one was chosen for large-scale overexpression.

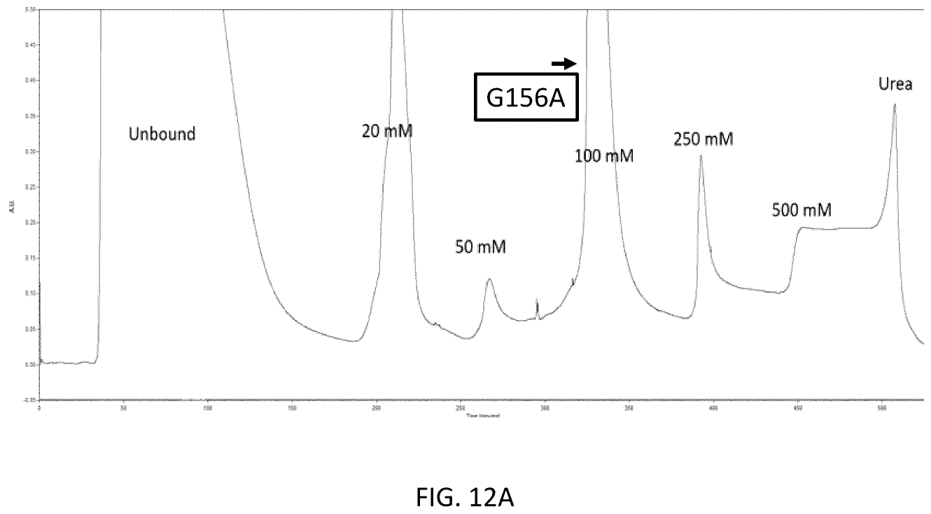

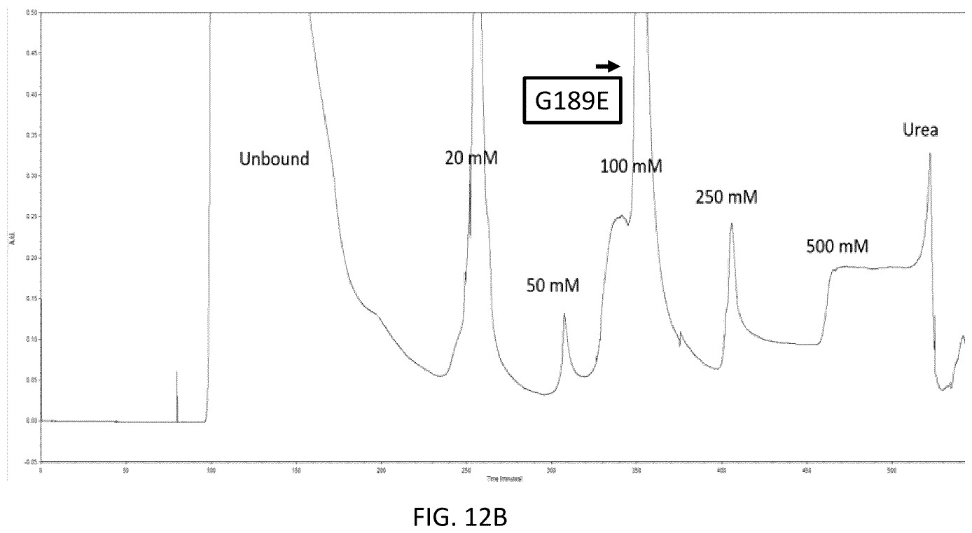

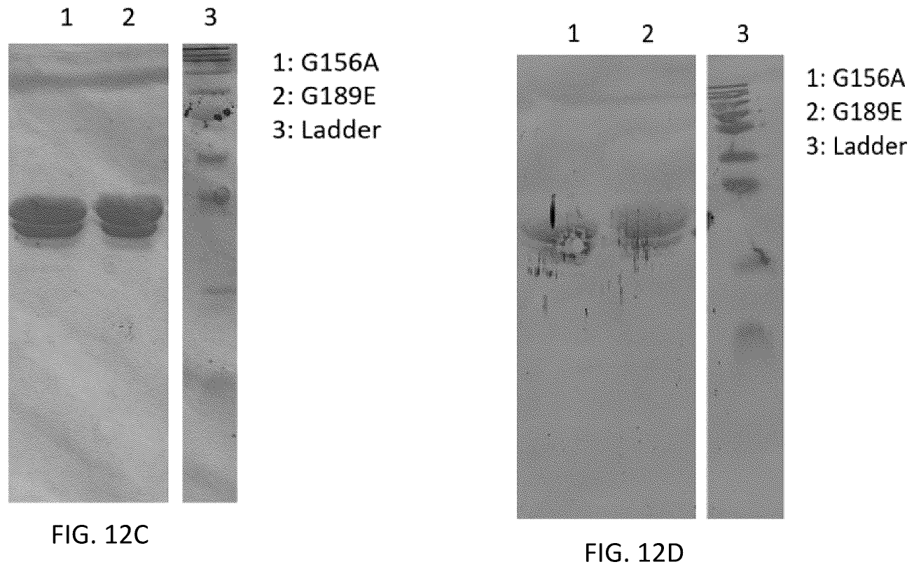

Since both G156A and G189E are his-tagged and were shown to express in BL21Star (DE3), both proteins were purified using a Ni²⁺ Sepharose column (FIGS. 12A and 12B). Both proteins were suspected to elute at around 100 mM imidazole (IMD). These observations were confirmed after an SDS-PAGE run, and the proteins were eluted in high quantities at 100 mM IMD. Samples were concentrated, and the buffer was exchanged and analyzed again on SDS-PAGE gel (FIG. 12C). Both showed a high yield of protein with a concentration of 5.3 and 5.6 mg/mL and a yield of 37.1 and 39.2 mg/L of cell culture for G156A and G189E, respectively.

It is vital to authenticate the products' identities after Ni²⁺-Sepharose purification. Therefore, a western blot was used due to the His-tags at the proteins' N-termini, like the wild-type protein sFGF1-FGF21. Using anti-his antibody is a routine method to verify if the products have the his-tags, a way to authenticate the products' identities as sFGF1-FGF21 mutants. Indeed, FIG. 12D showed that the products from Ni²⁺ Sepharose purification showed bands between 15-30 kD, indicating that both proteins have the his-tags that were designed in the mutants' sequences. Combining the DNA sequencing and the western blot results, the purified products after Ni²⁺-Sepharose purification were concluded to be sFGF1-FGF21 mutants G156A and G189E.

Biophysical Characterizations of sFGF1-FGF21

Using structure prediction and PONDR analysis, we hypothesized that the mutants may behave similarly to the sFGF1-FGF21 wild-type. In order to test this hypothesis, we utilized a variety of biophysical methods to characterize the mutant proteins in comparison to their wild-type protein, sFGF1-FGF21. Such tests may confirm our hypothesis as well as indicate that the mutation of glycine residues on the C-terminus still retains the fusion protein's biophysical characteristics and stability.

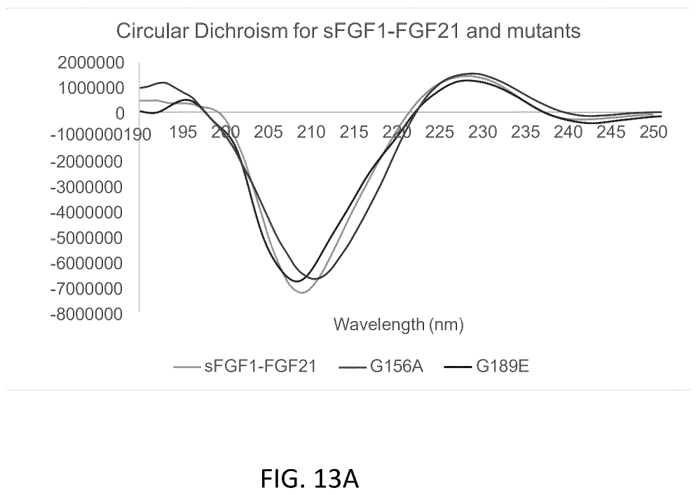

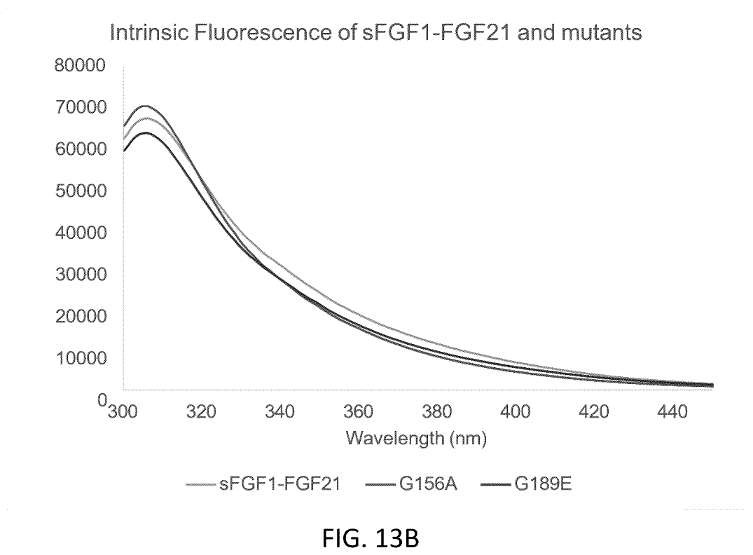

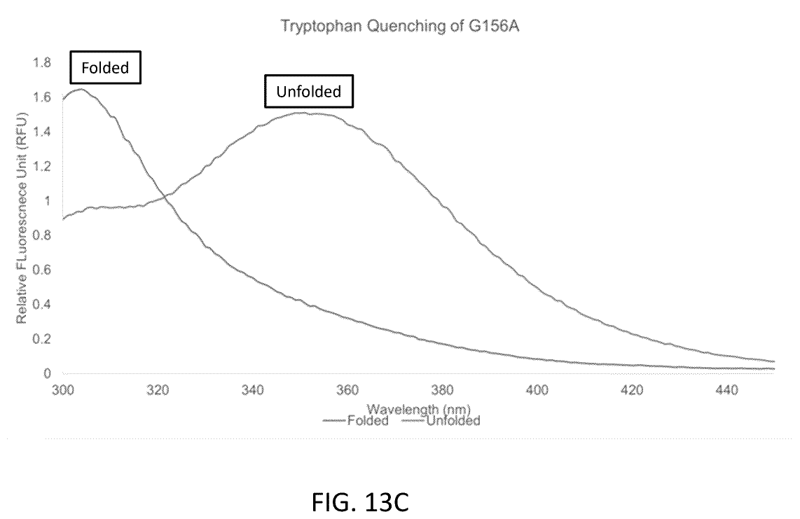

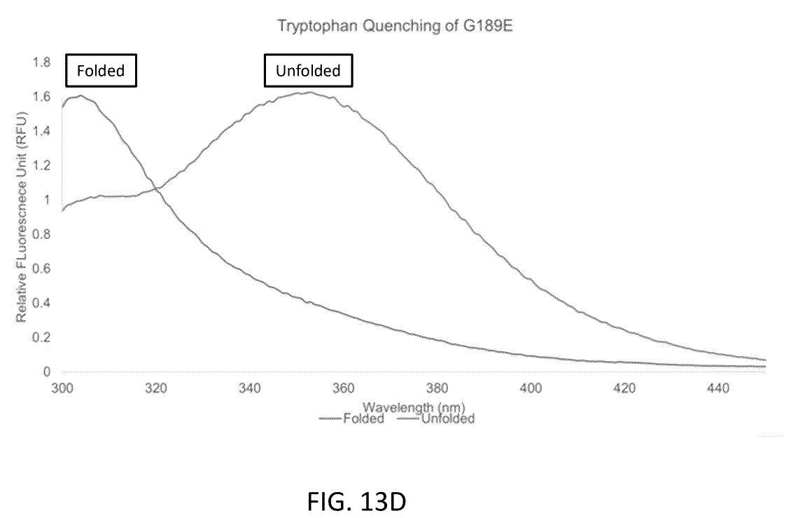

G156A and G189E mutants' circular dichroism (CD) spectra are similar to the sFGF1-FGF21 wild-type protein. All have negative peaks around 208-210 nm, which is a signature of a β-sheet core structure, and a positive peak at 228 nm, also consistent with features of a β-trefoil core (FIG. 13A). As observed in Example 1, sFGF1-FGF21 followed the typical characteristics of wtFGF1 and sFGF1 in its secondary structure. With the results of the mutants, one can see that the mutant proteins G156A and G189E also followed the CD spectra of wtFGF1 and sFGF1. This result supports our hypothesis that the mutations on sFGF1-FGF21 do not alter the protein's core structures.

G156A and G189E intrinsic fluorescence spectra also showed tyrosine emission, which peaks at 306 nm. Both mutants have the same number of tryptophan and tyrosine as sFGF1-FGF21 and can be expected to show the same fluorescence emission (FIG. 13B). Similar to sFGF1-FGF21, and also to wtFGF1 and sFGF1, the mutants' tryptophan emission was quenched due to the surrounding residues with amine/imine groups (lysine and proline) in both the proteins' conformation that can quench the tryptophan emission (FIGS. 13C and 13D). When the protein unfolds, due to the thermal or chemical denaturation process, these nitrogen-containing sidechains move away from the Tryptophan residue, allowing the residue to be exposed to solvent and an observable redshift (from 308 nm to 350 nm) in the intrinsic fluorescence spectra occurred [19] (FIGS. 13C and 13D). As both mutants showed this same phenomenon, we can utilize intrinsic fluorescence as a test to verify whether the proteins are correctly folded or not by observing the inherent fluorescence at 308 nm and 350 nm. In our results, G156A and G189E exhibited mainly tyrosine emission at 308 nm, indicating that the proteins in working buffer conditions were correctly folded, similar to sFGF1-FGF21.

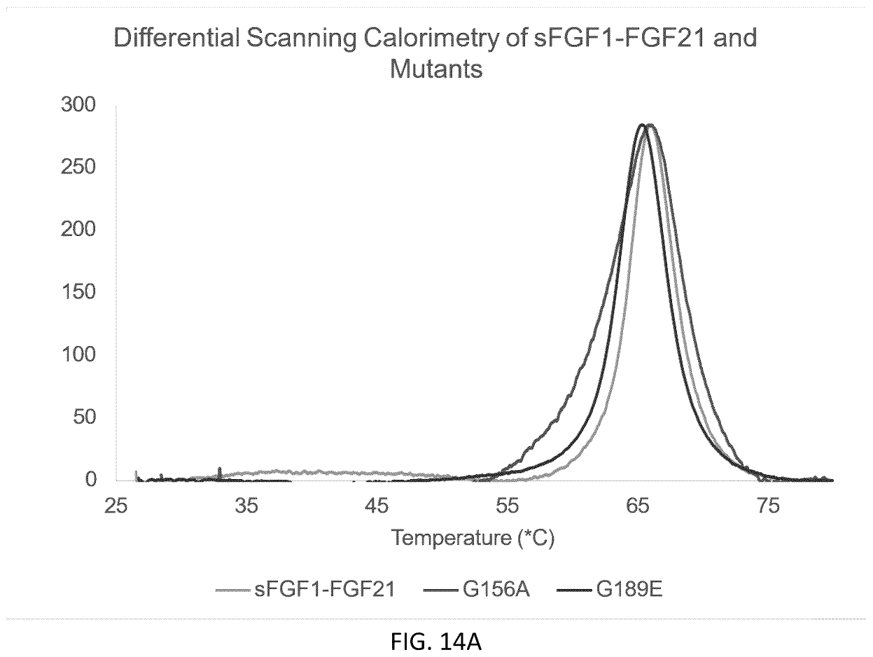

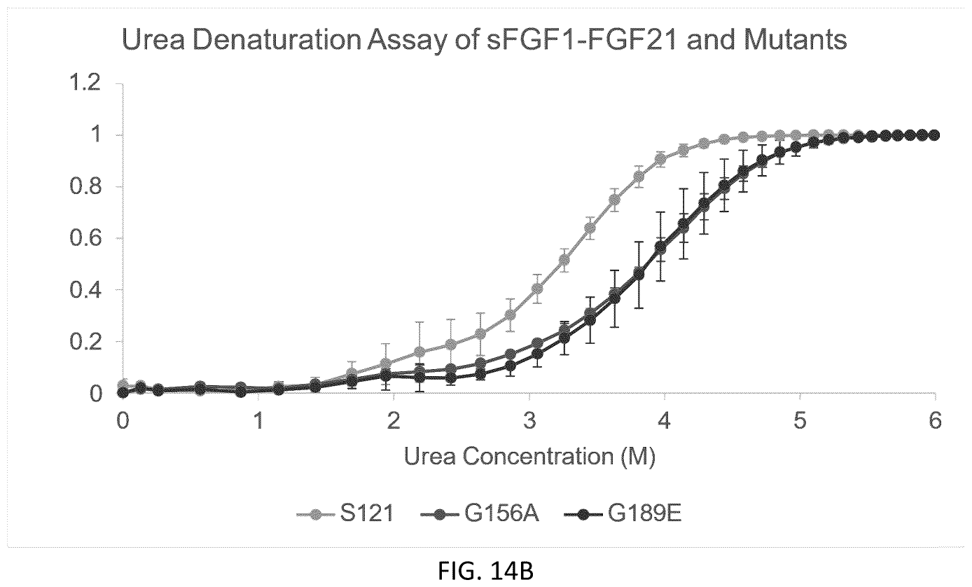

Beyond biophysical structures, sFGF1-FGF21 mutants also possess hyper-stability characteristics similar to sFGF1-FGF21 wild-type protein. Indeed, sFGF1-FGF21 showed high thermal with thermal transition midpoint (Tm) at 65.92° C. (±0.1° C.) using differential scanning calorimetry. Similarly, G156A and G189E showed Tm of around 65.61° C. (±0.2° C.) and 65.23° C. (±0.1° C.), respectively (FIG. 14A). The results indicate that both mutations still retain the chimeric protein's high thermal stability. sFGF1-FG21 has been reported to be more thermally stable than both wild-type counterparts wtFGF1 (Tm around 45.47±0.14° C.) and rFGF21 (Tm around 46.7±0.1° C.) in Example 1. With the mutants' results closely matched sFGF1-FGF21, one can agree that G156A and G189E are also more thermally stable than the wild-type counterparts, wtFGF1 and rFGF21.

As reported in Example 1, the chemical transition point (Cm) of sFGF1-FGF21 in urea-induced unfolding curves (pH=7.2) is 3.26±0.04 M and more chemically stable than both wild-type counterparts rFGF21 (Cm of 0.42±0.07 M) and wtFGF1 (1.15±0.02 M). Likewise, G156A and G189E exhibit very high resistance to denaturant-induced unfolding (8 M urea) like sFGF1-FGF21 (FIG. 14B). G156A and G189E both exhibited Cm of 3.81 M (=2.02% and 12.9%, respectively), much higher than sFGF1-FGF21 and the wild-type counterparts rFGF21 and wtFGF1. Altogether, G156A and G189E retain the necessary high thermal and chemical stability features of sFGF1-FGF21 wild-type protein and resolve the instability of wild-type rFGF21, making them ideal candidates for therapeutic developments.

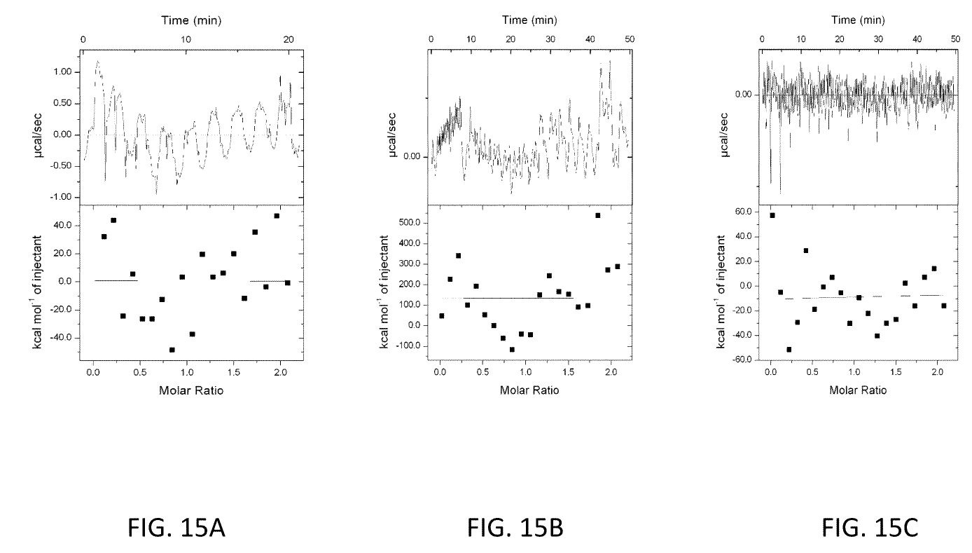

This characteristic of the fusion protein is due to having both rFGF21, which possesses low affinity to heparin [6], and sFGF1, which is designed to have low heparin affinity [17]. Therefore, sFGF1-FGF21 also has a low affinity to low-molecular-weight heparin, as reported in Example 1 (FIG. 15A). As both G156A and G189E are closely related to the wild-type protein sFGF1-FGF21 in structures and features, it is also hypothesized that both G156A and G189E will have no binding affinity to heparin. To verify this hypothesis, we utilized a standard method to measure protein-ligand binding affinity called isothermal titration calorimetry (ITC) [20]. ITC also reported no discernible interactions between low-molecular-weight heparin and G156A and G189E, confirming our hypothesis above (FIGS. 15B and 15C). With many shared biophysical features, the mutants may also function similarly to sFGF1-FGF21 wild-type protein as well.

In Example 1, sFGF1-FGF21 is shown to possess a high number of charged residues (ten lysines, seven arginines), which is higher than sFGF1 (ten lysines, five arginines), wtFGF1 (eleven lysines, six arginines) and rFGF21 (four lysines, ten arginines) [17, 20]. rFGF21 is prone to proteolysis, specifically at the C-terminus portion [21, 22]. As variants of sFGF1-FGF21, both G156A and G189E have a high number of charged residues. They also have the FGF21 C-terminus, and thus, they may be prone to trypsin digestion proteolysis like rFGF21 and sFGF1-FGF21 wild-type protein. Therefore, we utilized limited trypsin digestion to test this hypothesis about the mutants' proteolysis tendencies. As shown in Example 1, sFGF1-FGF21 has low resistance against trypsin proteolysis at low trypsin concentration through a concentration-dependent trypsin assay. A trypsin concentration of 0.00005 mg/mL was enough to cleave the majority of the protein (~75%) at 37° C. for 40 minutes and was chosen for the time-dependent trypsin assay from 0-80 minutes. Herein, we utilized the same amount of trypsin in a time-dependent trypsin digestion assay from 0-80 minutes to study the proteolysis proneness of the mutant proteins G156A and G189E.

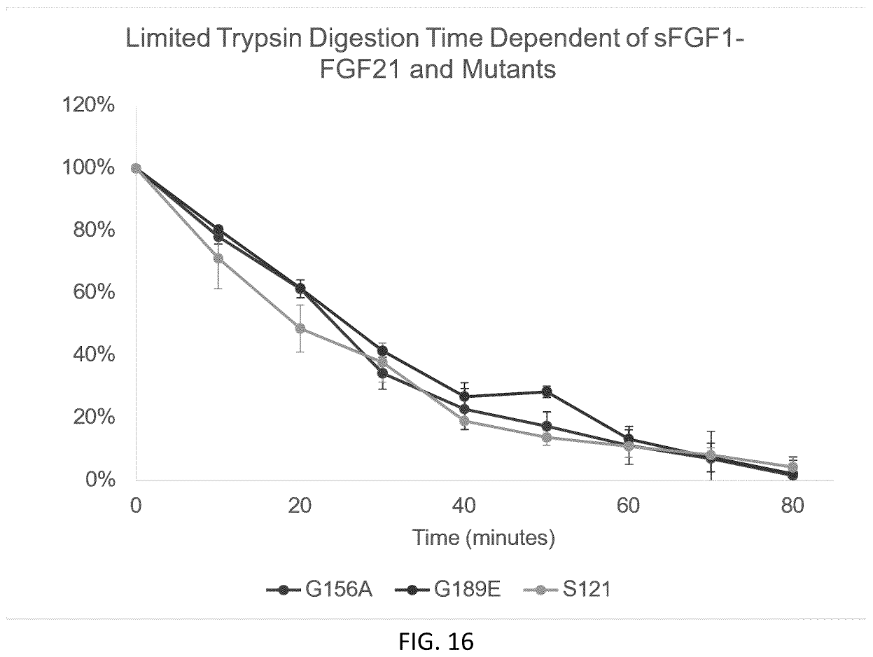

Like sFGF1-FGF21, both variants do not have resistance to trypsin digestion (FIG. 16). They are cleaved rapidly to around 2% of protein left intact (±2.46% and 2.05% for G156A and G189E, respectively). The observations are similar to sFGF1-FGF21, where only about 4.24±3.47% of the undigested protein was left after 80 minutes of digestion (FIG. 12B). The variants G156A and G189E do not have high proteolysis resistance unique to sFGF1. One of the possible causes for this lack of resistance to trypsin cleavage may rely on the FGF21-C-terminus portion of the design, which is long, flexible, and a proteolysis target [21, 22]. This FGF21 C-terminus region has at least two positively charged residues (arginine) that are accessible for trypsin. Similar to the chimera protein, both mutant proteins were cleaved to the sFGF1-like fragment (~15 kDa) of sFGF1-FGF21, which is highly resistant to proteolysis (FIG. 16). This showed that the variant proteins might only retain the resistance to proteolysis only at the sFGF 1 core and not at the C-terminus. This observation can help improve future works on the mutation of sFGF1-FGF21 that can reduce proteolysis tendencies through removing or replacing arginine residues.

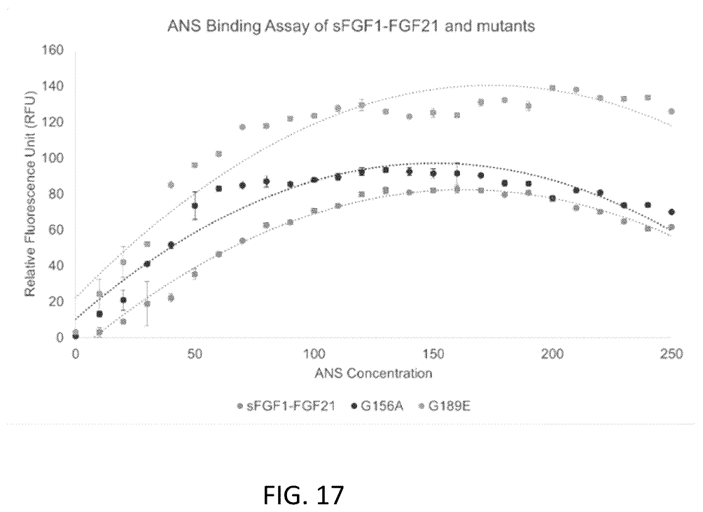

The tertiary structures of G156A and G189E can be further investigated by an 8-anilinonaphthalene-1-sulfonate (ANS) binding assay. The results can provide valuable information regarding the amount of solvent-exposed hydrophobic pockets of the proteins, which exist mainly at the proteins' cores. Such observation can be made by relying on the fluorescence intensity of a non-polar extrinsic fluorophore ANS that binds to these solvent-exposed hydrophobic pockets. The hydrophobic pockets of the proteins are directly proportional to the relative ANS fluorescence intensity, and it can elucidate how flexible the core of the proteins is [23]. As shown previously, sFGF1-FGF21's solvent-exposed hydrophobic pockets are similar to that of sFGF1, which requires a small amount of ANS to reach the maximum, around 130 nM of ANS (82.14±1.88 RFU). Similar to the wild-type sFGF1-FGF21 protein, G156A also needs a small amount of ANS to reach a maximum at 140 nM of ANS (93.39±1.27 RFU). However, G189E reached maximum fluorescence at 200 nM of ANS (138.97±2.60 RFU) (FIG. 17). The results indicated that G156A followed the same core behavior as sFGF1-FGF21 wild-type protein and has around the same amount of solvent-exposed hydrophobic pocket regions. On the other hand, G189E exhibited higher core flexibility and more hydrophobic pockets than both G156A and wild-type sFGF1-FGF21 protein. Thus, G156A, at its core, may be less flexible and more compact than G189E and resemble more closely to sFGF1-FGF21 than G189E.

Furthermore, G156A also displayed higher fluorescence intensity than sFGF1-FGF21, indicating that the substitution of Glycine¹⁵⁶ residue to a hydrophobic Alanine residue increased the hydrophobic interaction between Alanine¹⁵⁶ and nearby hydrophobic residues Phenylalanine¹⁵¹-Proline¹⁶². This hydrophobic interaction may contribute further to the stability of the chimeric protein and improve the resistance to degradation that G156A mutant showed. Together, the ANS-binding assays indicated that G156A may provide more stability to the sFGF1-FGF21 design than G189E.

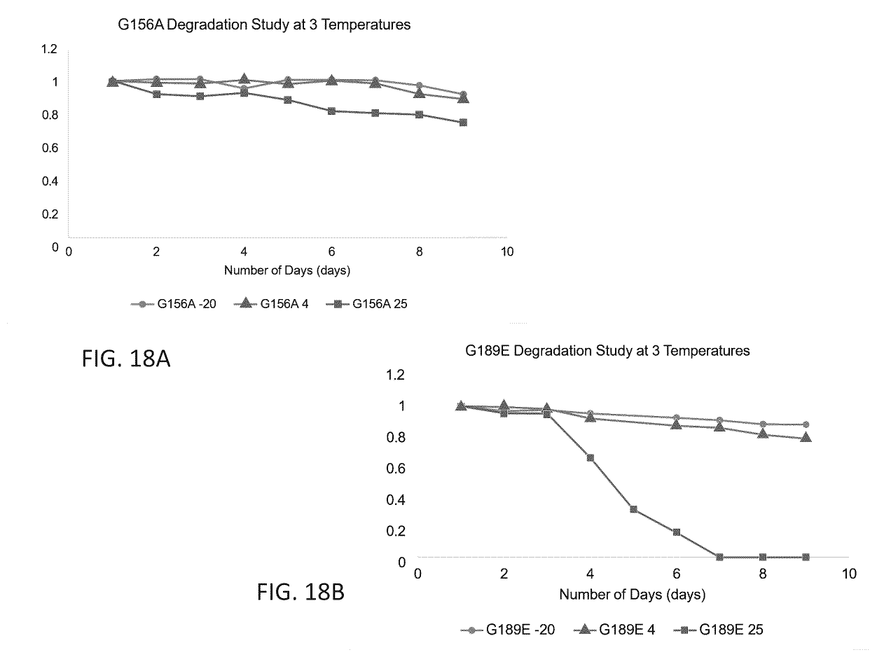

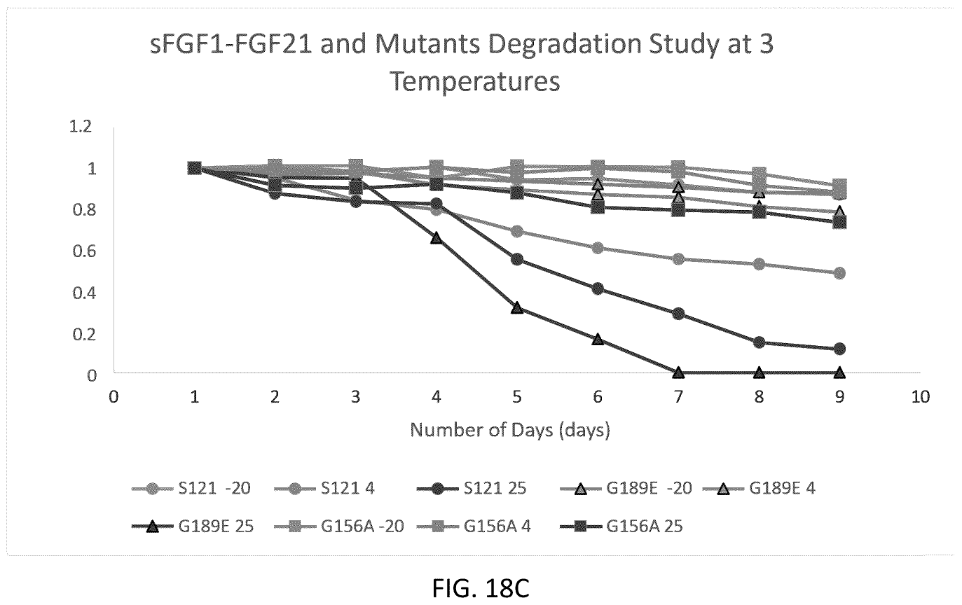

sFGF1-FGF21 possesses an unstable FGF21 C-terminus with low storage stability, as shown in Example 1. Specifically, sFGF1-FGF21 degraded at the C-terminus rapidly at 25° C., slowly at 4° C., and remained mostly intact at −20° C. The addition of 1 mM phenylmethylsulfonylfluoride helped improve sFGF1-FGF21 stability. However, we aimed to improve this storage ability permanently without the need for additional preservatives, hence the development of G156A and G189E. To test the stability of both mutant proteins, we observed the degradation patterns of the proteins over nine days in the working buffer at three different three temperatures (−20° C., 4° C., 25° C.). Samples after each day were run on SDS-PAGE and quantified using densitometric scan software (UN-SCAN-IT, Silk Scientific, USA). The percentage of intact proteins was calculated and graphed (FIGS. 18A and 18B for G156A and G189E, respectively). All trends were compared together with sFGF1-FGF21 wild-type protein for comparison (FIG. 18C).

Similar to sFGF1-FGF21, both proteins were mostly intact at −20° C. However, starting at 4° C., G156A and G189E showed notable improvement in the percentage of intact protein. While sFGF1-FGF21 only has around 48.5% of remaining intact protein after nine days at this temperature, both mutant proteins have higher percentages of 88.3% and 78.5%, respectively. G156A has the highest amount of intact protein, followed by G189E, and then the wild-type sFGF1-FGF21 protein has the least amount of intact protein. In 25° C. storage conditions, rapid degradation can be seen with the wild-type protein sFGF1-FGF21 and G189E variant. However, G156A showed a remarkable retention of intact protein at around 73.3%.

Like sFGF1-FGF21, G189E started degrading from the C-terminus and into the sFGF1 fragment, where there was no further degradation. Despite the low storage stability at room temperature (25° C.), G189E still showed significant improvement at 4° C. storage. The other mutant, G156A, showed much more improvement in storage stability in both 25° C. and 4° C. conditions. Combining the ANS assay, PONDR analysis, and degradation study results, we can see that stabilizing the C-terminus by mutating the flexible glycine residues in this region does provide additional stability to the sFGF1-FGF21 chimeric protein confirmed our hypothesis. As stated in Example 1, the point of degradation is the Arginine¹⁵⁰ position in the sFGF1-FGF21 design. It seems like mutating glycine residues around this position is more effective in making the protein last longer, as in the case of G156A. This could be due to the fact that G156A provides additional hydrophobic interactions in the regions near Arginine¹⁵⁰, adding some needed structures and ordered-ness to the area and allowing the protein to last longer, as shown in PONDR analysis. This mutation still follows a similar core compactness as that of sFGF1-FGF21 wild-type protein, as demonstrated in the ANS binding assay. On the other hand, mutating glycine in position later in the sequence seems to offer some stability for chimeric protein at 4° C. storage conditions. Still, it does not improve the disordered-ness of the region around Arginine¹⁵⁰. Thus, G189E cannot make the protein more stable in room-temperature storage conditions.

Mitogenic Activities of sFGF1-FGF21 Mutants G156A and G189E

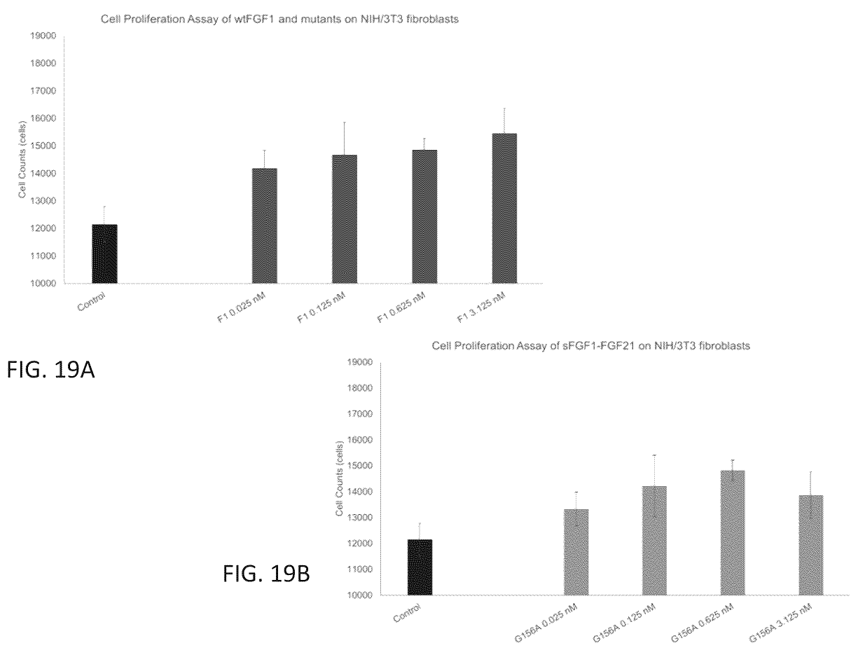

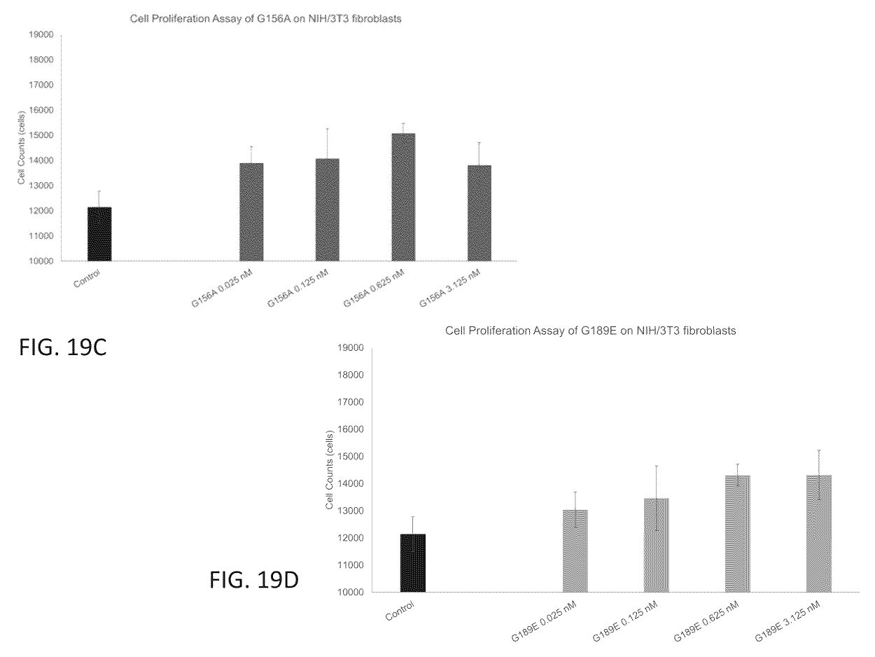

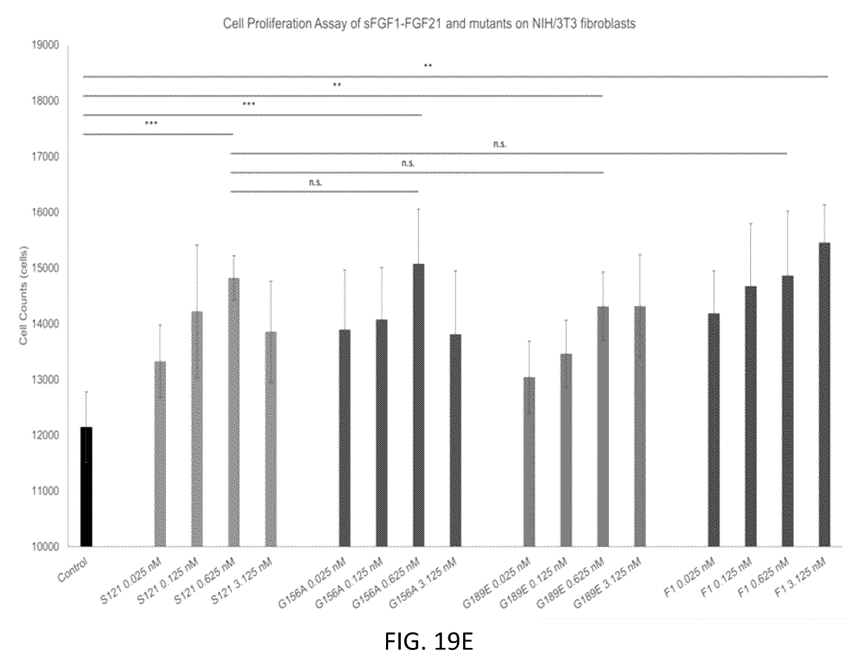

It is vital to test the biological activities of newly produced sFGF1-FGF21 mutants, G156A and G189E, to confirm whether these proteins also possess similar activities as their wild-type protein sFGF1-FGF21. Following previously identified methods for NIH/3T3 fibroblast cells, both of these proteins were used to test the cell proliferation of fibroblasts and compared to sFGF1-FGF21 and wtFGF1, a known mitogenic agent [20]. Fibroblasts were treated with different proteins (wtFGF1, sFGF1-FGF21, G156A, and G189E) at the concentrations of 0.025 nM, 0.125 nM, 0.625 nM, and 3.125 nM, and grew for 24 hours. The data demonstrated that wtFGF1 followed the typical mitogenic activities in a dose-dependent manner compared to the control (12145.3±639.1 cells) on average, peaking at 3.125 nM (15456.5±677.7 cells). This result confirmed wtFGF1 activities that were reported previously (FIG. 19A). sFGF1-FGF21 also showed fibroblast proliferative activities in low concentration, peaking at 0.625 nM (14822.5±398.8 cells). However, at higher concentrations, the chimeric protein's activities decreased (FIG. 19B), as reported previously. Similarly, G156A also showed increased mitogenic activities, peaking at 0.625 nM (15076.17±984.9 cells), and can proliferate the most cells on average (FIG. 19C). G189E also showed cell proliferative activities on NIH/3T3, peaking at both concentrations of 0.625 and 3.125 nM (14313.3±615.5 and 14318.0±918.1 cells, respectively), which is slightly different than sFGF1-FGF21 and G156A where the proliferative activities go down (FIG. 19D). However, the results of both concentrations do not statistically differ. Likewise, compared sFGF1-FGF21, G156A, and G189E at 0.625 nM, their most biologically active concentrations, do not show any statistical difference, indicating that the mitogenic activities of the mutant proteins are the same as the wild-type sFGF1-FGF21 protein (see FIG. 19E). On the other hand, when compared to the control, both sFGF1-FGF21 and G156A at 0.625 nM showed statistical differences and may indicate that the fusion protein and its variant, G156A, have notable mitogenic activities (See FIG. 19E).

Mitogenic Activities of sFGF1-FGF21 Mutants G156A and G189E

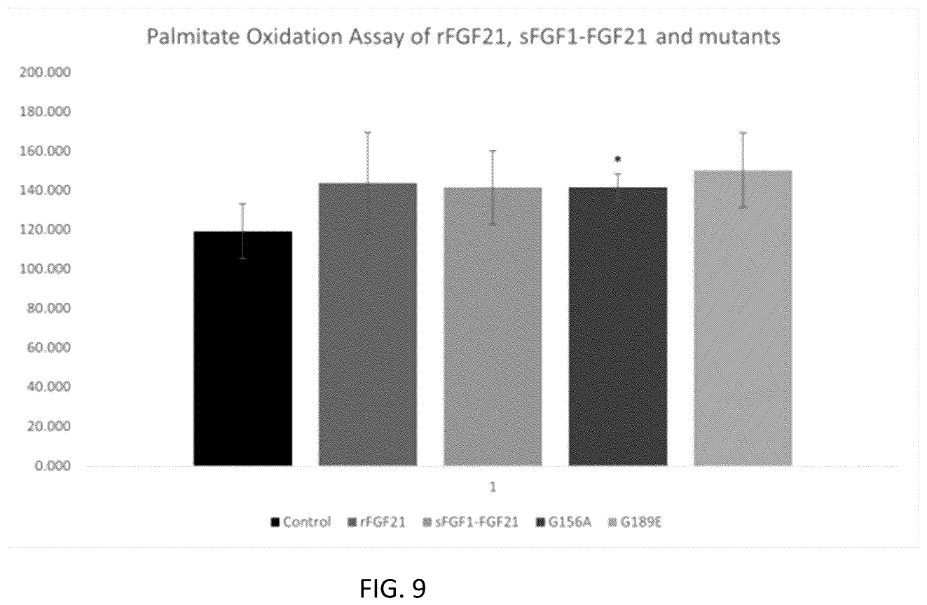

It is crucial to verify if the mutant proteins G156A and G189E retain the metabolic activities of sFGF1-FGF21 wild-type protein. sFGF1-FGF21 was reported to retain rFGF21's ability to induce ATP production and mitochondrial oxidation, which may imply glucose uptake from a high-glucose environment in 3T3-L1 adipocytes. sFGF1-FGF21 has also shown to have the ability to enhance fatty acid oxidation in adipocytes through Seahorse real-time cell metabolic analysis. Herein, we followed the same exogenous fatty acid oxidation assay, called the seahorse XF palmitate oxidation advanced assay, to test and compare the sFGF1-FGF21 wild-type protein and variants' ability to enhance fatty acid oxidation (FIG. 9). The data demonstrated that, on average, all protein treatment groups have higher OCR than control (119.1±13.9 μmol/min). Using 0.625 nM, sFGF1-FGF21, G156A, and rFGF21 showed similar maximum OCR (141.5±18.7 μmol/min, 141.6±6.8 μmol/min, and 143.9±25.6 μmol/min, respectively). rFGF21 still showed higher OCR than both sFGF1-FGF21, and also higher than G156A. G189E showed the highest OCR at 150.3±19.0 μmol/min. However, there is no statistical difference between sFGF1-FGF21, G156A, G189E, and rFGF21 OCR, indicating that the mutants may have the same metabolic activities in adipocytes as rFGF21 and sFGF1-FGF21 do and confirm our hypothesis that the mutations would not alter sFGF1-FGF21 mitogenic and metabolic activities. At the same time, it still enhances the chimeric protein's storage stability.

### Conclusions

The chimeric protein sFGF1-FGF21 variants G156A and G189E were successfully produced through a bacterial expression system. Their biophysical characteristics were delineated and showed to possess similar structural features as their wild-type protein sFGF1-FGF21. G156A mutant showed higher stability in storage at room temperature than the wild-type sFGF1-FGF21, indicating higher overall stability by reducing flexibility in chimeric protein's C-terminus region, as hypothesized. Both G156A and G189E mitogenic and metabolic activities were successfully demonstrated in this Example. The mutants possess similar mitogenic activities on NIH/3T3 fibroblasts like sFGF1-FGF21 wild-type. Likewise, their metabolic activities, like increasing exogenous fatty acid oxidation in mature 3T3-L1 adipocytes, are similar to sFGF1-FGF21 as well. In conclusion, this aim was fulfilled by producing a more stable in-storage variant of sFGF1-FGF21, with similar structural features and high thermal and chemical stability, as well as comparable biological activities to sFGF1-FGF21 wild-type protein. The results may help establish sFGF1-FGF21 as potential biomedical applications with desirable qualities to treat metabolic diseases like MAFLD and T2DM.

### Materials

The competent cells were XL10 gold (Agilent Technologies, USA), Rosetta-gami (Sigma and MP Biomedicals, LLC.), and BL21 Star DE3 (Thermo Fisher Scientific, USA). NIH/3T3 and 3T3-L1 cells were obtained from ATCC, USA. IBI Scientific (USA) supplied the Luria-Bertani (LB) broth. Cytiva (USA) supplied the heparin sepharose and nickel Sepharose resins. VWR Scientific (USA) supplied the buffer components (Na2HPO4, tris, NaH2PO4, glycine, NaCl), acrylamide, bis-acrylamide, coomassie blue, ethanol, methanol, urea crystal, acetic acid, Tween 20, glycerol, media filter units, and other supplies. Sigma and MP Biomedicals (USA) supplied phenylmethylsulphonyl fluoride (PMSF), trypsin (from bovine pancreas), and low molecular weight (~3000 Da) heparin sodium salt. Low molecular weight heparin (~3 kDa) was used for this study to avoid the polydisperse nature of heparin. Ammonium Sulfate was from EMD. Alkaline phosphatase anti-His antibody [HIS-1] is from Abcam, UK. Nitrocellulose membrane 0.2 μm was from Bio-Rad Laboratories (USA). Other materials were of analytical grade or equivalent high-quality, as described elsewhere [25]. Penicillin-streptomycin, DMEM, bovine calf serum (BCS), 0.25% trypsin in EDTA, and fetal bovine serum (FBS) were from ATCC (USA). Seahorse XF Pro, XFe24, XF HS Mini, and BioTek Cytation 5 were from Agilent (USA). SPARK microplate reader was from Tecan Life Sciences (USA). Seahorse XF Real-time ATP rate assay kit, XF palmitate oxidation stress test, XF substrate oxidation stress test kit, and accompanied medium were supplied by Agilent (USA). DAPI and Hoechst Nucleic Acid Stains 33342 were from Thermo Fisher Scientific, USA. For differentiation chemicals, insulin (Bovine) and 3-isobutyl-1-methylxanthine (IBMX) were supplied by Sigma (USA), and dexamethasone was supplied by G Biosciences (USA). Samples were made in 10 mM phosphate buffer, 150 mM NaCl, and 25 mM (NH4)2SO4 at pH 7.2 (working buffer) unless stated otherwise.

Construction and Purification of sFGF1-FGF21 Mutants (G166A and G189E)

Design of mutants for super acidic human FGF1 fused with FGF21 C-terminal (residues 161-209) (sFGF1-FGF21) at position G166 and G189, called G166A and G189E, were cloned (Genscript, USA) into the pET21a+ vectors with ampicillin resistant gene. Both mutants were transformed into highly competent bacterial cells XL 10 Gold to produce more plasmids as recommended by the manufacturer. Plasmid isolation of sFGF1-FGF21 mutants-containing XL 10 Gold cells was conducted following the manufacturer's recommendation. The resulting plasmids were run on 1% Agarose Gel Electrophoresis (AGE) to determine their existence and purity. The plasmid concentrations were determined via Nanodrop Microvolume Spectrophotometers (Thermo Fisher Scientific, USA) within the double-stranded DNA concentration estimation protocol. Plasmid was sent for DNA sequencing to confirm identity (Plasmidsaurus, USA).

BL21 Star (DE3) was used to express both proteins. Both were grown in LB media at 37° C., 240 rpm to around 0.6-0.8 optical density at 600 nm and incubated with one mM IPTG at 37° C., 240 rpm, for 5 hours. Cells were centrifuged into a pellet and resuspended in a working buffer. Afterward, induced cells were lysed on ice by Sonifier SFX150 (Branson Ultrasonics, USA) for 75 cycles with 10-second on/off pulses for a total of 30 minutes with a 15-minute break in between to let the solution cool down. The cell debris and lysate were centrifuged at 19,000 rpm by Avanti J-25 Centrifuge (Beckman Coulter, USA) for 25 minutes to separate from each other. The supernatant of sFGF1-FGF21 mutants was run over the Nickel Sepharose affinity column that was pre-equilibrated with a working buffer at one millimeter per minute. A stepwise imidazole (IMD) gradient (20-500 mM IMD) was used to elute out the product and was monitored at 280 nm. The products' purity was verified using SDS-PAGE (15%) at 200 V for 50 minutes, staining with Coomassie brilliant blue. The Bradford assay and Nanodrop Microvolume Spectrophotometers (Thermo Fisher Scientific, USA) (using the protein 280 nm concentration estimation protocol) were utilized to determine the products' concentrations [26, 27].

Predictor of Natural Disordered Regions (PONDR) Analysis of sFGF1-FGF21 and Mutants

Using the tools available at www.pondr.com, PONDR analysis was conducted. After sFGF1-FGF21 and mutant sequences were input into the appropriate space, predictor VLXT was chosen. The chosen output options were graphics, statistics, and sequence reports. The result was collected and displayed as is.

Structure prediction of sFGF1-FGF21 mutants using ColabFold (AlphaFold 2) sFGF1-FGF21 mutants' prediction was also made using the AlphaFold 2/ColabFold prediction program. Within the ColabFold notebook v1.5.2 (available at github.com/sokrypton/ColabFold), AlphaFold2_mmseqs2 was chosen. The chimeric protein sequence was input into the notebook. Other options that were chosen: the database that was searched against (msa_mode) is UniRef and Environmental (mmseqs2_uniref_env); the number of models unused (num_relax) is 0. After connecting to an available machine in AlphaFold 2, the setting was run, and a structure of the sFGF1-FGF21 mutants was produced by choosing the best model out of all five trained models' predictions in the server. Blue-colored regions are low in error estimate, red is medium, and yellow is high according to the pLDDT score.

### Western Blot

Since sFGF1-FGF21 mutants' designs have a Histidine tag at the N-terminus, a western blot was carried out to confirm the identity of the product using alkaline phosphatase anti-6× his antibody. The purified products that were thought to be sFGF1-FGF21 mutants, at concentration of 31.4 μM, were run on 15% SDS-PAGE gel for 50 minutes at 200 V. This gel was subjected to the transfer process to transfer the protein sample from gel onto the nitrocellulose membrane 0.2 μm through electrophoresis in Towbin (20% v/v methanol, 192 mM glycine, 25 mm Tris, pH 8.3). The current was set at 150V and current at 80 mA for 120 minutes. The membrane, now with the transferred protein, was soaked in 5% skim milk solutions in TBS-T solution (150 mM NaCl, 0.1% w/v Tween 20, 20 mM Tris) for 30 minutes and rinsed with TBS-T. The membrane was subjected to 0.2% BSA in TBS-T with added anti-6× antibody and incubated at room temp overnight. NBT/BCIP solution was added the following day to visualize the bands on the membrane, which represented the His tag-containing product, confirming the identity of the product as sFGF1-FGF21.

### CD and Fluorescence Spectroscopy

J-1500 Spectrophotometer (JASCO Inc., Japan) with circular dichroism detection provided the CD spectra for sFGF1-FGF21 mutants to study the secondary structures of sFGF1-FGF21 mutants. For analysis, a sample (31.4 μM) was added into a 0.1 cm path-length quartz cell using 10 mM PB and 100 mM NaCl. The setting was set at 190-250 nm at 25° C., 20 nm/min scanning speed. The procedure was repeated for ten scans, and the average data was collected. The final CD data was converted to molar ellipticity.

The tertiary structures of sFGF1-FGF21 mutants were studied through intrinsic fluorescence spectroscopy by using the Fluorescence Spectrophotometer F-2500 (Hitachi High-Tech, USA). Specifically, the investigation focused on changes in the aromatic residues, tryptophan and tyrosine, which fluoresce at 340 and 308 nm. Protein concentrations of 31.4 μM were added to the working buffer. The excitation wavelength was set at 280 nm, and the emissions were from 300-450 nm. A buffer scan was done and subtracted out of the spectra to eliminate background noise.

### 8-Anilino-1-Napthalenesulfonic Acid (ANS) Binding Assay

Fluorescence Spectrophotometer F-2500 (Hitachi High-Tech, USA) was used for the ANS binding assay. The setting was 380 nm excitation wavelength, 450-600 nm emission range, and 25 nm slit width. Protein samples (31.4 μM) in the working buffer were placed in a 10 mm quartz cuvette. Stock ANS (20 mM) was titrated into the protein mixture by 10 μM incremental addition, followed by mixing and incubation for 2 minutes at room temperature before each measurement. 520 nm fluorescence intensity was collected.

### Limited Trypsin Digestion Assay

sFGF1-FGF21 mutants' limited trypsin digestion assay was conducted in the working buffer. Eight tubes (tubes 2-9, 200 μL total volume each) contained 31.4 μM of protein, and 0.00005 mg/mL concentration of bovine trypsin were mixed. The remaining two tubes (tubes 1 and 10, 200 μL each) contained only protein (31.4 μM), and only trypsin of 0.00005 mg/mL was controlled. The experimental tubes were incubated at 37° C. All tubes have a total volume of 200 μL. Different incubation time intervals were set for each tube from 2-9:10 minutes, 20 minutes, 30 minutes, 40 minutes, 50 minutes, 60 minutes, 70 minutes, and 80 minutes. Samples were removed at a specific time, as noted above, and 10% trichloroacetic acid was added to terminate the reaction. Digested samples were run on SDS-PAGE (15%, 50 minutes at 200 V), stained with Coomassie dye. UN-ScanIT densitometric software (Silk Scientific Inc., USA) was used to digitize the gels. The percentage of undigested protein compared to control tube one was calculated based on the gel's band intensity. This percentage was plotted against incubation time intervals. The processes were done in triplicate with three separate assays.

### Differential Scanning Calorimetry (DSC)

DSC experiments were performed on differential scanning microcalorimeter MicroCal PEAQ-DSC (Malvern Panalytical Inc., Netherlands) with a temperature range from 25-80° C. with 1° C. ramping temperatures. sFGF1-FGF21 mutants were in the concentration of 31.4 μM in the working buffer. Three buffer runs were conducted before the sample run and were subtracted from the sample runs to eliminate noise. Specific heat capacity for each protein was obtained using MicroCal PEAQ-DSC software (21 CFR part 11) and plotted against the temperatures of the run. The thermal transition midpoint (melting temperature Tm) for each sample was calculated and recorded.

Isothermal Titration Calorimetry (ITC) sFGF1-FGF21 mutants' binding interaction to cofactor heparin was measured using a MicroCal iTC200 microcalorimeter (Malvern Panalytical Inc., Netherlands). To maintain a 1:8 protein: ligand ratio, the degassed heparin was at a concentration of 251.2 μM, and the degassed sFGF1-FGF21 mutants were at a concentration of 31.4 μM. The heparin injection volume was 2 μL for a total of 20 titrations and was conducted at 25° C. Origin software (Malvern Panalytical Inc., Netherlands) was used to analyzed the data.

### Equilibrium Unfolding Using Chemical Denaturant.

To study the effect of chemical denaturants such as urea on sFGF1-FGF21 mutants, a J-1500 Spectrophotometer (JASCO Inc., Japan) was used. Proteins (31.4 μM in the working buffer) were titrated with 8 M urea. This denaturation process was tracked using the spectrophotometer's automatic scanning titrations with fluorescence detection. The excitation wavelength was set at 280 nm; emission wavelengths were set from 300-450 nm. The fluorescence measurements under increasing urea concentrations from 0-6 M were recorded, and a ratio between 305 and 350 nm peaks was calculated for each urea concentration. The fraction of protein unfolded in percentage was calculated for each urea concentration and plotted for each protein as reported elsewhere [28]. The chemical transition midpoint (Cm) for each sample was recorded.

Stability of rFGF21 in Storage Over Time

To investigate sFGF1-FGF21 mutants' stability in storage, we designed an experiment in which three different storage conditions were set up, with three temperatures: −20° C., 4° C., and 25, and the protein was stored in these conditions over nine days. Samples of the protein, at each temperature, were taken every day for nine days. Samples were analyzed on SDS-PAGE (15%), and gels were digitized through densitometric scanning software UN-SCAN-IT (Silk Scientific, USA) for all 9 days. Degraded and intact protein levels for each day were calculated based on a comparison with protein on the first day in percentage and plotted as days versus percentage of intact protein.

### Culturing NIH/3T3 Fibroblasts

NIH/3T3 fibroblasts from mice were grown in culture flasks (75 cm²) by seeding approximately 250,000-300,000 cells stored in N2 gas in 15-30 mL of complete media (90% DMEM, 10% BCS, 1% penicillin-streptomycin) at 37° C. in 5% CO2 incubator until cells were attached at the bottom the flasks, formed spindle shapes and confluence at around 70-90%. Cells were then removed from the media, washed twice with PBS (50 mM NaCl and 10 mM phosphate buffer), then incubated with 0.25% trypsin in EDTA for 5-7 minutes at 37° C. in 5% CO2 incubator until cells were detached from the bottom of the flasks. Cells were harvested, centrifuged at 500 rpm for 7 minutes, removed trypsin-containing media, and washed with PBS before being counted using a SPARK machine and trypan blue dye. Cells were diluted into a concentration of 200,000-300,000 cells/mL using warm complete media and seeded into new culture flasks for a 2^{nd} passage and grow into 70-90% confluence. The passage process took place for a 3^{rd} time before any downstream assays were conducted.

### Cell Proliferation Assay

NIH/3T3 fibroblasts were passaged at least three times and grown to 70-90% confluence. Cells were then synchronized by media starvation (incubating with DMEM only with no growth serum) for 12-15 hours at 37° C. in a 5% CO2 incubator. 96-well microplates were coated with PDL (3-10 μg/cm² or around two μg/well) for 12-15 hours to enhance cell attachments, then washed with 100 μL/well of PBS twice before being used. After the cell phase synchronization process, cells were washed with PBS twice, then detached using 0.25% trypsin in EDTA for 5-7 minutes at 37° C. in a 5% CO2 incubator, centrifuged at 500 rpm for 10 minutes to separate pellet from trypsin-containing media. Pellets were reconstituted into solution with complete media again (1 mL), and cell numbers were counted using SPARK cell counter and trypan blue dye. Cells were diluted into 200,000 cells/mL with warm complete media before being seeded into PDL-coated wells in the 96-well microplate in 50 μL volumes for a total amount of 10,000 cells/well. Cells were then treated with purified proteins as outlined in Example 1 (sFGF1-FGF21, sFGF1-FGF21 mutants, rFGF21) of various concentrations (0-15.625 nM) as shown in the result section, along with a control section where no protein was added. The warm complete media were added until a total volume of 100 μL for each well was reached. Cells were shaken gently for 1 minute to mix all components before being incubated at 37° C. in a 5% CO2 incubator for 12 hours. Once the incubation period ended, plates were removed from the incubator, and Hoechst 3332 (stock 20 mM) was to each well to a final concentration of 4 μM per well (~120 uL of total volume). The plate was incubated with dye for 15-30 minutes before being analyzed by BioTek Cytation 5 (parameters followed by manufacturer instructions) in DAPI reading mode. Total cell counts per well were collected and analyzed. Average cell counts per group (specific protein+ concentration) were graphed and analyzed using ANOVA and Bonferroni post-hoc test.

### 3T3-L1 Culture and Differentiation

3T3-L1 cells were seeded in a culture flask, similar to NIH/3T3 fibroblasts. After 1^{st} passage, cells were counted using SPARK with trypan blue and diluted into 50000 cells/mL for around 10 mL with complete media. The cells were seeded into PDL-plated XF Pro 96 well-plate (coated overnight at two μg/well as described above) at the concentration of 5000 cells/well in 100 μL volume with complete media. Cells were incubated at 37° C. in a 5% CO2 incubator until they reached 70% confluence (media changed once every three days). Once cells were at 70% confluence, cells were cultured for an additional 24-36 hours (day 0), then media were carefully removed from cells, and new differentiation media was added (90% DMEM, 10% FBS, one μM Dexamethasone, 0.5 mM IMBX, 10.0 μg/mL insulin) and incubated with the cells for 72 hours (day 1-3). The maintenance media (90% DMEM, 10% FBS, 10.0 μg/mL insulin) was added to the cells for another 72 hours (days 4-6). Afterward, only media with 90% DMEM and 10% FBS was added into cells, changing every 72 hours (days 7-9). After days 10-14, the fully matured adipocytes were obtained. Differentiation progress of the adipocytes was documented every two days over 14 days to observe the change in cell shapes and the fat accumulation of the cells to confirm adipocyte maturity.

### Exogenous Fatty Acid/Palmitate Oxidation Assay

Mature 3T3-L1 were starved in high glucose DMEM with 1% FBS and 0.5 mM L-carnitine for 12-15 hours. At the same time, cells were treated with purified proteins (sFGF1-FGF21, sFGF1-FGF21 mutants, rFGF21) as described in Example 1 at one concentration of 0.625 nM (as determined in real-time ATP assay) at 37° C. in 5% CO2 incubator. The appropriate cartridge was moistened in XF calibrant overnight in a humid non-CO2 incubator at 37° C. overnight. The cartridge was replaced with a new XF calibrant the following day and incubated for another hour in a humid non-CO2 incubator at 37° C. XF Palmitate media (XF DMEM+XF Glucose+L-carnitine) was made along with the modulators from XF palmitate oxidations tress test kit (oligomycin, antimycin, rotenone AA, etomoxir, FCCP) as instructed by the manufacturer then loaded into the cartridge for XF Pro calibration (half should be with etomoxir/inhibited, half should be without etomoxir/uninhibited). The culture plate was removed from the incubator after 12 hours and washed the media with XF palmitate media twice, then incubated for 1 hour in a humid non-CO2 incubator at 37° C. to remove CO2 and replaced with fresh warm XF Media. Fifteen minutes before the actual run, 30 L of Palmitate: BSA or BSA was added into the wells for a final concentration of 167 μM of Palmitate in the wells. The plate was loaded into the machine and run. Data was collected and analyzed using Seahorse Analytics and graphed. Data comparisons were made using ANOVA and Bonferroni post-hoc test.

### Data Analysis

For in vitro studies, the experiments were performed with at least five wells (n=5) or more, as stated in the figure legends. Statistical details are also provided in the figure legends. Data was collected and analyzed using one-factor ANOVA, followed by the Bonferroni test for multiple comparisons. Differences between the two groups were analyzed using unpaired two-tailed student t-tests. Data are expressed as mean±s. d.

### REFERENCES

1. Spann R A, Morrison C D, den Hartigh L J. The Nuanced Metabolic Functions of Endogenous FGF21 Depend on the Nature of the Stimulus, Tissue Source, and Experimental Model. Frontiers in endocrinology. 2022; 12:802541. doi: 10.3389/fendo.2021.802541.
2. Phan P, Saikia B B, Sonnaila S, Agrawal S, Alraawi Z, Kumar TKS, et al. The Saga of Endocrine FGFs. Cells. 2021; 10(9):2418. doi: 10.3390/cells10092418.
3. Zhu L, Zhao H, Liu J, Cai H, Wu B, Liu Z, et al. Dynamic folding modulation generates FGF21 variant against diabetes. EMBO reports. 2021; 22(1):e51352. doi: 10.15252/embr.202051352.
4. Abdelmalek M F, Sanyal A J, Nakajima A, Neuschwander-Tetri B A, Goodman Z D, Lawitz E J, et al. Pegbelfermin in Patients With Nonalcoholic Steatohepatitis and Compensated Cirrhosis (FALCON 2): A Randomized Phase 2b Study. Clin Gastroenterol Hepatol. 2023. doi: 10.1016/j.cgh.2023.04.012.
5. Adams A C, Halstead C A, Hansen B C, Irizarry A R, Martin J A, Myers S R, et al. LY2405319, an Engineered FGF21 Variant, Improves the Metabolic Status of Diabetic Monkeys. PloS one. 2013; 8(6):e65763. doi: 10.1371/journal.pone.0065763.
6. Kharitonenkov A, Shiyanova T L, Koester A, Ford A M, Micanovic R, Galbreath E J, et al. FGF-21 as a novel metabolic regulator. The Journal of clinical investigation. 2005; 115(6):1627-35. doi: 10.1172/JCI23606.
7. Charles E D, Neuschwander-Tetri B A, Pablo Frias J, Kundu S, Luo Y, Tirucherai G S, et al. Pegbelfermin (BMS-986036), PEGylated FGF21, in Patients with Obesity and Type 2 Diabetes: Results from a Randomized Phase 2 Study. Obesity (Silver Spring, Md). 2019; 27(1):41-9. doi: 10.1002/oby.22344.
8. Kaufman A, Abuqayyas L, Denney W S, Tillman E J, Rolph T. AKR-001, an Fc-FGF21 Analog, Showed Sustained Pharmacodynamic Effects on Insulin Sensitivity and Lipid Metabolism in Type 2 Diabetes Patients. Cell reports Medicine. 2020; 1(4):100057. doi: 10.1016/j.xcrm.2020.100057.
9. Zhu S, Ma, L., Wu, Y., Ye, X., Zhang, T., Zhang, Q., Rasoul, L. M., Liu, Y., Guo, M., Zhou, B., Ren, G., Li, D. FGF21 treatment ameliorates alcoholic fatty liver through activation of AMPK-SIRT1 pathway. Acta biochimica et biophysica Sinica. 2014; 46(12):1041-8. doi: 10.1093/abbs/gmu097.
10. Liu Y L, Zhao C Q, Xiao J, Liu L M, Zhang M, Wang C L, et al. Fibroblast growth factor 21 deficiency exacerbates chronic alcohol-induced hepatic steatosis and injury. Scientific Reports. 2016; 6. doi: 10.1038/srep31026.
11. Phan P, Ternier G, Edirisinghe O, Kumar T K S. Exploring endocrine FGFs-structures, functions and biomedical applications. Int J Biochem Mol Biol. 2024; 15(4):68-99.
12. Zhen E Y, Jin Z, Ackermann B L, Thomas M K, Gutierrez J A. Circulating FGF21 proteolytic processing mediated by fibroblast activation protein. The Biochemical journal. 2016; 473(5):605-14. doi: 10.1042/BJ20151085.
13. Kharitonenkov A, Beals J M, Micanovic R, Strifler B A, Rathnachalam R, Wroblewski V J, et al. Rational design of a fibroblast growth factor 21-based clinical candidate, LY2405319. PloS one. 2013; 8(3):e58575. doi: 10.1371/journal.pone.0058575.
14. Senthil R, Usha S, Saravanan K M. Importance of Fluctuating Amino Acid Residues in Folding and Binding of Proteins. Avicenna journal of medical biotechnology. 2019; 11(4):339-43.
15. López-Cano A, Martínez-Miguel M, Guasch J, Ratera I, Arís A, Garcia-Fruitós E. Exploring the impact of the recombinant Escherichia coli strain on defensins antimicrobial activity: BL21 versus Origami strain. Microbial cell factories. 2022; 21(1):77. doi: 10.1186/s12934-022-01803-7.
16. Mirdita M, Schütze K, Moriwaki Y, Heo L, Ovchinnikov S, Steinegger M. ColabFold: making protein folding accessible to all. Nature Methods. 2022; 19(6):679-82. doi: doi: 10.1038/s41592-022-01488-1.
17. Agrawal S. Design and characterization of FGF-1 mutant(s) with increased stability and enhanced bioactivity. Chemistry and Biochemistry: University of Arkansas; 2020. p. 52.
18. Berkmen M. Production of disulfide-bonded proteins in <i>Escherichia coli</i>. Protein Expression and Purification. 2012; 82(1):240-51. doi: 10.1016/j.pep.2011.10.009.
19. Agrawal S, Govind Kumar V, Gundampati R K, Moradi M, Kumar T K S. Characterization of the structural forces governing the reversibility of the thermal unfolding of the human acidic fibroblast growth factor. Scientific reports. 2021; 11(1):15579. doi: 10.1038/s41598-021-95050-2.
20. Kerr R, Agrawal S, Maity S, Koppolu B, Jayanthi S, Suresh Kumar G, et al. Design of a thrombin resistant human acidic fibroblast growth factor (hFGF1) variant that exhibits enhanced cell proliferation activity. Biochemical and biophysical research communications. 2019; 518(2):191-6. doi: 10.1016/j.bbrc.2019.08.029.
21. Dunshee D R, Bainbridge T W, Kljavin N M, Zavala-Solorio J, Schroeder A C, Chan R, et al. Fibroblast Activation Protein Cleaves and Inactivates Fibroblast Growth Factor 21. The Journal of biological chemistry. 2016; 291(11):5986-96. doi: 10.1074/jbc.M115.710582.
22. Gillum M P, Potthoff M J. FAP finds FGF21 easy to digest. The Biochemical journal. 2016; 473(9). doi:10.1042/BCJ20160004.
23. Davis J E, Alghanmi A, Gundampati R K, Jayanthi S, Fields E, Armstrong M, et al. Probing the role of proline-135 on the structure, stability, and cell proliferation activity of human acidic fibroblast growth factor. Archives of Biochemistry and Biophysics. 2018; 654:115-25. doi: 10.1016/j.abb.2018.07.017.
24. Kumar V G, Agrawal S, Kumar T K S, Moradi M. Mechanistic Picture for Monomeric Human Fibroblast Growth Factor 1 Stabilization by Heparin Binding. Journal of Physical Chemistry B. 2021; 125(46):12690-7. doi: 10.1021/acs.jpcb.1c07772.
25. Phan P, Sonnaila, S., Ternier, G., Edirisinghe, O., Okoto, P. S., Kumar, T. K. S. Overexpression and Purification of Mitogenic and Metabolic Fibroblast Growth Factors. Methods in molecular biology (Clifton, NJ). 2024; 2762:151-81. doi: 10.1007/978-1-0716-3666-4_10.
26. Hung K W, Kumar, T. K. S., Kathir, K. M., Xu, P., Ni, F., Ji, H. H., Chen, M. C., Yang, C. C., Lin, F. P., Chiu, I. M., Yu, C.,. Solution structure of the ligand binding domain of the fibroblast growth factor receptor: role of heparin in the activation of the receptor. Biochemistry. 2005; 44(48):15787-98. doi: 10.1021/bi051030n.
27. Powers C J, McLeskey S W, Wellstein A. Fibroblast growth factors, their receptors and signaling. Endocrine-related cancer. 2000; 7(3):165-97. doi: 10.1677/erc.0.0070165.
28. Bhuyan A K, Udgaonkar J B. Observation of multistate kinetics during the slow folding and unfolding of barstar. Biochemistry. 1999; 38(28):9158-68. doi: 10.1021/bi990285w

What is claimed:

1. A fusion protein comprising:
a) a fibroblast growth factor 1 (FGF1) peptide having at least 95% sequence identity to SEQ ID NO: 1; and
b) a fibroblast growth factor 21 (FGF21) peptide having at least 95% sequence identity to SEQ ID NO: 3 or a fragment thereof comprising 30-200 amino acid residues.

2. The fusion protein of claim 1, wherein the FGF21 peptide comprises at least one substitution mutation at a residue selected from residues G169 and G202 of SEQ ID NO: 3.

3. The fusion protein of claim 2, wherein the FGF21 peptide comprises
a) the substitution mutation at residue G169 and the substitution mutation is G169A;
b) the substitution mutation at residue G202 and the substitution mutation is G202E;
c) SEQ ID NO: 7;
d) SEQ ID NO: 8; or
e) SEQ ID NO: 9.

4. The fusion protein of claim 1, wherein the FGF21 peptide has at least 95% sequence identity to a C-terminal fragment of FGF21 selected from the group consisting of SEQ ID NO: 6, SEQ ID NO: 7, SEQ ID NO: 8, and SEQ ID NO: 9.

5. The fusion protein of claim 1, wherein the FGF1 peptide comprises at least one substitution mutation at a residue selected from residues Q41, S48, H94, K113, and R123 of SEQ ID NO: 1; and, wherein:
a) the substitution mutation at residue Q41 is selected from Q41P, Q41F, Q41M, Q41Y, Q41W, Q41I, Q41L, Q41V, and Q41A;
b) the substitution mutation at residue S48 is selected from S48L, S48A, S48V, S48P, S48T, S48M, S48I, S48F, S48Y, and S48W;
f) the substitution mutation at residue H94 is selected from H94S, H94T, H94K, H94R, and H94Y;
g) the substitution mutation at residue K113 is selected from K113N, K113Q, K113S, K113T, K113R, and K113Y;
h) the substitution mutation at residue R123 is selected from R123E and R123D; or
i) any combination of (a)-(e).

6. The fusion protein of claim 5, wherein the FGF1 peptide comprises the substitution mutations Q41P, S48L, H94S, K113N, and R123E, and wherein the FGF1 peptide has at least 95% sequence identity to SEQ ID NO: 2.

7. The fusion protein of claim 1, wherein:
a) the FGF1 peptide comprises SEQ ID NO: 2 and the FGF21 peptide comprises SEQ ID NO: 6;
b) the FGF1 peptide comprises SEQ ID NO: 2 and the FGF21 peptide comprises SEQ ID NO: 7;
c) the FGF1 peptide comprises SEQ ID NO: 2 and the FGF21 peptide comprises SEQ ID NO: 8; or
d) the FGF1 peptide comprises SEQ ID NO: 2 and the FGF21 peptide comprises SEQ ID NO: 9.

8. The fusion protein of claim 7, wherein the fusion protein has at least 95% sequence identity to SEQ ID NO: 10, SEQ ID NO: 11, SEQ ID NO: 12, or SEQ ID NO: 13.

9. The fusion protein of claim 1, further comprising a protein tag.

10. The fusion protein of claim 9, wherein the fusion protein has at least 95% sequence identity to SEQ ID NO: 14.

11. A pharmaceutical composition comprising the fusion protein of claim 1 and a pharmaceutically acceptable carrier.

12. A polynucleotide encoding the fusion protein of claim 1.

13. The polynucleotide of claim 12, wherein the polynucleotide is codon optimized for expression in a cell.

14. The polynucleotide of claim 13, wherein the polynucleotide comprises SEQ ID NO: 15, SEQ ID NO: 16, or SEQ ID NO: 17.

15. A construct comprising the polynucleotide of claim 12 operably linked to a promoter.

16. A wound dressing comprising the fusion protein of claim 1.

17. A method of treating a condition in a subject, the method comprising administering the fusion protein of claim 1 to the subject.

18. The method of claim 17, wherein the condition is type 2 diabetes, insulin resistance, gestational diabetes, hyperglycemia, obesity, heart disease, stroke, nonalcoholic fatty liver disease (NAFLD), nonalcoholic steatohepatitis (NASH), alcoholic fatty liver disease, a wound, tissue or organ damage, a fractured bone, a gastric ulcer, ulcerative colitis, a scar, a nerve injury, a burn, hair loss, osteoporosis, a cardiovascular disorder, a cosmetic condition, ischemia, a retinal disorder, a muscular disorder, an arterial disease, an age-related disorder, osteoarthritis, or acute renal failure.

19. The method of claim 17, wherein the subject is a human.

20. The method of claim 17, wherein administration is topical, oral, subcutaneous or intravenous.


SEQUENCE LISTING (18 sequences)

SEQ ID NO: 1 - protein, 141 aa, Homo sapiens
MFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 141 aa, synthetic construct
MFNLPPGNYKKPKLLYCSNGGHFLRILPDGTVDGTRDRSDPHIQLQLLAESVGEVYIKSTETGQYLAMDTDGLLYGSQTPNEECLFLERLEENSYNTYISKKHAEKNWFVGLNKNGSCKRGPETHYGQKAILFLPLPVSSD

SEQ ID NO: 3 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 4 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 5 - protein, 188 aa, synthetic construct
MHHHHHHHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 6 - protein, 49 aa, Homo sapiens
PARFLPLPGLPPALPEPPGILAPQPPDVGSSDPLSMVGPSQGRSPSYAS

SEQ ID NO: 7 - protein, 49 aa, synthetic construct
PARFLPLPALPPALPEPPGILAPQPPDVGSSDPLSMVGPSQGRSPSYAS

SEQ ID NO: 8 - protein, 49 aa, synthetic construct
PARFLPLPGLPPALPEPPGILAPQPPDVGSSDPLSMVGPSQERSPSYAS

SEQ ID NO: 9 - protein, 49 aa, synthetic construct
PARFLPLPALPPALPEPPGILAPQPPDVGSSDPLSMVGPSQERSPSYAS

SEQ ID NO: 10 - protein, 190 aa, synthetic construct
MFNLPPGNYKKPKLLYCSNGGHFLRILPDGTVDGTRDRSDPHIQLQLLAESVGEVYIKSTETGQYLAMDTDGLLYGSQTPNEECLFLERLEENSYNTYISKKHAEKNWFVGLNKNGSCKRGPETHYGQKAILFLPLPVSSDPARFLPLPGLPPALPEPPGILAPQPPDVGSSDPLSMVGPSQGRSPSYAS

SEQ ID NO: 11 - protein, 190 aa, synthetic construct
MFNLPPGNYKKPKLLYCSNGGHFLRILPDGTVDGTRDRSDPHIQLQLLAESVGEVYIKSTETGQYLAMDTDGLLYGSQTPNEECLFLERLEENSYNTYISKKHAEKNWFVGLNKNGSCKRGPETHYGQKAILFLPLPVSSDPARFLPLPALPPALPEPPGILAPQPPDVGSSDPLSMVGPSQGRSPSYAS

SEQ ID NO: 12 - protein, 190 aa, synthetic construct
MFNLPPGNYKKPKLLYCSNGGHFLRILPDGTVDGTRDRSDPHIQLQLLAESVGEVYIKSTETGQYLAMDTDGLLYGSQTPNEECLFLERLEENSYNTYISKKHAEKNWFVGLNKNGSCKRGPETHYGQKAILFLPLPVSSDPARFLPLPGLPPALPEPPGILAPQPPDVGSSDPLSMVGPSQERSPSYAS

SEQ ID NO: 13 - protein, 190 aa, synthetic construct
MFNLPPGNYKKPKLLYCSNGGHFLRILPDGTVDGTRDRSDPHIQLQLLAESVGEVYIKSTETGQYLAMDTDGLLYGSQTPNEECLFLERLEENSYNTYISKKHAEKNWFVGLNKNGSCKRGPETHYGQKAILFLPLPVSSDPARFLPLPALPPALPEPPGILAPQPPDVGSSDPLSMVGPSQERSPSYAS

SEQ ID NO: 14 - protein, 196 aa, synthetic construct
MHHHHHHFNLPPGNYKKPKLLYCSNGGHFLRILPDGTVDGTRDRSDPHIQLQLLAESVGEVYIKSTETGQYLAMDTDGLLYGSQTPNEECLFLERLEENSYNTYISKKHAEKNWFVGLNKNGSCKRGPETHYGQKAILFLPLPVSSDPARFLPLPGLPPALPEPPGILAPQPPDVGSSDPLSMVGPSQGRSPSYAS

SEQ ID NO: 15 - DNA, 591 bp, synthetic construct
  source 1..591: mol_type = other DNA
atgcaccaccaccaccaccacttcaacctgccccccggcaactacaagaagcccaagctgctgtactgcagcaacggcggccacttcctgaggatcctgcccgacggcaccgtggacggcaccagggacaggagcgacccccacatccagctgcagctgctggccgagagcgtgggcgaggtgtacatcaagagcaccgagaccggccagtacctggccatggacaccgacggcctgctgtacggcagccagacccccaacgaggagtgcctgttcctggagaggctggaggagaacagctacaacacctacatcagcaagaagcacgccgagaagaactggttcgtgggcctgaacaagaacggcagctgcaagaggggccccgagacccactacggccagaaggccatcctgttcctgcccctgcccgtgagcagcgaccccgccaggttcctgcccctgcccggcctgccccccgccctgcccgagccccccggcatcctggccccccagccccccgacgtgggcagcagcgaccccctgagcatggtgggccccagccagggcaggagccccagctacgccagctaa

SEQ ID NO: 16 - DNA, 591 bp, synthetic construct
  source 1..591: mol_type = other DNA
atgcaccaccaccaccaccacttcaacctgccccccggcaactacaagaagcccaagctgctgtactgcagcaacggcggccacttcctgaggatcctgcccgacggcaccgtggacggcaccagggacaggagcgacccccacatccagctgcagctgctggccgagagcgtgggcgaggtgtacatcaagagcaccgagaccggccagtacctggccatggacaccgacggcctgctgtacggcagccagacccccaacgaggagtgcctgttcctggagaggctggaggagaacagctacaacacctacatcagcaagaagcacgccgagaagaactggttcgtgggcctgaacaagaacggcagctgcaagaggggccccgagacccactacggccagaaggccatcctgttcctgcccctgcccgtgagcagcgaccccgccaggttcctgcccctgcccgccctgccccccgccctgcccgagccccccggcatcctggccccccagccccccgacgtgggcagcagcgaccccctgagcatggtgggccccagccagggcaggagccccagctacgccagctaa

SEQ ID NO: 17 - DNA, 591 bp, synthetic construct
  source 1..591: mol_type = other DNA
atgcaccaccaccaccaccacttcaacctgccccccggcaactacaagaagcccaagctgctgtactgcagcaacggcggccacttcctgaggatcctgcccgacggcaccgtggacggcaccagggacaggagcgacccccacatccagctgcagctgctggccgagagcgtgggcgaggtgtacatcaagagcaccgagaccggccagtacctggccatggacaccgacggcctgctgtacggcagccagacccccaacgaggagtgcctgttcctggagaggctggaggagaacagctacaacacctacatcagcaagaagcacgccgagaagaactggttcgtgggcctgaacaagaacggcagctgcaagaggggccccgagacccactacggccagaaggccatcctgttcctgcccctgcccgtgagcagcgaccccgccaggttcctgcccctgcccggcctgccccccgccctgcccgagccccccggcatcctggccccccagccccccgacgtgggcagcagcgaccccctgagcatggtgggccccagccaggagaggagccccagctacgccagctaa

SEQ ID NO: 18 - DNA, 649 bp, synthetic construct
  source 1..649: mol_type = other DNA
acatatggctagcatgactggtggacagcaaatgggtcgcggatccatgcaccaccaccaccaccacttcaacctgccccccgcaactacaagaagcccaagctgctgtactgcagcaacggcggccacttcctgaggatcctgcccgacggcaccgtggacggcaccagggaacaggagcgacccccacatccagctgcagctgctggccgagagcgtgggcgaggtgtacatcaagagcaccgagaccggccagtacctggccatggacaccgacggcctgctgtacggcagccagacccccaacgaggagtgcctgttcctggagaggctggaggagaacagctacaacacctacatcagcaagaagcacgccgagaagaactggttcgtgggcctgaacaagaacggcagctgcaagaggggccccgagacccactacggccagaaggccatcctgttcctgcccctgcccgtgagcagcgaccccgccaggttcctgcccctgcccggcctgccccccgccctgcccgagccccccggcatcctggccccccagccccccgacgtgggcagcagcgaccccctgagcatggtgggccccagccagggcaggagccccagctacgccagctaagattcgagctcc